SEQUENCE LISTING (70 sequences)

SEQ ID NO: 1 - RNA, 21 nt, synthetic construct
  misc_feature 1..21: note = Description of Artificial Sequence: Synthetic oligonucleotide
  source 1..21: mol_type = other RNA
cgaaacgtgaagaaataatga

SEQ ID NO: 2 - RNA, 23 nt, synthetic construct
  misc_feature 1..23: note = Description of Artificial Sequence: Synthetic oligonucleotide
  source 1..23: mol_type = other RNA
tcattatttcttcacgtttcggc

SEQ ID NO: 3 - RNA, 21 nt, synthetic construct
  misc_feature 1..21: note = Description of Artificial Sequence: Synthetic oligonucleotide
  source 1..21: mol_type = other RNA
cctaaacgcatgtcttcagaa

SEQ ID NO: 4 - RNA, 23 nt, synthetic construct
  misc_feature 1..23: note = Description of Artificial Sequence: Synthetic oligonucleotide
  source 1..23: mol_type = other RNA
ttctgaagacatgcgtttaggca

SEQ ID NO: 5 - RNA, 21 nt, synthetic construct
  misc_feature 1..21: note = Description of Artificial Sequence: Synthetic oligonucleotide
  source 1..21: mol_type = other RNA
gagatgcttttactgactcta

SEQ ID NO: 6 - RNA, 23 nt, synthetic construct
  misc_feature 1..23: note = Description of Artificial Sequence: Synthetic oligonucleotide
  source 1..23: mol_type = other RNA
tagagtcagtaaaagcatctctt

SEQ ID NO: 7 - RNA, 21 nt, synthetic construct
  misc_feature 1..21: note = Description of Artificial Sequence: Synthetic oligonucleotide
  source 1..21: mol_type = other RNA
aaacgtgaagaaataatggaa

SEQ ID NO: 8 - RNA, 23 nt, synthetic construct
  misc_feature 1..23: note = Description of Artificial Sequence: Synthetic oligonucleotide
  source 1..23: mol_type = other RNA
ttccattatttcttcacgtttcg

SEQ ID NO: 9 - RNA, 21 nt, synthetic construct
  misc_feature 1..21: note = Description of Artificial Sequence: Synthetic oligonucleotide
  source 1..21: mol_type = other RNA
gctcctgtctctactatgcca

SEQ ID NO: 10 - RNA, 23 nt, synthetic construct
  misc_feature 1..23: note = Description of Artificial Sequence: Synthetic oligonucleotide
  source 1..23: mol_type = other RNA
tggcatagtagagacaggagctg

SEQ ID NO: 11 - RNA, 21 nt, synthetic construct
  misc_feature 1..21: note = Description of Artificial Sequence: Synthetic oligonucleotide
  source 1..21: mol_type = other RNA
tggttctccagctcctgtcta

SEQ ID NO: 12 - RNA, 23 nt, synthetic construct
  misc_feature 1..23: note = Description of Artificial Sequence: Synthetic oligonucleotide
  source 1..23: mol_type = other RNA
tagacaggagctggagaaccatg

SEQ ID NO: 13 - RNA, 21 nt, synthetic construct
  misc_feature 1..21: note = Description of Artificial Sequence: Synthetic oligonucleotide
  source 1..21: mol_type = other RNA
gcctaaacgcatgtcttcaga

SEQ ID NO: 14 - RNA, 23 nt, synthetic construct
  misc_feature 1..23: note = Description of Artificial Sequence: Synthetic oligonucleotide
  source 1..23: mol_type = other RNA
tctgaagacatgcgtttaggcat

SEQ ID NO: 15 - RNA, 21 nt, synthetic construct
  misc_feature 1..21: note = Description of Artificial Sequence: Synthetic oligonucleotide
  source 1..21: mol_type = other RNA
ttatcagtctcagcatcctca

SEQ ID NO: 16 - RNA, 23 nt, synthetic construct
  misc_feature 1..23: note = Description of Artificial Sequence: Synthetic oligonucleotide
  source 1..23: mol_type = other RNA
tgaggatgctgagactgataatg

SEQ ID NO: 17 - RNA, 21 nt, synthetic construct
  misc_feature 1..21: note = Description of Artificial Sequence: Synthetic oligonucleotide
  source 1..21: mol_type = other RNA
tctccagctcctgtctctaca

SEQ ID NO: 18 - RNA, 23 nt, synthetic construct
  misc_feature 1..23: note = Description of Artificial Sequence: Synthetic oligonucleotide
  source 1..23: mol_type = other RNA
tgtagagacaggagctggagaac

SEQ ID NO: 19 - RNA, 21 nt, synthetic construct
  misc_feature 1..21: note = Description of Artificial Sequence: Synthetic oligonucleotide
  source 1..21: mol_type = other RNA
gctcatggttctccagctcca

SEQ ID NO: 20 - RNA, 23 nt, synthetic construct
  misc_feature 1..23: note = Description of Artificial Sequence: Synthetic oligonucleotide
  source 1..23: mol_type = other RNA
tggagctggagaaccatgagcag

SEQ ID NO: 21 - RNA, 21 nt, synthetic construct
  misc_feature 1..21: note = Description of Artificial Sequence: Synthetic oligonucleotide
  source 1..21: mol_type = other RNA
cagtcaggtcccaactctcta

SEQ ID NO: 22 - RNA, 23 nt, synthetic construct
  misc_feature 1..23: note = Description of Artificial Sequence: Synthetic oligonucleotide
  source 1..23: mol_type = other RNA
tagagagttgggacctgactggt

SEQ ID NO: 23 - RNA, 21 nt, synthetic construct
  misc_feature 1..21: note = Description of Artificial Sequence: Synthetic oligonucleotide
  source 1..21: mol_type = other RNA
gagtagttaatggaggtgtta

SEQ ID NO: 24 - RNA, 23 nt, synthetic construct
  misc_feature 1..23: note = Description of Artificial Sequence: Synthetic oligonucleotide
  source 1..23: mol_type = other RNA
taacacctccattaactactctt

SEQ ID NO: 25 - RNA, 21 nt, synthetic construct
  misc_feature 1..21: note = Description of Artificial Sequence: Synthetic oligonucleotide
  source 1..21: mol_type = other RNA
agtcaggtcccaactctctta

SEQ ID NO: 26 - RNA, 23 nt, synthetic construct
  misc_feature 1..23: note = Description of Artificial Sequence: Synthetic oligonucleotide
  source 1..23: mol_type = other RNA
taagagagttgggacctgactgg

SEQ ID NO: 27 - RNA, 21 nt, synthetic construct
  misc_feature 1..21: note = Description of Artificial Sequence: Synthetic oligonucleotide
  source 1..21: mol_type = other RNA
cccaccttctcgctaccagta

SEQ ID NO: 28 - RNA, 23 nt, synthetic construct
  misc_feature 1..23: note = Description of Artificial Sequence: Synthetic oligonucleotide
  source 1..23: mol_type = other RNA
tactggtagcgagaaggtgggcg

SEQ ID NO: 29 - RNA, 21 nt, synthetic construct
  misc_feature 1..21: note = Description of Artificial Sequence: Synthetic oligonucleotide
  source 1..21: mol_type = other RNA
caagacatatagagcagtaca

SEQ ID NO: 30 - RNA, 23 nt, synthetic construct
  misc_feature 1..23: note = Description of Artificial Sequence: Synthetic oligonucleotide
  source 1..23: mol_type = other RNA
tgtactgctctatatgtcttggc

SEQ ID NO: 31 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methylguanosine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = OTHER; note = 2'-fluoroguanosine
  modified_base 8: mod_base = um
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = gm
  modified_base 13: mod_base = OTHER; note = 2'-O-methyladenosine
  modified_base 14: mod_base = OTHER; note = 2'-O-methyladenosine
  modified_base 15: mod_base = OTHER; note = 2'-O-methyladenosine
  modified_base 16: mod_base = um
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyluridine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
  source 1..21: mol_type = other RNA
cgaaacgtgaagaaataatga

SEQ ID NO: 32 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-phosphate substitution
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = um
  modified_base 5: mod_base = um
  modified_base 6: mod_base = OTHER; note = 2'-fluoroadenosine
  modified_base 7: mod_base = um
  modified_base 8: mod_base = um
  modified_base 9: mod_base = um
  modified_base 10: mod_base = cm
  modified_base 11: mod_base = um
  modified_base 12: mod_base = um
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluoroguanosine
  modified_base 17: mod_base = um
  modified_base 18: mod_base = um
  modified_base 19: mod_base = um
  modified_base 20: mod_base = cm
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = cm
  source 1..23: mol_type = other RNA
tcattatttcttcacgtttcggc

SEQ ID NO: 33 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = um
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluorocytidine
  modified_base 8: mod_base = gm
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluorouridine
  modified_base 12: mod_base = gm
  modified_base 13: mod_base = um
  modified_base 14: mod_base = cm
  modified_base 15: mod_base = um
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
cctaaacgcatgtcttcagaa

SEQ ID NO: 34 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = cm
  modified_base 4: mod_base = um
  modified_base 5: mod_base = gm
  modified_base 6: mod_base = OTHER; note = 2'-fluoroadenosine
  modified_base 7: mod_base = OTHER; note = 2'-O-methyladenosine
  modified_base 8: mod_base = gm
  modified_base 9: mod_base = OTHER; note = 2'-O-methyladenosine
  modified_base 10: mod_base = cm
  modified_base 11: mod_base = OTHER; note = 2'-O-methyladenosine
  modified_base 12: mod_base = um
  modified_base 13: mod_base = gm
  modified_base 14: mod_base = OTHER; note = 2'-fluorocytidine
  modified_base 15: mod_base = gm
  modified_base 16: mod_base = OTHER; note = 2'-fluorouridine
  modified_base 17: mod_base = um
  modified_base 18: mod_base = um
  modified_base 19: mod_base = OTHER; note = 2'-O-methyladenosine
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methylcytidine with a 3'-phosphorothioate linkage
  modified_base 23: mod_base = OTHER; note = 2'-O-methyladenosine
  source 1..23: mol_type = other RNA
ttctgaagacatgcgtttaggca

SEQ ID NO: 35 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = OTHER; note = 2'-O-methyladenosine
  modified_base 5: mod_base = um
  modified_base 6: mod_base = gm
  modified_base 7: mod_base = OTHER; note = 2'-fluorocytidine
  modified_base 8: mod_base = um
  modified_base 9: mod_base = OTHER; note = 2'-fluorouridine
  modified_base 10: mod_base = OTHER; note = 2'-fluorouridine
  modified_base 11: mod_base = OTHER; note = 2'-fluorouridine
  modified_base 12: mod_base = OTHER; note = 2'-O-methyladenosine
  modified_base 13: mod_base = cm
  modified_base 14: mod_base = um
  modified_base 15: mod_base = gm
  modified_base 16: mod_base = OTHER; note = 2'-O-methyladenosine
  modified_base 17: mod_base = cm
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
  source 1..21: mol_type = other RNA
gagatgcttttactgactcta

SEQ ID NO: 36 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroadenosine with a 3'-phosphorothioate linkage
  modified_base 3: mod_base = gm
  modified_base 4: mod_base = OTHER; note = 2'-O-methyladenosine
  modified_base 5: mod_base = gm
  modified_base 6: mod_base = OTHER; note = 2'-fluorouridine
  modified_base 7: mod_base = cm
  modified_base 8: mod_base = OTHER; note = 2'-O-methyladenosine
  modified_base 9: mod_base = gm
  modified_base 10: mod_base = um
  modified_base 11: mod_base = OTHER; note = 2'-O-methyladenosine
  modified_base 12: mod_base = OTHER; note = 2'-O-methyladenosine
  modified_base 13: mod_base = OTHER; note = 2'-O-methyladenosine
  modified_base 14: mod_base = OTHER; note = 2'-fluoroadenosine
  modified_base 15: mod_base = gm
  modified_base 16: mod_base = OTHER; note = 2'-fluorocytidine
  modified_base 17: mod_base = OTHER; note = 2'-O-methyladenosine
  modified_base 18: mod_base = um
  modified_base 19: mod_base = cm
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = um
  source 1..23: mol_type = other RNA
tagagtcagtaaaagcatctctt

SEQ ID NO: 37 - RNA, 21 nt, synthetic construct
  misc_feature 1..21: note = Description of Artificial Sequence: Synthetic oligonucleotide
  modified_base 1: mod_base = OTHER; note = 2'-O-methyladenosine with a 3'-phosphorothioate linkage
  modified_base 2: mod_base = OTHER; note = 2'-O-methyladenosine with a 3'-phosphorothioate linkage
  modified_base 3: mod_base = OTHER; note = 2'-O-methyladenosine
  modified_base 4: mod_base = cm
  modified_base 5: mod_base = gm
  modified_base 6: mod_base = um
  modified_base 7: mod_base = OTHER; note = 2'-fluoroguanosine
  modified_base 8: mod_base = OTHER; note = 2'-O-methyladenosine
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluoroadenosine
  modified_base 12: mod_base = OTHER; note = 2'-O-methyladenosine
  modified_base 13: mod_base = OTHER; note = 2'-O-methyladenosine
  modified_base 14: mod_base = um
  modified_base 15: mod_base = OTHER; note = 2'-O-methyladenosine
  modified_base 16: mod_base = OTHER; note = 2'-O-methyladenosine
  modified_base 17: mod_base = um
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
aaacgtgaagaaataatggaa

SEQ ID NO: 38 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = cm
  modified_base 4: mod_base = cm
  modified_base 5: mod_base = OTHER; note = 2'-O-methyladenosine
  modified_base 6: mod_base = OTHER; note = 2'-fluorouridine
  modified_base 7: mod_base = um
  modified_base 8: mod_base = OTHER; note = 2'-O-methyladenosine
  modified_base 9: mod_base = um
  modified_base 10: mod_base = um
  modified_base 11: mod_base = um
  modified_base 12: mod_base = cm
  modified_base 13: mod_base = um
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = cm
  modified_base 18: mod_base = gm
  modified_base 19: mod_base = um
  modified_base 20: mod_base = um
  modified_base 21: mod_base = OTHER; note = 2'-O-methyluridine with a 3'-phosphorothioate linkage
  modified_base 22: mod_base = OTHER; note = 2'-O-methylcytidine with a 3'-phosphorothioate linkage
  modified_base 23: mod_base = gm
  source 1..23: mol_type = other RNA
ttccattatttcttcacgtttcg

SEQ ID NO: 39 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methylcytidine with a 3'-phosphorothioate linkage
  modified_base 3: mod_base = um
  modified_base 4: mod_base = cm
  modified_base 5: mod_base = cm
  modified_base 6: mod_base = um
  modified_base 7: mod_base = OTHER; note = 2'-fluoroguanosine
  modified_base 8: mod_base = um
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = um
  modified_base 16: mod_base = OTHER; note = 2'-O-methyladenosine
  modified_base 17: mod_base = um
  modified_base 18: mod_base = gm
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
gctcctgtctctactatgcca

SEQ ID NO: 40 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-fluorouridine
  modified_base 7: mod_base = OTHER; note = 2'-O-methyladenosine
  modified_base 8: mod_base = gm
  modified_base 9: mod_base = um
  modified_base 10: mod_base = OTHER; note = 2'-O-methyladenosine
  modified_base 11: mod_base = gm
  modified_base 12: mod_base = OTHER; note = 2'-O-methyladenosine
  modified_base 13: mod_base = gm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = gm
  modified_base 18: mod_base = gm
  modified_base 19: mod_base = OTHER; note = 2'-O-methyladenosine
  modified_base 20: mod_base = gm
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = gm
  source 1..23: mol_type = other RNA
tggcatagtagagacaggagctg

SEQ ID NO: 41 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = gm
  modified_base 4: mod_base = um
  modified_base 5: mod_base = um
  modified_base 6: mod_base = cm
  modified_base 7: mod_base = OTHER; note = 2'-fluorouridine
  modified_base 8: mod_base = cm
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = cm
  modified_base 16: mod_base = um
  modified_base 17: mod_base = gm
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
  source 1..21: mol_type = other RNA
tggttctccagctcctgtcta

SEQ ID NO: 42 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroadenosine with a 3'-phosphorothioate linkage
  modified_base 3: mod_base = gm
  modified_base 4: mod_base = OTHER; note = 2'-O-methyladenosine
  modified_base 5: mod_base = cm
  modified_base 6: mod_base = OTHER; note = 2'-fluoroadenosine
  modified_base 7: mod_base = gm
  modified_base 8: mod_base = gm
  modified_base 9: mod_base = OTHER; note = 2'-O-methyladenosine
  modified_base 10: mod_base = gm
  modified_base 11: mod_base = cm
  modified_base 12: mod_base = um
  modified_base 13: mod_base = gm
  modified_base 14: mod_base = OTHER; note = 2'-fluoroguanosine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = OTHER; note = 2'-O-methyladenosine
  modified_base 19: mod_base = cm
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = gm
  source 1..23: mol_type = other RNA
tagacaggagctggagaaccatg

SEQ ID NO: 43 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methylcytidine with a 3'-phosphorothioate linkage
  modified_base 3: mod_base = cm
  modified_base 4: mod_base = um
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroadenosine
  modified_base 8: mod_base = cm
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluoroadenosine
  modified_base 12: mod_base = um
  modified_base 13: mod_base = gm
  modified_base 14: mod_base = um
  modified_base 15: mod_base = cm
  modified_base 16: mod_base = um
  modified_base 17: mod_base = um
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
  source 1..21: mol_type = other RNA
gcctaaacgcatgtcttcaga

SEQ ID NO: 44 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-phosphate substitution
  modified_base 2: mod_base = OTHER; note = 2'-fluorocytidine with a 3'-phosphorothioate linkage
  modified_base 3: mod_base = um
  modified_base 4: mod_base = gm
  modified_base 5: mod_base = OTHER; note = 2'-O-methyladenosine
  modified_base 6: mod_base = OTHER; note = 2'-fluoroadenosine
  modified_base 7: mod_base = gm
  modified_base 8: mod_base = OTHER; note = 2'-O-methyladenosine
  modified_base 9: mod_base = cm
  modified_base 10: mod_base = OTHER; note = 2'-O-methyladenosine
  modified_base 11: mod_base = um
  modified_base 12: mod_base = gm
  modified_base 13: mod_base = cm
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = um
  modified_base 18: mod_base = OTHER; note = 2'-O-methyladenosine
  modified_base 19: mod_base = gm
  modified_base 20: mod_base = gm
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = um
  source 1..23: mol_type = other RNA
tctgaagacatgcgtttaggcat

SEQ ID NO: 45 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methyluridine with a 3'-phosphorothioate linkage
  modified_base 3: mod_base = OTHER; note = 2'-O-methyladenosine
  modified_base 4: mod_base = um
  modified_base 5: mod_base = cm
  modified_base 6: mod_base = OTHER; note = 2'-O-methyladenosine
  modified_base 7: mod_base = OTHER; note = 2'-fluoroguanosine
  modified_base 8: mod_base = um
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = OTHER; note = 2'-O-methyladenosine
  modified_base 13: mod_base = gm
  modified_base 14: mod_base = cm
  modified_base 15: mod_base = OTHER; note = 2'-O-methyladenosine
  modified_base 16: mod_base = um
  modified_base 17: mod_base = cm
  modified_base 18: mod_base = cm
  modified_base 19: mod_base = OTHER; note = 2'-O-methyluridine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ttatcagtctcagcatcctca

SEQ ID NO: 46 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = OTHER; note = 2'-O-methyladenosine
  modified_base 4: mod_base = gm
  modified_base 5: mod_base = gm
  modified_base 6: mod_base = OTHER; note = 2'-fluoroadenosine
  modified_base 7: mod_base = um
  modified_base 8: mod_base = gm
  modified_base 9: mod_base = cm
  modified_base 10: mod_base = um
  modified_base 11: mod_base = gm
  modified_base 12: mod_base = OTHER; note = 2'-O-methyladenosine
  modified_base 13: mod_base = gm
  modified_base 14: mod_base = OTHER; note = 2'-fluoroadenosine
  modified_base 15: mod_base = cm
  modified_base 16: mod_base = OTHER; note = 2'-fluorouridine
  modified_base 17: mod_base = gm
  modified_base 18: mod_base = OTHER; note = 2'-O-methyladenosine
  modified_base 19: mod_base = um
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = gm
  source 1..23: mol_type = other RNA
tgaggatgctgagactgataatg

SEQ ID NO: 47 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylcytidine with a 3'-phosphorothioate linkage
  modified_base 3: mod_base = um
  modified_base 4: mod_base = cm
  modified_base 5: mod_base = cm
  modified_base 6: mod_base = OTHER; note = 2'-O-methyladenosine
  modified_base 7: mod_base = OTHER; note = 2'-fluoroguanosine
  modified_base 8: mod_base = cm
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = gm
  modified_base 14: mod_base = um
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
tctccagctcctgtctctaca

SEQ ID NO: 48 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = um
  modified_base 4: mod_base = OTHER; note = 2'-O-methyladenosine
  modified_base 5: mod_base = gm
  modified_base 6: mod_base = OTHER; note = 2'-fluoroadenosine
  modified_base 7: mod_base = gm
  modified_base 8: mod_base = OTHER; note = 2'-O-methyladenosine
  modified_base 9: mod_base = cm
  modified_base 10: mod_base = OTHER; note = 2'-O-methyladenosine
  modified_base 11: mod_base = gm
  modified_base 12: mod_base = gm
  modified_base 13: mod_base = OTHER; note = 2'-O-methyladenosine
  modified_base 14: mod_base = OTHER; note = 2'-fluoroguanosine
  modified_base 15: mod_base = cm
  modified_base 16: mod_base = OTHER; note = 2'-fluorouridine
  modified_base 17: mod_base = gm
  modified_base 18: mod_base = gm
  modified_base 19: mod_base = OTHER; note = 2'-O-methyladenosine
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = cm
  source 1..23: mol_type = other RNA
tgtagagacaggagctggagaac

SEQ ID NO: 49 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methylcytidine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = um
  modified_base 7: mod_base = OTHER; note = 2'-fluoroguanosine
  modified_base 8: mod_base = gm
  modified_base 9: mod_base = OTHER; note = 2'-fluorouridine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = cm
  modified_base 14: mod_base = cm
  modified_base 15: mod_base = OTHER; note = 2'-O-methyladenosine
  modified_base 16: mod_base = gm
  modified_base 17: mod_base = cm
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
gctcatggttctccagctcca

SEQ ID NO: 50 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = gm
  modified_base 4: mod_base = OTHER; note = 2'-O-methyladenosine
  modified_base 5: mod_base = gm
  modified_base 6: mod_base = OTHER; note = 2'-fluorocytidine
  modified_base 7: mod_base = um
  modified_base 8: mod_base = gm
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-O-methyladenosine
  modified_base 13: mod_base = OTHER; note = 2'-O-methyladenosine
  modified_base 14: mod_base = OTHER; note = 2'-fluorocytidine
  modified_base 15: mod_base = cm
  modified_base 16: mod_base = OTHER; note = 2'-fluoroadenosine
  modified_base 17: mod_base = um
  modified_base 18: mod_base = gm
  modified_base 19: mod_base = OTHER; note = 2'-O-methyladenosine
  modified_base 20: mod_base = gm
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = gm
  source 1..23: mol_type = other RNA
tggagctggagaaccatgagcag

SEQ ID NO: 51 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = um
  modified_base 5: mod_base = cm
  modified_base 6: mod_base = OTHER; note = 2'-O-methyladenosine
  modified_base 7: mod_base = OTHER; note = 2'-fluoroguanosine
  modified_base 8: mod_base = gm
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = cm
  modified_base 13: mod_base = OTHER; note = 2'-O-methyladenosine
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
  source 1..21: mol_type = other RNA
cagtcaggtcccaactctcta

SEQ ID NO: 52 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroadenosine with a 3'-phosphorothioate linkage
  modified_base 3: mod_base = gm
  modified_base 4: mod_base = OTHER; note = 2'-O-methyladenosine
  modified_base 5: mod_base = gm
  modified_base 6: mod_base = OTHER; note = 2'-fluoroadenosine
  modified_base 7: mod_base = gm
  modified_base 8: mod_base = um
  modified_base 9: mod_base = um
  modified_base 10: mod_base = gm
  modified_base 11: mod_base = gm
  modified_base 12: mod_base = gm
  modified_base 13: mod_base = OTHER; note = 2'-O-methyladenosine
  modified_base 14: mod_base = OTHER; note = 2'-fluorocytidine
  modified_base 15: mod_base = cm
  modified_base 16: mod_base = OTHER; note = 2'-fluorouridine
  modified_base 17: mod_base = gm
  modified_base 18: mod_base = OTHER; note = 2'-O-methyladenosine
  modified_base 19: mod_base = cm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = um
  source 1..23: mol_type = other RNA
tagagagttgggacctgactggt

SEQ ID NO: 53 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = gm
  modified_base 7: mod_base = OTHER; note = 2'-fluorouridine
  modified_base 8: mod_base = um
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluorouridine
  modified_base 12: mod_base = gm
  modified_base 13: mod_base = gm
  modified_base 14: mod_base = OTHER; note = 2'-O-methyladenosine
  modified_base 15: mod_base = gm
  modified_base 16: mod_base = gm
  modified_base 17: mod_base = um
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
  source 1..21: mol_type = other RNA
gagtagttaatggaggtgtta

SEQ ID NO: 54 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroadenosine with a 3'-phosphorothioate linkage
  modified_base 3: mod_base = OTHER; note = 2'-O-methyladenosine
  modified_base 4: mod_base = cm
  modified_base 5: mod_base = OTHER; note = 2'-O-methyladenosine
  modified_base 6: mod_base = OTHER; note = 2'-fluorocytidine
  modified_base 7: mod_base = cm
  modified_base 8: mod_base = um
  modified_base 9: mod_base = cm
  modified_base 10: mod_base = cm
  modified_base 11: mod_base = OTHER; note = 2'-O-methyladenosine
  modified_base 12: mod_base = um
  modified_base 13: mod_base = um
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluorocytidine
  modified_base 17: mod_base = um
  modified_base 18: mod_base = OTHER; note = 2'-O-methyladenosine
  modified_base 19: mod_base = cm
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = um
  source 1..23: mol_type = other RNA
taacacctccattaactactctt

SEQ ID NO: 55 - RNA, 21 nt, synthetic construct
  misc_feature 1..21: note = Description of Artificial Sequence: Synthetic oligonucleotide
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = gm
  modified_base 7: mod_base = OTHER; note = 2'-fluoroguanosine
  modified_base 8: mod_base = um
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-O-methyladenosine
  modified_base 13: mod_base = OTHER; note = 2'-O-methyladenosine
  modified_base 14: mod_base = cm
  modified_base 15: mod_base = um
  modified_base 16: mod_base = cm
  modified_base 17: mod_base = um
  modified_base 18: mod_base = cm
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
  source 1..21: mol_type = other RNA
agtcaggtcccaactctctta

SEQ ID NO: 56 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroadenosine with a 3'-phosphorothioate linkage
  modified_base 3: mod_base = OTHER; note = 2'-O-methyladenosine
  modified_base 4: mod_base = gm
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methyladenosine
  modified_base 8: mod_base = gm
  modified_base 9: mod_base = um
  modified_base 10: mod_base = um
  modified_base 11: mod_base = gm
  modified_base 12: mod_base = gm
  modified_base 13: mod_base = gm
  modified_base 14: mod_base = OTHER; note = 2'-fluoroadenosine
  modified_base 15: mod_base = cm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = gm
  source 1..23: mol_type = other RNA
taagagagttgggacctgactgg

SEQ ID NO: 57 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = cm
  modified_base 4: mod_base = OTHER; note = 2'-O-methyladenosine
  modified_base 5: mod_base = cm
  modified_base 6: mod_base = cm
  modified_base 7: mod_base = OTHER; note = 2'-fluorouridine
  modified_base 8: mod_base = um
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = gm
  modified_base 13: mod_base = cm
  modified_base 14: mod_base = um
  modified_base 15: mod_base = OTHER; note = 2'-O-methyladenosine
  modified_base 16: mod_base = cm
  modified_base 17: mod_base = cm
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
  source 1..21: mol_type = other RNA
cccaccttctcgctaccagta

SEQ ID NO: 58 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroadenosine with a 3'-phosphorothioate linkage
  modified_base 3: mod_base = cm
  modified_base 4: mod_base = um
  modified_base 5: mod_base = gm
  modified_base 6: mod_base = OTHER; note = 2'-fluoroguanosine
  modified_base 7: mod_base = um
  modified_base 8: mod_base = OTHER; note = 2'-O-methyladenosine
  modified_base 9: mod_base = gm
  modified_base 10: mod_base = cm
  modified_base 11: mod_base = gm
  modified_base 12: mod_base = OTHER; note = 2'-O-methyladenosine
  modified_base 13: mod_base = gm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = gm
  modified_base 18: mod_base = um
  modified_base 19: mod_base = gm
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methylcytidine with a 3'-phosphorothioate linkage
  modified_base 23: mod_base = gm
  source 1..23: mol_type = other RNA
tactggtagcgagaaggtgggcg

SEQ ID NO: 59 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = OTHER; note = 2'-O-methyladenosine
  modified_base 4: mod_base = gm
  modified_base 5: mod_base = OTHER; note = 2'-O-methyladenosine
  modified_base 6: mod_base = cm
  modified_base 7: mod_base = OTHER; note = 2'-fluoroadenosine
  modified_base 8: mod_base = um
  modified_base 9: mod_base = OTHER; note = 2'-fluoroadenosine
  modified_base 10: mod_base = OTHER; note = 2'-fluorouridine
  modified_base 11: mod_base = OTHER; note = 2'-fluoroadenosine
  modified_base 12: mod_base = gm
  modified_base 13: mod_base = OTHER; note = 2'-O-methyladenosine
  modified_base 14: mod_base = gm
  modified_base 15: mod_base = cm
  modified_base 16: mod_base = OTHER; note = 2'-O-methyladenosine
  modified_base 17: mod_base = gm
  modified_base 18: mod_base = um
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
caagacatatagagcagtaca

SEQ ID NO: 60 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = um
  modified_base 4: mod_base = OTHER; note = 2'-O-methyladenosine
  modified_base 5: mod_base = cm
  modified_base 6: mod_base = OTHER; note = 2'-fluorouridine
  modified_base 7: mod_base = gm
  modified_base 8: mod_base = cm
  modified_base 9: mod_base = um
  modified_base 10: mod_base = cm
  modified_base 11: mod_base = um
  modified_base 12: mod_base = OTHER; note = 2'-O-methyladenosine
  modified_base 13: mod_base = um
  modified_base 14: mod_base = OTHER; note = 2'-fluoroadenosine
  modified_base 15: mod_base = um
  modified_base 16: mod_base = OTHER; note = 2'-fluoroguanosine
  modified_base 17: mod_base = um
  modified_base 18: mod_base = cm
  modified_base 19: mod_base = um
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = cm
  source 1..23: mol_type = other RNA
tgtactgctctatatgtcttggc

SEQ ID NO: 61 - DNA, 4341 bp, Homo sapiens
  source 1..4341: mol_type = genomic DNA
agagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactga

SEQ ID NO: 62 - protein, 1153 aa, Homo sapiens
MSLKPQQQQQQQQQQQQQQQQQQQQQQQPPPAAANVRKPGGSGLLASPAAAPSPSSSSVSSSSATAPSSVVAATSGGGRPGLGRGRNSNKGLPQSTISFDGIYANMRMVHILTSVVGSKCEVQVKNGGIYEGVFKTYSPKCDLVLDAAHEKSTESSSGPKREEIMESILFKCSDFVVVQFKDMDSSYAKRDAFTDSAISAKVNGEHKEKDLEPWDAGELTANEELEALENDVSNGWDPNDMFRYNEENYGVVSTYDSSLSSYTVPLERDNSEEFLKREARANQLAEEIESSAQYKARVALENDDRSEEEKYTAVQRNSSEREGHSINTRENKYIPPGQRNREVISWGSGRQNSPRMGQPGSGSMPSRSTSHTSDFNPNSGSDQRVVNGGVPWPSPCPSPSSRPPSRYQSGPNSLPPRAATPTRPPSRPPSRPSRPPSHPSAHGSPAPVSTMPKRMSSEGPPRMSPKAQRHPRNHRVSAGRGSISSGLEFVSHNPPSEAATPPVARTSPSGGTWSSVVSGVPRLSPKTHRPRSPRQNSIGNTPSGPVLASPQAGIIPTEAVAMPIPAASPTPASPASNRAVTPSSEAKDSRLQDQRQNSPAGNKENIKPNETSPSFSKAENKGISPVVSEHRKQIDDLKKFKNDFRLQPSSTSESMDQLLNKNREGEKSRDLIKDKIEPSAKDSFIENSSSNCTSGSSKPNSPSISPSILSNTEHKRGPEVTSQGVQTSSPACKQEKDDKEEKKDAAEQVRKSTLNPNAKEFNPRSFSQPKPSTTPTSPRPQAQPSPSMVGHQQPTPVYTQPVCFAPNMMYPVPVSPGVQPLYPIPMTPMPVNQAKTYRAVPNMPQQRQDQHHQSAMMHPASAAGPPIAATPPAYSTQYVAYSPQQFPNQPLVQHVPHYQSQHPHVYSPVIQGNARMMAPPTHAQPGLVSSSATQYGAHEQTHAMYACPKLPYNKETSPSFYFAISTGSLAQQYAHPNATLHPHTPHPQPSATPTGQQQSQHGGSHPAPSPVQHHQHQAAQALHLASPQQQSAIYHAGLAPTPPSMTPASNTQSPQNSFPAAQQTVFTIHPSHVQPAYTNPPHMAHVPQAHVQSGMVPSHPTAHAPMMLMTTQPPGGPQAALAQSALQPIPVSTTAHFPYMTHPSVQAHHQQQL

SEQ ID NO: 63 - DNA, 3993 bp, Homo sapiens
  source 1..3993: mol_type = genomic DNA
cccgagaaagcaacccagcgcgccgcccgctcctcacgtgtccctcccggccccggggccacctcacgttctgcttccgtctgacccctccgacttccgaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcagtgcgccagtgaggctctggcaaggtgtgggctagagatgcgactcagttggatctatctctcagaaggctaccttgctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactgaaaaaaaaaaa

SEQ ID NO: 64 - protein, 1073 aa, Homo sapiens
MRMVHILTSVVGSKCEVQVKNGGIYEGVFKTYSPKCDLVLDAAHEKSTESSSGPKREEIMESILFKCSDFVVVQFKDMDSSYAKRDAFTDSAISAKVNGEHKEKDLEPWDAGELTANEELEALENDVSNGWDPNDMFRYNEENYGVVSTYDSSLSSYTVPLERDNSEEFLKREARANQLAEEIESSAQYKARVALENDDRSEEEKYTAVQRNSSEREGHSINTRENKYIPPGQRNREVISWGSGRQNSPRMGQPGSGSMPSRSTSHTSDFNPNSGSDQRVVNGGVPWPSPCPSPSSRPPSRYQSGPNSLPPRAATPTRPPSRPPSRPSRPPSHPSAHGSPAPVSTMPKRMSSEGPPRMSPKAQRHPRNHRVSAGRGSISSGLEFVSHNPPSEAATPPVARTSPSGGTWSSVVSGVPRLSPKTHRPRSPRQNSIGNTPSGPVLASPQAGIIPTEAVAMPIPAASPTPASPASNRAVTPSSEAKDSRLQDQRQNSPAGNKENIKPNETSPSFSKAENKGISPVVSEHRKQIDDLKKFKNDFRLQPSSTSESMDQLLNKNREGEKSRDLIKDKIEPSAKDSFIENSSSNCTSGSSKPNSPSISPSILSNTEHKRGPEVTSQGVQTSSPACKQEKDDKEEKKDAAEQVRKSTLNPNAKEFNPRSFSQPKPSTTPTSPRPQAQPSPSMVGHQQPTPVYTQPVCFAPNMMYPVPVSPGVQPLYPIPMTPMPVNQAKTYRAVPNMPQQRQDQHHQSAMMHPASAAGPPIAATPPAYSTQYVAYSPQQFPNQPLVQHVPHYQSQHPHVYSPVIQGNARMMAPPTHAQPGLVSSSATQYGAHEQTHAMYACPKLPYNKETSPSFYFAISTGSLAQQYAHPNATLHPHTPHPQPSATPTGQQQSQHGGSHPAPSPVQHHQHQAAQALHLASPQQQSAIYHAGLAPTPPSMTPASNTQSPQNSFPAAQQTVFTIHPSHVQPAYTNPPHMAHVPQCASEALARCGLEMRLSWIYLSEGYLAHVQSGMVPSHPTAHAPMMLMTTQPPGGPQAALAQSALQPIPVSTTAHFPYMTHPSVQAHHQQQL

SEQ ID NO: 65 - DNA, 3755 bp, Homo sapiens
  source 1..3755: mol_type = genomic DNA
cccgagaaagcaacccagcgcgccgcccgctcctcacgtgtccctcccggccccggggccacctcacgttctgcttccgtctgacccctccgacttccgatttcttttgatggaatctatgcaaatatgaggatggttcatatacttacatcagttgtttgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgtttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactgaaaaaaaaaaa

SEQ ID NO: 66 - protein, 1006 aa, Homo sapiens
MRMVHILTSVVCDLVLDAAHEKSTESSSGPKREEIMESILFKCSDFVVVQFKDMDSSYAKRDAFTDSAISAKVNGEHKEKDLEPWDAGELTANEELEALENDVSNGWDPNDMFRYNEENYGVVSTYDSSLSSYTVPLERDNSEEFLKREARANQLAEEIESSAQYKARVALENDDRSEEEKYTAVQRNSSEREGHSINTRENKYIPPGQRNREVISWGSGRQNSPRMGQPGSGSMPSRSTSHTSDFNPNSGSDQRVVNGGVPWPSPCPSPSSRPPSRYQSGPNSLPPRAATPTRPPSRPPSRPSRPPSHPSAHGSPAPVSTMPKRMSSEGPPRMSPKAQRHPRNHRVSAGRGSISSGLEFVSHNPPSEAATPPVARTSPSGGTWSSVVSGVPRLSPKTHRPRSPRQNSIGNTPSGPVLASPQAGIIPTEAVAMPIPAASPTPASPASNRAVTPSSEAKDSRLQDQRQNSPAGNKENIKPNETSPSFSKAENKGISPVVSEHRKQIDDLKKFKNDFRLQPSSTSESMDQLLNKNREGEKSRDLIKDKIEPSAKDSFIENSSSNCTSGSSKPNSPSISPSILSNTEHKRGPEVTSQGVQTSSPACKQEKDDKEEKKDAAEQVRKSTLNPNAKEFNPRSFSQPKPSTTPTSPRPQAQPSPSMVGHQQPTPVYTQPVCFAPNMMYPVPVSPGVQPLYPIPMTPMPVNQAKTYRAVPNMPQQRQDQHHQSAMMHPASAAGPPIAATPPAYSTQYVAYSPQQFPNQPLVQHVPHYQSQHPHVYSPVIQGNARMMAPPTHAQPGLVSSSATQYGAHEQTHAMYVSTGSLAQQYAHPNATLHPHTPHPQPSATPTGQQQSQHGGSHPAPSPVQHHQHQAAQALHLASPQQQSAIYHAGLAPTPPSMTPASNTQSPQNSFPAAQQTVFTIHPSHVQPAYTNPPHMAHVPQAHVQSGMVPSHPTAHAPMMLMTTQPPGGPQAALAQSALQPIPVSTTAHFPYMTHPSVQAHHQQQL

SEQ ID NO: 67 - DNA, 4347 bp, Homo sapiens
  source 1..4347: mol_type = genomic DNA
agagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcaggtaaagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactga

SEQ ID NO: 68 - protein, 1155 aa, Homo sapiens
MSLKPQQQQQQQQQQQQQQQQQQQQQQQPPPAAANVRKPGGSGLLASPAAAPSPSSSSVSSSSATAPSSVVAATSGGGRPGLGRGRNSNKGLPQSTISFDGIYANMRMVHILTSVVGSKCEVQVKNGGIYEGVFKTYSPKCDLVLDAAHEKSTESSSGPKREEIMESILFKCSDFVVVQFKDMDSSYAKRDAFTDSAISAKVNGEHKEKDLEPWDAGELTANEELEALENDVSNGWDPNDMFRYNEENYGVVSTYDSSLSSYTVPLERDNSEEFLKREARANQLAEEIESSAQYKARVALENDDRSEEEKYTAVQRNSSEREGHSINTRENKYIPPGQRNREVISWGSGRQNSPRMGQPGSGSMPSRSTSHTSDFNPNSGSDQRVVNGGVPWPSPCPSPSSRPPSRYQSGPNSLPPRAATPTRPPSRPPSRPSRPPSHPSAHGSPAPVSTMPKRMSSEGPPRMSPKAQRHPRNHRVSAGRGSISSGLEFVSHNPPSEAATPPVARTSPSGGTWSSVVSGVPRLSPKTHRPRSPRQNSIGNTPSGPVLASPQAGIIPTEAVAMPIPAASPTPASPASNRAVTPSSEAKDSRLQDQRQNSPAGNKENIKPNETSPSFSKAENKGISPVVSEHRKQIDDLKKFKNDFRLQPSSTSESMDQLLNKNREGEKSRDLIKDKIEPSAKDSFIENSSSNCTSGSSKPNSPSISPSILSNTEHKRGPEVTSQGVQTSSPACKQEKDDKEEKKDAAEQVRKSTLNPNAKEFNPRSFSQPKPSTTPTSPRPQAQPSPSMVGHQQPTPVYTQPVCFAPNMMYPVPVSPGVQPLYPIPMTPMPVNQAKTYRAGKVPNMPQQRQDQHHQSAMMHPASAAGPPIAATPPAYSTQYVAYSPQQFPNQPLVQHVPHYQSQHPHVYSPVIQGNARMMAPPTHAQPGLVSSSATQYGAHEQTHAMYACPKLPYNKETSPSFYFAISTGSLAQQYAHPNATLHPHTPHPQPSATPTGQQQSQHGGSHPAPSPVQHHQHQAAQALHLASPQQQSAIYHAGLAPTPPSMTPASNTQSPQNSFPAAQQTVFTIHPSHVQPAYTNPPHMAHVPQAHVQSGMVPSHPTAHAPMMLMTTQPPGGPQAALAQSALQPIPVSTTAHFPYMTHPSVQAHHQQQL

SEQ ID NO: 69 - DNA, 3462 bp, Homo sapiens
  source 1..3462: mol_type = genomic DNA
atgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaa

SEQ ID NO: 70 - protein, 1153 aa, Homo sapiens; identical to SEQ ID NO: 62

(19) United States
(12) Patent Application Publication
(10) Pub. No.: US 2025/0179491 A1
(43) Pub. Date: Jun. 5, 2025

(54) ATXN2 RNA INTERFERENCE AGENTS

(71) Applicant: ELI LILLY AND COMPANY, Indianapolis, IN (US)

(72) Inventors: Andrew Peter McCARTHY, Arlington, MA (US); Hiroaki TANI, Indianapolis, IN (US); Jeremy S. YORK, Noblesville, IN (US)

(21) Appl. No.: 18/875,279

(22) PCT Filed: Jun. 21, 2023
(86) PCT No.: PCT/US2023/068805

§ 371 Date: Dec. 16, 2024

Related U.S. Application Data
(60) Provisional application No. 63/355,305, filed on Jun. 24, 2022.

(51) Int. Cl. C12N 15/113

(52) U.S. Cl. CPC C12N 15/113; C12N 2310/14; C12N 2310/31; C12N 2310/321; C12N 2310/351

(57) ABSTRACT

Provided herein are ATXN2 RNAi agents and compositions comprising an ATXN2 RNAi agent. Also provided herein are methods of using the ATXN2 RNAi agents or compositions comprising an ATXN2 RNAi agent for reducing ATXN2 expression, and/or treating ATXN2-associated neurological disease in a subject.

### SEQUENCE LISTING

The present application is being filed along with a Sequence Listing in ST.26 XML format. The Sequence Listing is provided as a file titled “30344v2” created 7 Jun. 2023 and is 412 kilobytes in size. The Sequence Listing information in the ST.26 XML format is incorporated herein by reference in its entirety.

### BACKGROUND

Ataxin-2 is encoded by the gene ATXN2 (also known as ATX2, SCA2, TNRC13). The ATXN2 gene includes CAG trinucleotide repeats, which result in a polyglutamine (polyQ) stretch in the N-terminal region of the Ataxin-2 protein. More than 90% of normal individuals have an ATXN2 allele with 22 polyQ repeats. CAG expansions of more than 22 repeats in ATXN2 are associated with certain neurodegenerative diseases.

Spinocerebellar ataxia 2 (SCA2) is caused by CAG expansion of 31 repeats or more in ATXN-2. The most common SCA2-associated ATXN2 alleles have 37-39 CAG repeats; the longer CAG repeat expansions are associated with earlier onset of SCA2. SCA2 is an autosomal dominant neurodegenerative disease characterized by progressive degeneration of neurons in the cerebellum, brain stem, and/or spinal cord. Patients with SCA2 show progressive incoordination of gait and often poor coordination of hands, speech and eye movements, likely due to cerebellum degeneration with variable involvement of the brainstem and spinal cord. Moderate CAG expansion (23 or more repeats but below the threshold for SCA2) in the ATXN2 gene is also associated with amyotrophic lateral sclerosis (ALS).

RNA interference (RNAi) is a highly conserved regulatory mechanism in which RNA molecules are involved in sequence-specific suppression of gene expression by double-stranded RNA molecules (dsRNA) (Fire et al., Nature 391:806-811, 1998).

There remains a need for therapeutic agents that can inhibit or adjust the expression of ATXN2 for treating ATXN2-associated neurological diseases such as SCA2 or ALS, e.g., by utilizing RNAi.

### SUMMARY OF INVENTION

Provided herein are ATXN2 RNAi agents and compositions comprising an ATXN2 RNAi agent. Also provided herein are methods of using the ATXN2 RNAi agents or compositions comprising an ATXN2 RNAi agent for reducing ATXN2 expression, and/or treating ATXN2-associated neurological diseases in a subject.

In one aspect, provided herein are ATXN2 RNAi agents comprising a sense strand and an antisense strand, wherein the sense strand and the antisense strand form a duplex, wherein the sense strand and the antisense strand comprise a pair of nucleic acid sequences selected from the group consisting of:
(a) the sense strand comprises a first nucleic acid sequence having at least 95% sequence identity to SEQ ID NO: 1, and the antisense strand comprises a second nucleic acid sequence having at least 95% sequence identity to SEQ ID NO: 2;
(b) the sense strand comprises a first nucleic acid sequence having at least 95% sequence identity to SEQ ID NO: 3, and the antisense strand comprises a second nucleic acid sequence having at least 95% sequence identity to SEQ ID NO: 4;
(c) the sense strand comprises a first nucleic acid sequence having at least 95% sequence identity to SEQ ID NO: 5, and the antisense strand comprises a second nucleic acid sequence having at least 95% sequence identity to SEQ ID NO: 6;
(d) the sense strand comprises a first nucleic acid sequence having at least 95% sequence identity to SEQ ID NO: 7, and the antisense strand comprises a second nucleic acid sequence having at least 95% sequence identity to SEQ ID NO: 8;
(e) the sense strand comprises a first nucleic acid sequence having at least 95% sequence identity to SEQ ID NO: 9, and the antisense strand comprises a second nucleic acid sequence having at least 95% sequence identity to SEQ ID NO: 10;
(f) the sense strand comprises a first nucleic acid sequence having at least 95% sequence identity to SEQ ID NO: 11, and the antisense strand comprises a second nucleic acid sequence having at least 95% sequence identity to SEQ ID NO: 12;
(g) the sense strand comprises a first nucleic acid sequence having at least 95% sequence identity to SEQ ID NO: 13, and the antisense strand comprises a second nucleic acid sequence having at least 95% sequence identity to SEQ ID NO: 14;
(h) the sense strand comprises a first nucleic acid sequence having at least 95% sequence identity to SEQ ID NO: 15, and the antisense strand comprises a second nucleic acid sequence having at least 95% sequence identity to SEQ ID NO: 16;
(i) the sense strand comprises a first nucleic acid sequence having at least 95% sequence identity to SEQ ID NO: 17, and the antisense strand comprises a second nucleic acid sequence having at least 95% sequence identity to SEQ ID NO: 18;
(j) the sense strand comprises a first nucleic acid sequence having at least 95% sequence identity to SEQ ID NO: 19, and the antisense strand comprises a second nucleic acid sequence having at least 95% sequence identity to SEQ ID NO: 20;
(k) the sense strand comprises a first nucleic acid sequence having at least 95% sequence identity to SEQ ID NO: 21, and the antisense strand comprises a second nucleic acid sequence having at least 95% sequence identity to SEQ ID NO: 22;
(l) the sense strand comprises a first nucleic acid sequence having at least 95% sequence identity to SEQ ID NO: 23, and the antisense strand comprises a second nucleic acid sequence having at least 95% sequence identity to SEQ ID NO: 24;
(m) the sense strand comprises a first nucleic acid sequence having at least 95% sequence identity to SEQ ID NO: 25, and the antisense strand comprises a second nucleic acid sequence having at least 95% sequence identity to SEQ ID NO: 26;
(n) the sense strand comprises a first nucleic acid sequence having at least 95% sequence identity to SEQ ID NO: 27, and the antisense strand comprises a second nucleic acid sequence having at least 95% sequence identity to SEQ ID NO: 28; and
(o) the sense strand comprises a first nucleic acid sequence having at least 95% sequence identity to SEQ ID NO: 29, and the antisense strand comprises a second nucleic acid sequence having at least 95% sequence identity to SEQ ID NO: 30;
   wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand and the antisense strand of the ATXN2 RNAi agent described herein comprise a pair of nucleic acid sequences selected from the group consisting of:
(a) the sense strand comprises a first nucleic acid sequence of SEQ ID NO: 1, and the antisense strand comprises a second nucleic acid sequence of SEQ ID NO: 2;
(b) the sense strand comprises a first nucleic acid sequence of SEQ ID NO: 3, and the antisense strand comprises a second nucleic acid sequence of SEQ ID NO: 4;
(c) the sense strand comprises a first nucleic acid sequence of SEQ ID NO: 5, and the antisense strand comprises a second nucleic acid sequence of SEQ ID NO: 6;
(d) the sense strand comprises a first nucleic acid sequence of SEQ ID NO: 7, and the antisense strand comprises a second nucleic acid sequence of SEQ ID NO: 8;
(e) the sense strand comprises a first nucleic acid sequence of SEQ ID NO: 9, and the antisense strand comprises a second nucleic acid sequence of SEQ ID NO: 10;
(f) the sense strand comprises a first nucleic acid sequence of SEQ ID NO: 11, and the antisense strand comprises a second nucleic acid sequence of SEQ ID NO: 12;
(g) the sense strand comprises a first nucleic acid sequence of SEQ ID NO: 13, and the antisense strand comprises a second nucleic acid sequence of SEQ ID NO: 14;
(h) the sense strand comprises a first nucleic acid sequence of SEQ ID NO: 15, and the antisense strand comprises a second nucleic acid sequence of SEQ ID NO: 16;
(i) the sense strand comprises a first nucleic acid sequence of SEQ ID NO: 17, and the antisense strand comprises a second nucleic acid sequence of SEQ ID NO: 18;
(j) the sense strand comprises a first nucleic acid sequence of SEQ ID NO: 19, and the antisense strand comprises a second nucleic acid sequence of SEQ ID NO: 20;
(k) the sense strand comprises a first nucleic acid sequence of SEQ ID NO: 21, and the antisense strand comprises a second nucleic acid sequence of SEQ ID NO: 22;
(l) the sense strand comprises a first nucleic acid sequence of SEQ ID NO: 23, and the antisense strand comprises a second nucleic acid sequence of SEQ ID NO: 24;
(m) the sense strand comprises a first nucleic acid sequence of SEQ ID NO: 25, and the antisense strand comprises a second nucleic acid sequence of SEQ ID NO: 26;
(n) the sense strand comprises a first nucleic acid sequence of SEQ ID NO: 27, and the antisense strand comprises a second nucleic acid sequence of SEQ ID NO: 28; and
(o) the sense strand comprises a first nucleic acid sequence of SEQ ID NO: 29, and the antisense strand comprises a second nucleic acid sequence of SEQ ID NO: 30,
   wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the ATXN2 RNAi agent described herein can comprise a sense strand that comprises a sequence that has 1, 2, or 3 differences from SEQ ID NO: 1, 3, 5, 7, 9, 11, 13, 15, 17, 19, 21, 23, 25, 27, 29. In some embodiments, the ATXN2 RNAi agent described herein can comprise a antisense strand that comprises a sequence that has 1, 2, or 3 differences from SEQ ID NO: 2, 4, 6, 8, 10, 12, 14, 16, 18, 20, 22, 24, 26, 28, 30.

The ATXN2 RNAi agents described herein may include modifications. The modifications can be made to one or more nucleotides of the sense strand and/or antisense strand or to the internucleotide linkages. In some embodiments, one or more nucleotides of the sense strand are modified nucleotides. In some embodiments, each nucleotide of the sense strand is a modified nucleotide. In some embodiments, one or more nucleotides of the antisense strand are modified nucleotides. In some embodiments, each nucleotide of the antisense strand is a modified nucleotide. In some embodiments, the modified nucleotide is a 2′-fluoro modified nucleotide, 2′-O-methyl modified nucleotide, or 2′-O-alkyl modified nucleotide. In some embodiments, the sense strand has four 2′-fluoro modified nucleotides at positions 7, 9, 10, and 11 from the 5′ end of the sense strand. In some embodiments, nucleotides at positions other than positions 7, 9, 10, and 11 of the sense strand are 2′-O-methyl modified nucleotides. In some embodiments, the antisense strand has four 2′-fluoro modified nucleotides at positions 2, 6, 14, and 16 from the 5′ end of the antisense strand. In some embodiments, nucleotides at positions other than positions 2, 6, 14 and 16 of the antisense strand are 2′-O-methyl modified nucleotides. In some embodiments, the first nucleotide from the 5′ end of the antisense strand is a modified nucleotide that has a phosphate analog, e.g., a 5′-vinylphosphonate. In some embodiments, the sense strand has an abasic moiety or inverted abasic moiety, e.g., at position 9, 10 or 11. In some embodiments, the sense strand and the antisense strand have one or more modified internucleotide linkages, e.g., phosphorothioate linkage. In some embodiments, the sense strand has four or five phosphorothioate linkages. In some embodiments, the antisense strand has four or five phosphorothioate linkages. In some embodiments, the sense strand has four phosphorothioate linkages and the antisense strand has four phosphorothioate linkages.

In some embodiments, provided herein are ATXN2 RNAi agents comprising a sense strand and an antisense strand, wherein the sense strand and the antisense strand form a duplex, wherein the sense strand and the antisense strand comprise a pair of nucleic acid sequences selected from the group consisting of:
(a) the sense strand comprises a first nucleic acid sequence of SEQ ID NO: 31, and the antisense strand comprises a second nucleic acid sequence of SEQ ID NO: 32;
(b) the sense strand comprises a first nucleic acid sequence of SEQ ID NO: 33, and the antisense strand comprises a second nucleic acid sequence of SEQ ID NO: 34;
(c) the sense strand comprises a first nucleic acid sequence of SEQ ID NO: 35, and the antisense strand comprises a second nucleic acid sequence of SEQ ID NO: 36;
(d) the sense strand comprises a first nucleic acid sequence of SEQ ID NO: 37, and the antisense strand comprises a second nucleic acid sequence of SEQ ID NO: 38;
(e) the sense strand comprises a first nucleic acid sequence of SEQ ID NO: 39, and the antisense strand comprises a second nucleic acid sequence of SEQ ID NO: 40;
(f) the sense strand comprises a first nucleic acid sequence of SEQ ID NO: 41, and the antisense strand comprises a second nucleic acid sequence of SEQ ID NO: 42;
(g) the sense strand comprises a first nucleic acid sequence of SEQ ID NO: 43, and the antisense strand comprises a second nucleic acid sequence of SEQ ID NO: 44;
(h) the sense strand comprises a first nucleic acid sequence of SEQ ID NO: 45, and the antisense strand comprises a second nucleic acid sequence of SEQ ID NO: 46;
(i) the sense strand comprises a first nucleic acid sequence of SEQ ID NO: 47, and the antisense strand comprises a second nucleic acid sequence of SEQ ID NO: 48;
(j) the sense strand comprises a first nucleic acid sequence of SEQ ID NO: 49, and the antisense strand comprises a second nucleic acid sequence of SEQ ID NO: 50;
(k) the sense strand comprises a first nucleic acid sequence of SEQ ID NO: 51, and the antisense strand comprises a second nucleic acid sequence of SEQ ID NO: 52;
(l) the sense strand comprises a first nucleic acid sequence of SEQ ID NO: 53, and the antisense strand comprises a second nucleic acid sequence of SEQ ID NO: 54;
(m) the sense strand comprises a first nucleic acid sequence of SEQ ID NO: 55, and the antisense strand comprises a second nucleic acid sequence of SEQ ID NO: 56;
(n) the sense strand comprises a first nucleic acid sequence of SEQ ID NO: 57, and the antisense strand comprises a second nucleic acid sequence of SEQ ID NO: 58; and
(o) the sense strand comprises a first nucleic acid sequence of SEQ ID NO: 59, and the antisense strand comprises a second nucleic acid sequence of SEQ ID NO: 60.

In some embodiments, the ATXN2 RNAi agent described herein can comprise a sense strand that comprises a sequence that has 1, 2, or 3 differences from SEQ ID NO: 31, 33, 35, 37, 39, 41, 43, 45, 47, 49, 51, 53, 55, 57, 59. In some embodiments, the ATXN2 RNAi agent described herein can comprise a antisense strand that comprises a sequence that has 1, 2, or 3 differences from SEQ ID NO: 32, 34, 36, 38, 40, 42, 44, 46, 48, 50, 52, 54, 56, 58, 60.

In some embodiments, provided herein are ATXN2 RNAi agents comprising a sense strand and an antisense strand, wherein the sense strand and the antisense strand form a duplex, wherein the sense strand and the antisense strand have a pair of nucleic acid sequences selected from the group consisting of:
(a) the sense strand consists of a first nucleic acid sequence of SEQ ID NO: 31, and the antisense strand consists of a second nucleic acid sequence of SEQ ID NO: 32;
(b) the sense strand consists of a first nucleic acid sequence of SEQ ID NO: 33, and the antisense strand consists of a second nucleic acid sequence of SEQ ID NO: 34;
(c) the sense strand consists of a first nucleic acid sequence of SEQ ID NO: 35, and the antisense strand consists of a second nucleic acid sequence of SEQ ID NO: 36;
(d) the sense strand consists of a first nucleic acid sequence of SEQ ID NO: 37, and the antisense strand consists of a second nucleic acid sequence of SEQ ID NO: 38;
(e) the sense strand consists of a first nucleic acid sequence of SEQ ID NO: 39, and the antisense strand consists of a second nucleic acid sequence of SEQ ID NO: 40;
(f) the sense strand consists of a first nucleic acid sequence of SEQ ID NO: 41, and the antisense strand consists of a second nucleic acid sequence of SEQ ID NO: 42;
(g) the sense strand consists of a first nucleic acid sequence of SEQ ID NO: 43, and the antisense strand consists of a second nucleic acid sequence of SEQ ID NO: 44;
(h) the sense strand consists of a first nucleic acid sequence of SEQ ID NO: 45, and the antisense strand consists of a second nucleic acid sequence of SEQ ID NO: 46;
(i) the sense strand consists of a first nucleic acid sequence of SEQ ID NO: 47, and the antisense strand consists of a second nucleic acid sequence of SEQ ID NO: 48;
(j) the sense strand consists of a first nucleic acid sequence of SEQ ID NO: 49, and the antisense strand consists of a second nucleic acid sequence of SEQ ID NO: 50;
(k) the sense strand consists of a first nucleic acid sequence of SEQ ID NO: 51, and the antisense strand consists of a second nucleic acid sequence of SEQ ID NO: 52;
(l) the sense strand consists of a first nucleic acid sequence of SEQ ID NO: 53, and the antisense strand consists of a second nucleic acid sequence of SEQ ID NO: 54;
(m) the sense strand consists of a first nucleic acid sequence of SEQ ID NO: 55, and the antisense strand consists of a second nucleic acid sequence of SEQ ID NO: 56;
(n) the sense strand consists of a first nucleic acid sequence of SEQ ID NO: 57, and the antisense strand consists of a second nucleic acid sequence of SEQ ID NO: 58; and
(o) the sense strand consists of a first nucleic acid sequence of SEQ ID NO: 59, and the antisense strand consists of a second nucleic acid sequence of SEQ ID NO: 60.

In some embodiments, the sense strand of the ATXN2 RNAi agent has a delivery moiety conjugated to the 3′ end of the sense strand. In some embodiments, the sense strand of the ATXN2 RNAi agent has a delivery moiety conjugated to a nucleotide of the sense strand. In some embodiments, the delivery moiety is α-tocopherol, cholesterol, or palmitic acid. In some embodiments, the delivery moiety is conjugated to the 3′ end of the sense stand via a linker, e.g., a linker of Table 5.

In a further aspect, provided herein are ATXN2 RNAi agents of Formula (I): R-L-D, wherein R is a double stranded RNA (dsRNA) comprising a sense stand and an antisense strand, wherein the sense strand and the antisense strand form a duplex; wherein D is a delivery means for delivering the dsRNA into a cell; and wherein L is a linking means for linking the dsRNA to the delivery means, or optionally absent, wherein the sense strand and the antisense strand comprises a pair of nucleic acid sequences selected from the group consisting of:
(a) the sense strand comprises a first nucleic acid sequence of SEQ ID NO: 1, and the antisense strand comprises a second nucleic acid sequence of SEQ ID NO: 2;
(b) the sense strand comprises a first nucleic acid sequence of SEQ ID NO: 3, and the antisense strand comprises a second nucleic acid sequence of SEQ ID NO: 4;
(c) the sense strand comprises a first nucleic acid sequence of SEQ ID NO: 5, and the antisense strand comprises a second nucleic acid sequence of SEQ ID NO: 6;
(d) the sense strand comprises a first nucleic acid sequence of SEQ ID NO: 7, and the antisense strand comprises a second nucleic acid sequence of SEQ ID NO: 8;
(e) the sense strand comprises a first nucleic acid sequence of SEQ ID NO: 9, and the antisense strand comprises a second nucleic acid sequence of SEQ ID NO: 10;
(f) the sense strand comprises a first nucleic acid sequence of SEQ ID NO: 11, and the antisense strand comprises a second nucleic acid sequence of SEQ ID NO: 12;
(g) the sense strand comprises a first nucleic acid sequence of SEQ ID NO: 13, and the antisense strand comprises a second nucleic acid sequence of SEQ ID NO: 14;
(h) the sense strand comprises a first nucleic acid sequence of SEQ ID NO: 15, and the antisense strand comprises a second nucleic acid sequence of SEQ ID NO: 16;
(i) the sense strand comprises a first nucleic acid sequence of SEQ ID NO: 17, and the antisense strand comprises a second nucleic acid sequence of SEQ ID NO: 18;
(j) the sense strand comprises a first nucleic acid sequence of SEQ ID NO: 19, and the antisense strand comprises a second nucleic acid sequence of SEQ ID NO: 20;
(k) the sense strand comprises a first nucleic acid sequence of SEQ ID NO: 21, and the antisense strand comprises a second nucleic acid sequence of SEQ ID NO: 22;
(l) the sense strand comprises a first nucleic acid sequence of SEQ ID NO: 23, and the antisense strand comprises a second nucleic acid sequence of SEQ ID NO: 24;
(m) the sense strand comprises a first nucleic acid sequence of SEQ ID NO: 25, and the antisense strand comprises a second nucleic acid sequence of SEQ ID NO: 26;
(n) the sense strand comprises a first nucleic acid sequence of SEQ ID NO: 27, and the antisense strand comprises a second nucleic acid sequence of SEQ ID NO: 28; and
(o) the sense strand comprises a first nucleic acid sequence of SEQ ID NO: 29, and the antisense strand comprises a second nucleic acid sequence of SEQ ID NO: 30,
   wherein optionally one or more nucleotides of the sense strand and the antisense strand are independently modified nucleotides, and wherein optionally one or more internucleotide linkages of the sense strand and the antisense strand are modified internucleotide linkages.

In some embodiments, provided herein are ATXN2 RNAi agents of Formula (I): R-L-D, wherein R is a dsRNA comprising a sense stand and an antisense strand, wherein the sense strand and the antisense strand form a duplex; wherein D is a delivery moiety; and wherein L is a linker or optionally absent, wherein the sense strand and the antisense strand comprise a pair of nucleic acid sequences selected from the group consisting of:
(a) the sense strand comprises a first nucleic acid sequence of SEQ ID NO: 1, and the antisense strand comprises a second nucleic acid sequence of SEQ ID NO: 2;
(b) the sense strand comprises a first nucleic acid sequence of SEQ ID NO: 3, and the antisense strand comprises a second nucleic acid sequence of SEQ ID NO: 4;
(c) the sense strand comprises a first nucleic acid sequence of SEQ ID NO: 5, and the antisense strand comprises a second nucleic acid sequence of SEQ ID NO: 6;
(d) the sense strand comprises a first nucleic acid sequence of SEQ ID NO: 7, and the antisense strand comprises a second nucleic acid sequence of SEQ ID NO: 8;
(e) the sense strand comprises a first nucleic acid sequence of SEQ ID NO: 9, and the antisense strand comprises a second nucleic acid sequence of SEQ ID NO: 10;
(f) the sense strand comprises a first nucleic acid sequence of SEQ ID NO: 11, and the antisense strand comprises a second nucleic acid sequence of SEQ ID NO: 12;
(g) the sense strand comprises a first nucleic acid sequence of SEQ ID NO: 13, and the antisense strand comprises a second nucleic acid sequence of SEQ ID NO: 14;
(h) the sense strand comprises a first nucleic acid sequence of SEQ ID NO: 15, and the antisense strand comprises a second nucleic acid sequence of SEQ ID NO: 16;
(i) the sense strand comprises a first nucleic acid sequence of SEQ ID NO: 17, and the antisense strand comprises a second nucleic acid sequence of SEQ ID NO: 18;
(j) the sense strand comprises a first nucleic acid sequence of SEQ ID NO: 19, and the antisense strand comprises a second nucleic acid sequence of SEQ ID NO: 20;
(k) the sense strand comprises a first nucleic acid sequence of SEQ ID NO: 21, and the antisense strand comprises a second nucleic acid sequence of SEQ ID NO: 22;
(l) the sense strand comprises a first nucleic acid sequence of SEQ ID NO: 23, and the antisense strand comprises a second nucleic acid sequence of SEQ ID NO: 24;
(m) the sense strand comprises a first nucleic acid sequence of SEQ ID NO: 25, and the antisense strand comprises a second nucleic acid sequence of SEQ ID NO: 26;
(n) the sense strand comprises a first nucleic acid sequence of SEQ ID NO: 27, and the antisense strand comprises a second nucleic acid sequence of SEQ ID NO: 28; and
(o) the sense strand comprises a first nucleic acid sequence of SEQ ID NO: 29, and the antisense strand comprises a second nucleic acid sequence of SEQ ID NO: 30,
   wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand and the antisense strand in RNAi agents of Formula (I) comprise a pair of nucleic acid sequences selected from the group consisting of:
(a) the sense strand comprises a first nucleic acid sequence of SEQ ID NO: 31, and the antisense strand comprises a second nucleic acid sequence of SEQ ID NO: 32;
(b) the sense strand comprises a first nucleic acid sequence of SEQ ID NO: 33, and the antisense strand comprises a second nucleic acid sequence of SEQ ID NO: 34;
(c) the sense strand comprises a first nucleic acid sequence of SEQ ID NO: 35, and the antisense strand comprises a second nucleic acid sequence of SEQ ID NO: 36;
(d) the sense strand comprises a first nucleic acid sequence of SEQ ID NO: 37, and the antisense strand comprises a second nucleic acid sequence of SEQ ID NO: 38;
(e) the sense strand comprises a first nucleic acid sequence of SEQ ID NO: 39, and the antisense strand comprises a second nucleic acid sequence of SEQ ID NO: 40;
(f) the sense strand comprises a first nucleic acid sequence of SEQ ID NO: 41, and the antisense strand comprises a second nucleic acid sequence of SEQ ID NO: 42;
(g) the sense strand comprises a first nucleic acid sequence of SEQ ID NO: 43, and the antisense strand comprises a second nucleic acid sequence of SEQ ID NO: 44;
(h) the sense strand comprises a first nucleic acid sequence of SEQ ID NO: 45, and the antisense strand comprises a second nucleic acid sequence of SEQ ID NO: 46;
(i) the sense strand comprises a first nucleic acid sequence of SEQ ID NO: 47, and the antisense strand comprises a second nucleic acid sequence of SEQ ID NO: 48;
(j) the sense strand comprises a first nucleic acid sequence of SEQ ID NO: 49, and the antisense strand comprises a second nucleic acid sequence of SEQ ID NO: 50;
(k) the sense strand comprises a first nucleic acid sequence of SEQ ID NO: 51, and the antisense strand comprises a second nucleic acid sequence of SEQ ID NO: 52;
(l) the sense strand comprises a first nucleic acid sequence of SEQ ID NO: 53, and the antisense strand comprises a second nucleic acid sequence of SEQ ID NO: 54;
(m) the sense strand comprises a first nucleic acid sequence of SEQ ID NO: 55, and the antisense strand comprises a second nucleic acid sequence of SEQ ID NO: 56;
(n) the sense strand comprises a first nucleic acid sequence of SEQ ID NO: 57, and the antisense strand comprises a second nucleic acid sequence of SEQ ID NO: 58;
(o) the sense strand comprises a first nucleic acid sequence of SEQ ID NO: 59, and the antisense strand comprises a second nucleic acid sequence of SEQ ID NO: 60.

In another aspect, provided herein are pharmaceutical compositions comprising an ATXN2 RNAi agent described herein and a pharmaceutically acceptable carrier. Also provided herein are pharmaceutical compositions comprising a means for reducing ATXN2 expression in a cell and a pharmaceutically acceptable carrier.

In another aspect, provided herein are methods of reducing ATXN2 expression in a patient in need thereof, and such method comprises administering to the patient an effective amount of an ATXN2 RNAi agent or a pharmaceutical composition described herein.

In another aspect, provided herein are methods of treating an ATXN2-associated neurological disease in a patient in need thereof, and such method comprises administering to the patient an effective amount of the ATXN2 RNAi agent or a pharmaceutical composition described herein.

Also provided herein are methods of reducing ATXN2 expression in a cell (e.g., a neuron), and such methods can include introducing an ATXN2 RNAi agent described herein into the cell; and incubating the cell for a time sufficient for degradation of ATXN2 mRNA, thereby reducing ATXN2 expression in the cell.

In another aspect, provided herein are ATXN2 RNAi agents or pharmaceutical compositions comprising an ATXN2 RNAi agent for use in reducing ATXN2 expression. Also provided herein are ATXN2 RNAi agents or the pharmaceutical composition comprising an ATXN2 RNAi agent for use in a therapy. Also provided are ATXN2 RNAi agents or pharmaceutical compositions comprising an ATXN2 RNAi agent for use in the treatment of an ATXN2-associated neurological disease. Also provided herein are uses of ATXN2 RNAi agents in the manufacture of a medicament for the treatment of an ATXN2-associated neurological disease.

### DETAILED DESCRIPTION

Provided herein are ATXN2 RNAi agents and compositions comprising an ATXN2 RNAi agent. Also provided herein are methods of using the ATXN2 RNAi agents or compositions comprising an ATXN2 RNAi agent for reducing ATXN2 expression, and/or treating ATXN2-associated neurological disease in a subject.

In some embodiments, provided herein are ATXN2 RNAi agents comprising a sense strand and an antisense strand, and the sense strand and the antisense strand form a duplex. The antisense strand is complementary to a region of ATXN2 mRNA. In a further embodiment, the sense strand and the antisense strand are each 15-30 nucleotides in length, e.g., 20-25 nucleotides in length. In some embodiments, provided herein are ATXN2 RNAi agents comprising a sense strand of 21 nucleotides and an antisense strand of 23 nucleotides. In some embodiments, the sense strand and antisense strand of the ATXN2 RNAi agent may have overhangs at either the 5′ end or the 3′ end (i.e., 5′ overhang or 3′ overhang). For example, the sense strand and the antisense strand may have 5′ or 3′ overhangs of 1 to 5 nucleotides or 1 to 3 nucleotides. In some embodiments, the antisense strand comprises a 3′ overhang of two nucleotides. In some embodiments, the sense strand and antisense strand sequences of the ATXN2 RNAi agents are provided in Table 1.

Provided herein are ATXN2 RNAi agents comprising a sense strand and an antisense strand, wherein the sense strand and the antisense strand form a duplex, wherein the sense strand and the antisense strand comprise a pair of nucleic acid sequences selected from the group consisting of:
(a) the sense strand comprises a first nucleic acid sequence having at least 90% sequence identity to SEQ ID NO: 1, and the antisense strand comprises a second nucleic acid sequence having at least 90% sequence identity to SEQ ID NO: 2;
(b) the sense strand comprises a first nucleic acid sequence having at least 90% sequence identity to SEQ ID NO: 3, and the antisense strand comprises a second nucleic acid sequence having at least 90% sequence identity to SEQ ID NO: 4;
(c) the sense strand comprises a first nucleic acid sequence having at least 90% sequence identity to SEQ ID NO: 5, and the antisense strand comprises a second nucleic acid sequence having at least 90% sequence identity to SEQ ID NO: 6;
(d) the sense strand comprises a first nucleic acid sequence having at least 90% sequence identity to SEQ ID NO: 7, and the antisense strand comprises a second nucleic acid sequence having at least 90% sequence identity to SEQ ID NO: 8;
(e) the sense strand comprises a first nucleic acid sequence having at least 90% sequence identity to SEQ ID NO: 9, and the antisense strand comprises a second nucleic acid sequence having at least 90% sequence identity to SEQ ID NO: 10;
(f) the sense strand comprises a first nucleic acid sequence having at least 90% sequence identity to SEQ ID NO: 11, and the antisense strand comprises a second nucleic acid sequence having at least 90% sequence identity to SEQ ID NO: 12;
(g) the sense strand comprises a first nucleic acid sequence having at least 90% sequence identity to SEQ ID NO: 13, and the antisense strand comprises a second nucleic acid sequence having at least 90% sequence identity to SEQ ID NO: 14;
(h) the sense strand comprises a first nucleic acid sequence having at least 90% sequence identity to SEQ ID NO: 15, and the antisense strand comprises a second nucleic acid sequence having at least 90% sequence identity to SEQ ID NO: 16;
(i) the sense strand comprises a first nucleic acid sequence having at least 90% sequence identity to SEQ ID NO: 17, and the antisense strand comprises a second nucleic acid sequence having at least 90% sequence identity to SEQ ID NO: 18;
(j) the sense strand comprises a first nucleic acid sequence having at least 90% sequence identity to SEQ ID NO: 19, and the antisense strand comprises a second nucleic acid sequence having at least 90% sequence identity to SEQ ID NO: 20;
(k) the sense strand comprises a first nucleic acid sequence having at least 90% sequence identity to SEQ ID NO: 21, and the antisense strand comprises a second nucleic acid sequence having at least 90% sequence identity to SEQ ID NO: 22;
(l) the sense strand comprises a first nucleic acid sequence having at least 90% sequence identity to SEQ ID NO: 23, and the antisense strand comprises a second nucleic acid sequence having at least 90% sequence identity to SEQ ID NO: 24;
(m) the sense strand comprises a first nucleic acid sequence having at least 90% sequence identity to SEQ ID NO: 25, and the antisense strand comprises a second nucleic acid sequence having at least 90% sequence identity to SEQ ID NO: 26;
(n) the sense strand comprises a first nucleic acid sequence having at least 90% sequence identity to SEQ ID NO: 27, and the antisense strand comprises a second nucleic acid sequence having at least 90% sequence identity to SEQ ID NO: 28; and
(o) the sense strand comprises a first nucleic acid sequence having at least 90% sequence identity to SEQ ID NO: 29, and the antisense strand comprises a second nucleic acid sequence having at least 90% sequence identity to SEQ ID NO: 30;
   wherein optionally one or more nucleotides of the sense strand and the antisense strand are independently modified nucleotides, and wherein optionally one or more internucleotide linkages of the sense strand and the antisense strand are modified internucleotide linkages.

Provided herein are ATXN2 RNAi agents comprising a sense strand and an antisense strand, wherein the sense strand and the antisense strand form a duplex, wherein the sense strand and the antisense strand comprise a pair of nucleic acid sequences selected from the group consisting of:
(a) the sense strand comprises a first nucleic acid sequence having at least 95% sequence identity to SEQ ID NO: 1, and the antisense strand comprises a second nucleic acid sequence having at least 95% sequence identity to SEQ ID NO: 2;
(b) the sense strand comprises a first nucleic acid sequence having at least 95% sequence identity to SEQ ID NO: 3, and the antisense strand comprises a second nucleic acid sequence having at least 95% sequence identity to SEQ ID NO: 4;
(c) the sense strand comprises a first nucleic acid sequence having at least 95% sequence identity to SEQ ID NO: 5, and the antisense strand comprises a second nucleic acid sequence having at least 95% sequence identity to SEQ ID NO: 6;
(d) the sense strand comprises a first nucleic acid sequence having at least 95% sequence identity to SEQ ID NO: 7, and the antisense strand comprises a second nucleic acid sequence having at least 95% sequence identity to SEQ ID NO: 8;
(e) the sense strand comprises a first nucleic acid sequence having at least 95% sequence identity to SEQ ID NO: 9, and the antisense strand comprises a second nucleic acid sequence having at least 95% sequence identity to SEQ ID NO: 10;
(f) the sense strand comprises a first nucleic acid sequence having at least 95% sequence identity to SEQ ID NO: 11, and the antisense strand comprises a second nucleic acid sequence having at least 95% sequence identity to SEQ ID NO: 12;
(g) the sense strand comprises a first nucleic acid sequence having at least 95% sequence identity to SEQ ID NO: 13, and the antisense strand comprises a second nucleic acid sequence having at least 95% sequence identity to SEQ ID NO: 14;
(h) the sense strand comprises a first nucleic acid sequence having at least 95% sequence identity to SEQ ID NO: 15, and the antisense strand comprises a second nucleic acid sequence having at least 95% sequence identity to SEQ ID NO: 16;
(i) the sense strand comprises a first nucleic acid sequence having at least 95% sequence identity to SEQ ID NO: 17, and the antisense strand comprises a second nucleic acid sequence having at least 95% sequence identity to SEQ ID NO: 18;
(j) the sense strand comprises a first nucleic acid sequence having at least 95% sequence identity to SEQ ID NO: 19, and the antisense strand comprises a second nucleic acid sequence having at least 95% sequence identity to SEQ ID NO: 20;
(k) the sense strand comprises a first nucleic acid sequence having at least 95% sequence identity to SEQ ID NO: 21, and the antisense strand comprises a second nucleic acid sequence having at least 95% sequence identity to SEQ ID NO: 22;
(l) the sense strand comprises a first nucleic acid sequence having at least 95% sequence identity to SEQ ID NO: 23, and the antisense strand comprises a second nucleic acid sequence having at least 95% sequence identity to SEQ ID NO: 24;
(m) the sense strand comprises a first nucleic acid sequence having at least 95% sequence identity to SEQ ID NO: 25, and the antisense strand comprises a second nucleic acid sequence having at least 95% sequence identity to SEQ ID NO: 26;
(n) the sense strand comprises a first nucleic acid sequence having at least 95% sequence identity to SEQ ID NO: 27, and the antisense strand comprises a second nucleic acid sequence having at least 95% sequence identity to SEQ ID NO: 28; and
(o) the sense strand comprises a first nucleic acid sequence having at least 95% sequence identity to SEQ ID NO: 29, and the antisense strand comprises a second nucleic acid sequence having at least 95% sequence identity to SEQ ID NO: 30;
   wherein optionally one or more nucleotides of the sense strand and the antisense strand are independently modified nucleotides, and wherein optionally one or more internucleotide linkages of the sense strand and the antisense strand are modified internucleotide linkages.

TABLE 1
Nucleic Acid Sequences of ATXN2 RNAi Agents

| | | | | | Start |
|---|---|---|---|---|---|
| | | | | | position |
| | | | | | of |
| | | | | | antisense |
| | | | | | strand |
| | | | | | target |
| | | | | | region of |
| ATXN | | | | | human |
| 2 | | | | | ATXN2 |
| RNAi | Sense | SEQ | Antisense | SEQ | transcript |
| Agent | Strand | ID | Strand | ID | ENST00000 |
| No. | (5′ to 3′) | NO | (5′ to 3′) | NO | 608853.5 |
| 1 | CGAAACGUGAA | 1 | UCAUUAUUUCUU | 2 | 793 |
| | GAAAUAAUGA | | CACGUUUCGGC | | |
| 2 | CCUAAACGCAU | 3 | UUCUGAAGACAU | 4 | 1671 |
| | GUCUUCAGAA | | GCGUUUAGGCA | | |
| 3 | GAGAUGCUUUU | 5 | UAGAGUCAGUAA | 6 | 886 |
| | ACUGACUCUA | | AAGCAUCUCUU | | |
| 4 | AAACGUGAAGA | 7 | UUCCAUUAUUUC | 8 | 795 |
| | AAUAAUGGAA | | UUCACGUUUCG | | |
| 5 | GCUCCUGUCUC | 9 | UGGCAUAGUAGA | 10 | 1653 |
| | UACUAUGCCA | | GACAGGAGCUG | | |
| 6 | UGGUUCUCCAG | 11 | UAGACAGGAGCU | 12 | 1643 |
| | CUCCUGUCUA | | GGAGAACCAUG | | |
| 7 | GCCUAAACGCA | 13 | UCUGAAGACAUG | 14 | 1670 |
| | UGUCUUCAGA | | CGUUUAGGCAU | | |
| 8 | UUAUCAGUCUC | 15 | UGAGGAUGCUGA | 16 | 3008 |
| | AGCAUCCUCA | | GACUGAUAAUG | | |
| 9 | UCUCCAGCUCC | 17 | UGUAGAGACAGG | 18 | 1674 |
| | UGUCUCUACA | | AGCUGGAGAAC | | |
| 10 | GCUCAUGGUUC | 19 | UGGAGCUGGAGA | 20 | 1638 |
| | UCCAGCUCCA | | ACCAUGAGCAG | | |
| 11 | CAGUCAGGUCC | 21 | UAGAGAGUUGGG | 22 | 1539 |
| | CAACUCUCUA | | ACCUGACUGGU | | |
| 12 | GAGUAGUUAAU | 23 | UAACACCUCCAU | 24 | 1468 |
| | GGAGGUGUUA | | UAACUACUCUU | | |
| 13 | AGUCAGGUCCC | 25 | UAAGAGAGUUGG | 26 | 1540 |
| | AACUCUCUUA | | GACCUGACUGG | | |
| 14 | CCCACCUUCUC | 27 | UACUGGUAGCGA | 28 | 1523 |
| | GCUACCAGUA | | GAAGGUGGGCG | | |
| 15 | CAAGACAUAUA | 29 | UGUACUGCUCUA | 30 | 2819 |
| | GAGCAGUACA | | UAUGUCUUGGC | | |

In some embodiments, the sense strand and the antisense strand of the ATXN2 RNAi agent described herein comprise a pair of nucleic acid sequences selected from the group consisting of:
(a) the sense strand comprises a first nucleic acid sequence of SEQ ID NO: 1, and the antisense strand comprises a second nucleic acid sequence of SEQ ID NO: 2;
(b) the sense strand comprises a first nucleic acid sequence of SEQ ID NO: 3, and the antisense strand comprises a second nucleic acid sequence of SEQ ID NO: 4;
(c) the sense strand comprises a first nucleic acid sequence of SEQ ID NO: 5, and the antisense strand comprises a second nucleic acid sequence of SEQ ID NO: 6;
(d) the sense strand comprises a first nucleic acid sequence of SEQ ID NO: 7, and the antisense strand comprises a second nucleic acid sequence of SEQ ID NO: 8;
(e) the sense strand comprises a first nucleic acid sequence of SEQ ID NO: 9, and the antisense strand comprises a second nucleic acid sequence of SEQ ID NO: 10;
(f) the sense strand comprises a first nucleic acid sequence of SEQ ID NO: 11, and the antisense strand comprises a second nucleic acid sequence of SEQ ID NO: 12;
(g) the sense strand comprises a first nucleic acid sequence of SEQ ID NO: 13, and the antisense strand comprises a second nucleic acid sequence of SEQ ID NO: 14;
(h) the sense strand comprises a first nucleic acid sequence of SEQ ID NO: 15, and the antisense strand comprises a second nucleic acid sequence of SEQ ID NO: 16;
(i) the sense strand comprises a first nucleic acid sequence of SEQ ID NO: 17, and the antisense strand comprises a second nucleic acid sequence of SEQ ID NO: 18;
(j) the sense strand comprises a first nucleic acid sequence of SEQ ID NO: 19, and the antisense strand comprises a second nucleic acid sequence of SEQ ID NO: 20;
(k) the sense strand comprises a first nucleic acid sequence of SEQ ID NO: 21, and the antisense strand comprises a second nucleic acid sequence of SEQ ID NO: 22;
(l) the sense strand comprises a first nucleic acid sequence of SEQ ID NO: 23, and the antisense strand comprises a second nucleic acid sequence of SEQ ID NO: 24;
(m) the sense strand comprises a first nucleic acid sequence of SEQ ID NO: 25, and the antisense strand comprises a second nucleic acid sequence of SEQ ID NO: 26;
(n) the sense strand comprises a first nucleic acid sequence of SEQ ID NO: 27, and the antisense strand comprises a second nucleic acid sequence of SEQ ID NO: 28; and
(o) the sense strand comprises a first nucleic acid sequence of SEQ ID NO: 29, and the antisense strand comprises a second nucleic acid sequence of SEQ ID NO: 30,
   wherein optionally one or more nucleotides of the sense strand and the antisense strand are independently modified nucleotides, and wherein optionally one or more internucleotide linkages of the sense strand and the antisense strand are modified internucleotide linkages.

The ATXN2 RNAi agents described herein may include modifications. The modifications can be made to one or more nucleotides of the sense strand and/or antisense strand or to the internucleotide linkages, which are the bonds between two nucleotides in the sense or antisense strand. For example, some 2′-modifications of ribose or deoxyribose can increase RNA or DNA stability and half-life. Such 2′-modifications can be 2′-fluoro, 2′-O-methyl (i.e., 2′-methoxy), 2′-O-alkyl, or 2′-O-methoxyethyl (2′-O-MOE).

In some embodiments, one or more nucleotides of the sense strand and/or the antisense strand are independently modified nucleotides, which means the sense strand and the antisense strand can have different modified nucleotides. In some embodiments, one or more nucleotides of the sense strand are modified nucleotides. In some embodiments, each nucleotide of the sense strand is a modified nucleotide. In some embodiments, one or more nucleotides of the antisense strand are modified nucleotides. In some embodiments, each nucleotide of the antisense strand is a modified nucleotide. In some embodiments, the modified nucleotide is a 2′-fluoro modified nucleotide, 2′-O-methyl modified nucleotide, or 2′-O-alkyl modified nucleotide. In some embodiments, each nucleotide of the sense strand and the antisense strand is independently a modified nucleotide, e.g., a 2′-fluoro modified nucleotide, 2′-O-methyl modified nucleotide, or 2′-O-alkyl modified nucleotide.

In some embodiments, the sense strand has four 2′-fluoro modified nucleotides, e.g., at positions 7, 9, 10, and 11 from the 5′ end of the sense strand. In some embodiments, nucleotides at positions other than positions 7, 9, 10, and 11 of the sense strand are 2′-O-methyl modified nucleotides. In some embodiments, the antisense strand has four 2′-fluoro modified nucleotides, e.g., at positions 2, 6, 14, and 16 from the 5′ end of the antisense strand. In some embodiments, nucleotides at positions other than positions 2, 6, 14 and 16 of the antisense strand are 2′-O-methyl modified nucleotides.

In some embodiments, the modified nucleotide is a 2′-O-alkyl modified nucleotide, which can serve as a delivery moiety. In some embodiments, the 2′-O-alkyl modified nucleotide is a 2′-O-hexadecyl uridine, 2′-O-hexadecyl cytidine, 2′-O-hexadecyl guanine, or 2′-O-hexadecyl adenosine. In some embodiments, 2′-O-hexadecyl uridine, 2′-O-hexadecyl cytidine, 2′-O-hexadecyl guanine, or 2′-O-hexadecyl adenosine is a modified nucleotide in the sense strand.

In some embodiments, the first nucleotide from the 5′ end of the antisense strand is a modified nucleotide that has a phosphate analog, e.g., 5′-vinylphosphonate (5′-VP).

In some embodiments, the sense strand has an abasic moiety or inverted abasic moiety, e.g., a moiety shown in Table 3, e.g., at position 9, 10 or 11.

In some embodiments, the sense strand and the antisense strand have one or more modified internucleotide linkages. In some embodiments, the modified internucleotide linkage is phosphorothioate linkage. In some embodiments, the sense strand has four or five phosphorothioate linkages. In some embodiments, the antisense strand has four or five phosphorothioate linkages. In some embodiments, the sense strand and the antisense strand each has four or five phosphorothioate linkages. In some embodiments, the sense strand has four phosphorothioate linkages and the antisense strand has four phosphorothioate linkages.

In a further aspect, provided herein are ATXN2 RNAi agent comprising a sense strand and an antisense strand, wherein the sense strand and the antisense strand form a duplex, wherein the sense strand and the antisense strand comprise a pair of nucleic acid sequences selected from the group consisting of:
(a) the sense strand comprises a first nucleic acid sequence of SEQ ID NO: 31, and the antisense strand comprises a second nucleic acid sequence of SEQ ID NO: 32;
(b) the sense strand comprises a first nucleic acid sequence of SEQ ID NO: 33, and the antisense strand comprises a second nucleic acid sequence of SEQ ID NO: 34;
(c) the sense strand comprises a first nucleic acid sequence of SEQ ID NO: 35, and the antisense strand comprises a second nucleic acid sequence of SEQ ID NO: 36;
(d) the sense strand comprises a first nucleic acid sequence of SEQ ID NO: 37, and the antisense strand comprises a second nucleic acid sequence of SEQ ID NO: 38;
(e) the sense strand comprises a first nucleic acid sequence of SEQ ID NO: 39, and the antisense strand comprises a second nucleic acid sequence of SEQ ID NO: 40;
(f) the sense strand comprises a first nucleic acid sequence of SEQ ID NO: 41, and the antisense strand comprises a second nucleic acid sequence of SEQ ID NO: 42;
(g) the sense strand comprises a first nucleic acid sequence of SEQ ID NO: 43, and the antisense strand comprises a second nucleic acid sequence of SEQ ID NO: 44;
(h) the sense strand comprises a first nucleic acid sequence of SEQ ID NO: 45, and the antisense strand comprises a second nucleic acid sequence of SEQ ID NO: 46;
(i) the sense strand comprises a first nucleic acid sequence of SEQ ID NO: 47, and the antisense strand comprises a second nucleic acid sequence of SEQ ID NO: 48;
(j) the sense strand comprises a first nucleic acid sequence of SEQ ID NO: 49, and the antisense strand comprises a second nucleic acid sequence of SEQ ID NO: 50;
(k) the sense strand comprises a first nucleic acid sequence of SEQ ID NO: 51, and the antisense strand comprises a second nucleic acid sequence of SEQ ID NO: 52;
(l) the sense strand comprises a first nucleic acid sequence of SEQ ID NO: 53, and the antisense strand comprises a second nucleic acid sequence of SEQ ID NO: 54;
(m) the sense strand comprises a first nucleic acid sequence of SEQ ID NO: 55, and the antisense strand comprises a second nucleic acid sequence of SEQ ID NO: 56;
(n) the sense strand comprises a first nucleic acid sequence of SEQ ID NO: 57, and the antisense strand comprises a second nucleic acid sequence of SEQ ID NO: 58; and
(o) the sense strand comprises a first nucleic acid sequence of SEQ ID NO: 59, and the antisense strand comprises a second nucleic acid sequence of SEQ ID NO: 60.

In some embodiments, provided herein are ATXN2 RNAi agent comprising a sense strand and an antisense strand, wherein the sense strand and the antisense strand form a duplex, wherein the sense strand and the antisense strand have a pair of nucleic acid sequences selected from the group consisting of:
(a) the sense strand consists of a first nucleic acid sequence of SEQ ID NO: 31, and the antisense strand consists of a second nucleic acid sequence of SEQ ID NO: 32;
(b) the sense strand consists of a first nucleic acid sequence of SEQ ID NO: 33, and the antisense strand consists of a second nucleic acid sequence of SEQ ID NO: 34;
(c) the sense strand consists of a first nucleic acid sequence of SEQ ID NO: 35, and the antisense strand consists of a second nucleic acid sequence of SEQ ID NO: 36;
(d) the sense strand consists of a first nucleic acid sequence of SEQ ID NO: 37, and the antisense strand consists of a second nucleic acid sequence of SEQ ID NO: 38;
(e) the sense strand consists of a first nucleic acid sequence of SEQ ID NO: 39, and the antisense strand consists of a second nucleic acid sequence of SEQ ID NO: 40;
(f) the sense strand consists of a first nucleic acid sequence of SEQ ID NO: 41, and the antisense strand consists of a second nucleic acid sequence of SEQ ID NO: 42;
(g) the sense strand consists of a first nucleic acid sequence of SEQ ID NO: 43, and the antisense strand consists of a second nucleic acid sequence of SEQ ID NO: 44;
(h) the sense strand consists of a first nucleic acid sequence of SEQ ID NO: 45, and the antisense strand consists of a second nucleic acid sequence of SEQ ID NO: 46;
(i) the sense strand consists of a first nucleic acid sequence of SEQ ID NO: 47, and the antisense strand consists of a second nucleic acid sequence of SEQ ID NO: 48;
(j) the sense strand consists of a first nucleic acid sequence of SEQ ID NO: 49, and the antisense strand consists of a second nucleic acid sequence of SEQ ID NO: 50;
(k) the sense strand consists of a first nucleic acid sequence of SEQ ID NO: 51, and the antisense strand consists of a second nucleic acid sequence of SEQ ID NO: 52;
(l) the sense strand consists of a first nucleic acid sequence of SEQ ID NO: 53, and the antisense strand consists of a second nucleic acid sequence of SEQ ID NO: 54;
(m) the sense strand consists of a first nucleic acid sequence of SEQ ID NO: 55, and the antisense strand consists of a second nucleic acid sequence of SEQ ID NO: 56;
(n) the sense strand consists of a first nucleic acid sequence of SEQ ID NO: 57, and the antisense strand consists of a second nucleic acid sequence of SEQ ID NO: 58; and
(o) the sense strand consists of a first nucleic acid sequence of SEQ ID NO: 59, and the antisense strand consists of a second nucleic acid sequence of SEQ ID NO: 60.

TABLE 2
ATXN2 RNAi Agents with Modifications

| ATXN2 | | | |
|---|---|---|---|
| RNAi | | | SEQ |
| Agent | | Sequence from | ID |
| No. | Strand | 5′ to 3′ end | NO |
| 16 | S | mC*mG*mAmAmAmCfGmUfGfAfAmGmAmAmA | 31 |
| | | mUmAmAmU*mG*mA | |
| | AS | PmU*fC*mAmUmUfAmUmUmUmCmUmUmCfAm | 32 |
| | | CfGmUmUmUmCmG*mG*mC | |
| 17 | S | mC*mC*mUmAmAmAfCmGfCfAfUmGmUmCmUm | 33 |
| | | UmCmAmG*mA*mA | |
| | AS | PmU*fU*mCmUmGfAmAmGmAmCmAmUmGfCm | 34 |
| | | GfUmUmUmAmGmG*mC*mA | |
| 18 | S | mG*mA*mGmAmUmGfCmUfUfUfUmAmCmUmG | 35 |
| | | mAmCmUmC*mU*mA | |
| | AS | PmU*fA*mGmAmGfUmCmAmGmUmAmAmAfAm | 36 |
| | | GfCmAmUmCmUmC*mU*mU | |
| 19 | S | mA*mA*mAmCmGmUfGmAfAfGfAmAmAmUmA | 37 |
| | | mAmUmGmG*mA*mA | |
| | AS | PmU*fU*mCmCmAfUmUmAmUmUmUmCmUfUm | 38 |
| | | CfAmCmGmUmUmU*mC*mG | |
| 20 | S | mG*mC*mUmCmCmUfGmUfCfUfCmUmAmCmUm | 39 |
| | | AmUmGmC*mC*mA | |
| | AS | PmU*fG*mGmCmAfUmAmGmUmAmGmAmGfAm | 40 |
| | | CfAmGmGmAmGmC*mU*mG | |
| 21 | S | mU*mG*mGmUmUmCfUmCfCfAfGmCmUmCmCm | 41 |
| | | UmGmUmC*mU*mA | |
| | AS | PmU*fA*mGmAmCfAmGmGmAmGmCmUmGfGm | 42 |
| | | AfGmAmAmCmCmA*mU*mG | |
| 22 | S | mG*mC*mCmUmAmAfAmCfGfCfAmUmGmUmCm | 43 |
| | | UmUmCmA*mG*mA | |
| | AS | PmU*fC*mUmGmAfAmGmAmCmAmUmGmCfGm | 44 |
| | | UfUmUmAmGmGmC*mA*mU | |
| 23 | S | mU*mU*mAmUmCmAfGmUfCfUfCmAmGmCmAm | 45 |
| | | UmCmCmU*mC*mA | |
| | AS | PmU*fG*mAmGmGfAmUmGmCmUmGmAmGfAm | 46 |
| | | CfUmGmAmUmAmA*mU*mG | |
| 24 | S | mU*mC*mUmCmCmAfGmCfUfCfCmUmGmUmCm | 47 |
| | | UmCmUmA*mC*mA | |
| | AS | PmU*fG*mUmAmGfAmGmAmCmAmGmGmAfGm | 48 |
| | | CfUmGmGmAmGmA*mA*mC | |
| 25 | S | mG*mC*mUmCmAmUfGmGfUfUfCmUmCmCmAm | 49 |
| | | GmCmUmC*mC*mA | |
| | AS | PmU*fG*mGmAmGfCmUmGmGmAmGmAmAfCm | 50 |
| | | CfAmUmGmAmGmC*mA*mG | |
| 26 | S | mC*mA*mGmUmCmAfGmGfUfCfCmCmAmAmCm | 51 |
| | | UmCmUmC*mU*mA | |
| | AS | PmU*fA*mGmAmGfAmGmUmUmGmGmGmAfCm | 52 |
| | | CfUmGmAmCmUmG*mG*mU | |
| 27 | S | mG*mA*mGmUmAmGfUmUfAfAfUmGmGmAmG | 53 |
| | | mGmUmGmU*mU*mA | |
| | AS | PmU*fA*mAmCmAfCmCmUmCmCmAmUmUfAm | 54 |
| | | AfCmUmAmCmUmC*mU*mU | |
| 28 | S | mA*mG*mUmCmAmGfGmUfCfCfCmAmAmCmUm | 55 |
| | | CmUmCmU*mU*mA | |
| | AS | PmU*fA*mAmGmAfGmAmGmUmUmGmGmGfAm | 56 |
| | | CfCmUmGmAmCmU*mG*mG | |
| 29 | S | mC*mC*mCmAmCmCfUmUfCfUfCmGmCmUmAm | 57 |
| | | CmCmAmG*mU*mA | |
| | AS | PmU*fA*mCmUmGfGmUmAmGmCmGmAmGfAm | 58 |
| | | AfGmGmUmGmGmG*mC*mG | |
| 30 | S | mC*mA*mAmGmAmCfAmUfAfUfAmGmAmGmCm | 59 |
| | | AmGmUmA*mC*mA | |
| | AS | PmU*fG*mUmAmCfUmGmCmUmCmUmAmUfAm | 60 |
| | | UfGmUmCmUmUmG*mG*mC | |

Abbreviations - “m” indicates 2′-OMe; “f” indicates 2′-fluoro; “*” indicates phosphorothioate linkage; “P” indicates 5′-phosphate; “S” means the sense strand; “AS” means the antisense strand.

TABLE 3
Abasic and inverted abasic (iAb) moieties

| Structure | |
|---|---|
| 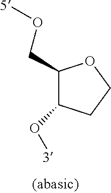 | 1 |
| 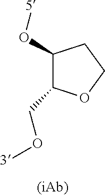 | 2 |

In some embodiments, the sense strand of the ATXN2 RNAi agent has a delivery moiety conjugated to the 3′ end of the sense strand. In some embodiments, the sense strand of the ATXN2 RNAi agent has a delivery moiety conjugated to a nucleotide of the sense strand. The delivery moiety can facilitate the entry of RNAi agent into the cells. In some embodiments, the delivery moiety is α-tocopherol, cholesterol or palmitic acid (see Table 4). In some embodiments, the delivery moiety is a known delivery moiety for delivering RNAi agent into a cell. Placement of a delivery moiety on the RNAi agent needs to overcome potential inefficient loading of AGO2 (Argonaute-2), or other hindrance of the RNA-induced silencing complex (RISC) complex activity.

In some embodiments, the delivery moiety is conjugated to the 3′ end of the sense stand via a linker. In some embodiments, the linker is selected from Linker 1, Linker 2, Linker 3, or Linker 4 of Table 5. Other suitable linkers are known in the art. Exemplary linker-delivery moiety pairs are shown in Table 6. In some embodiments, the ATXN2 RNAi agent has a linker-delivery moiety pair of Table 6.

In some embodiments, the delivery moiety is conjugated to a nucleotide of the sense strand. In that case, the delivery moiety is a modified nucleotide located in the sense strand. In some embodiments, the modified nucleotide is 2′-O-hexadecyl uridine, 2′-O-hexadecyl cytidine, 2′-O-hexadecyl guanine, or 2′-O-hexadecyl adenosine (Table 4).

TABLE 4
Delivery Moieties

| Delivery Moiety | Structure |
|---|---|
| 1 | α-Tocopherol |
| | 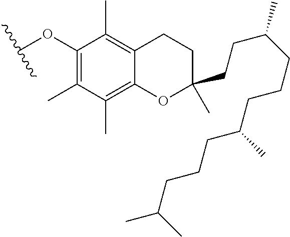 [SMILES: *Oc1c(C)c(C)c2c(c1C)CC[C@@](C)(CC[C@H](C)CCC[C@H](C)CCCC(C)C)O2] |
| 2 | Palmitic Acid |
| | 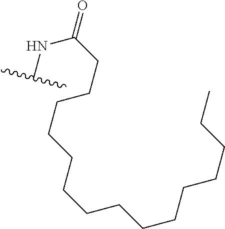 [SMILES: *NC(=O)CCCCCCCCCCCCCCC] |
| 3 | Uhd (2′-O-hexadecyl uridine) |
| | 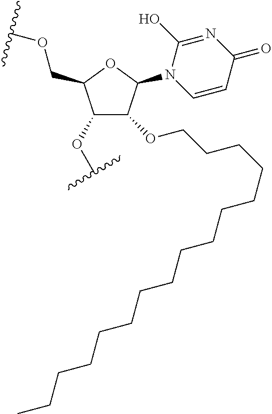 [SMILES: *OC[C@H]1O[C@@H](n2ccc(=O)nc2O)[C@H](OCCCCCCCCCCCCCCCC)[C@@H]1O*] |
| 4 | Ahd (2′-O-hexadecyl adenosine) |
| | 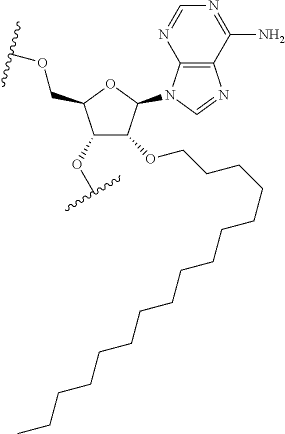 [SMILES: *OC[C@H]1O[C@@H](n2cnc3c(N)ncnc32)[C@H](OCCCCCCCCCCCCCCCC)[C@@H]1O*] |
| 5 | Chd (2′-O-hexadecyl cytidine) |
| | 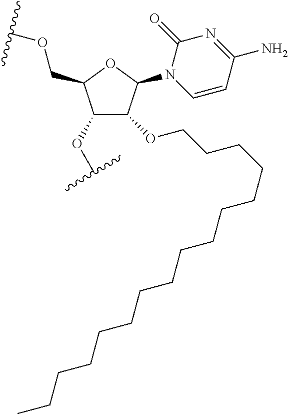 [SMILES: *OC[C@H]1O[C@@H](n2ccc(N)nc2=O)[C@H](OCCCCCCCCCCCCCCCC)[C@@H]1O*] |
| 6 | Ghd (2′-O-hexadecyl guanine) |
| | 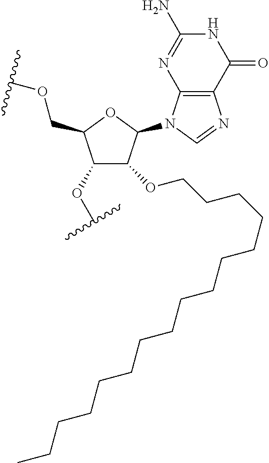 [SMILES: *OC[C@H]1O[C@@H](n2cnc3c(=O)[nH]c(N)nc32)[C@H](OCCCCCCCCCCCCCCCC)[C@@H]1O*] |
| 7 | Cholesterol |
| | 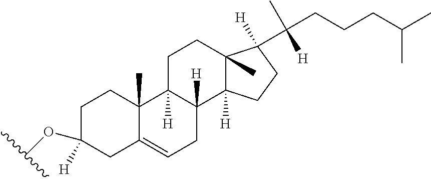 |

TABLE 5
Linkers

| Linker | Structure |
|---|---|
| 1 | Teg (tetraethylene glycol) linker |
| | 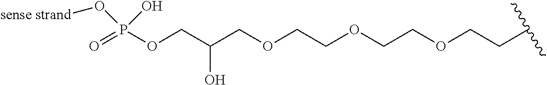 |
| 2 | Piperidinol-PEG linker |
| | 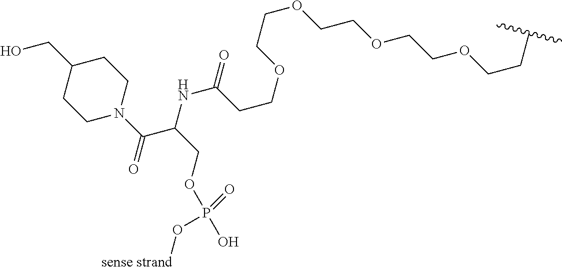 |
| 3 | Teg (tetraethylene glycol) linker PS |
| | 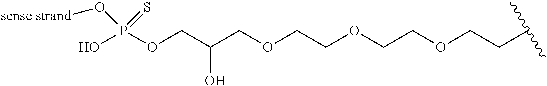 |
| 4 | Piperidinol-PEG linker PS |
| | 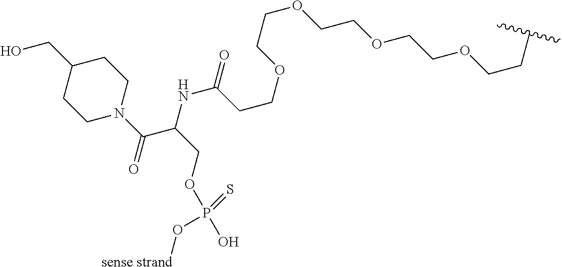 |

TABLE 6
Linker Delivery Moiety Pairs (LDP)

| LDP | Linker | Delivery Moiety |
|---|---|---|
| 1 | Teg (tetraethylene glycol) linker | α-Tocopherol |
| | 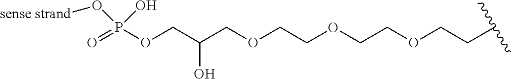 | 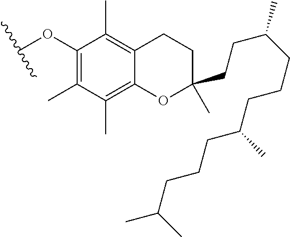 [SMILES: *Oc1c(C)c(C)c2c(c1C)CC[C@@](C)(CC[C@H](C)CCC[C@H](C)CCCC(C)C)O2] |
| 2 | Piperidinol-PEG linker | α-Tocopherol |
| | 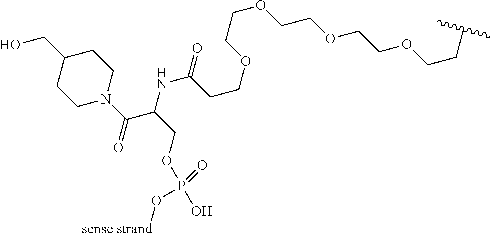 | 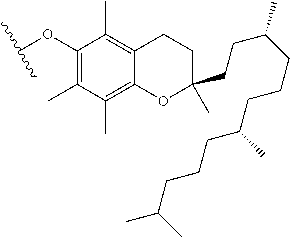 [SMILES: *Oc1c(C)c(C)c2c(c1C)CC[C@@](C)(CC[C@H](C)CCC[C@H](C)CCCC(C)C)O2] |
| 3 | Piperidinol-PEG linker | Palmitic Acid |
| | 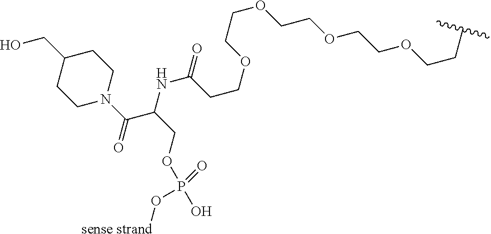 | 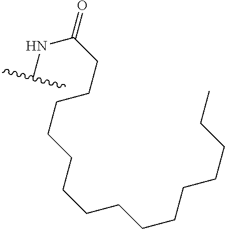 [SMILES: *NC(=O)CCCCCCCCCCCCCCC] |
| 4 | None | Uhd |
| | | 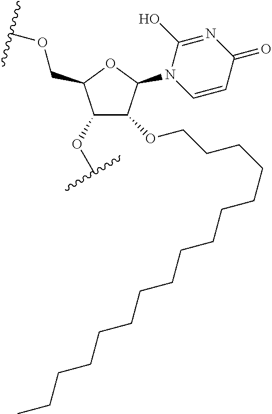 [SMILES: *OC[C@H]1O[C@@H](n2ccc(=O)nc2O)[C@H](OCCCCCCCCCCCCCCCC)[C@@H]1O*] |
| 5 | None | Ahd |
| | | 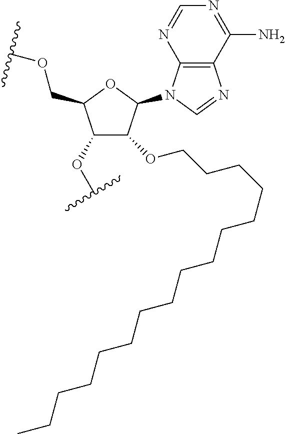 [SMILES: *OC[C@H]1O[C@@H](n2cnc3c(N)ncnc32)[C@H](OCCCCCCCCCCCCCCCC)[C@@H]1O*] |
| 6 | None | Chd |
| | | 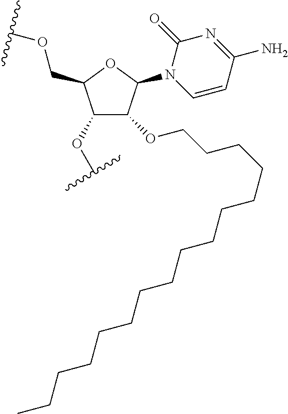 [SMILES: *OC[C@H]1O[C@@H](n2ccc(N)nc2=O)[C@H](OCCCCCCCCCCCCCCCC)[C@@H]1O*] |
| 7 | None | Ghd |
| | | 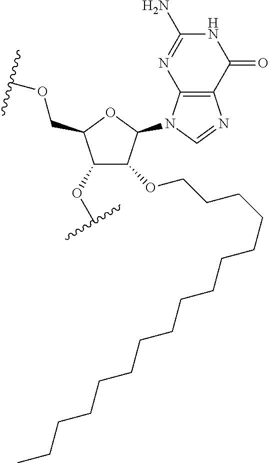 [SMILES: *OC[C@H]1O[C@@H](n2cnc3c(=O)[nH]c(N)nc32)[C@H](OCCCCCCCCCCCCCCCC)[C@@H]1O*] |
| 8 | Teg (tetraethylene glycol) linker | Cholesterol |
| | 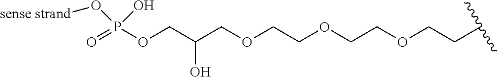 | 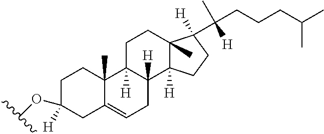 |

In a further aspect, provided herein are ATXN2 RNAi agents of Formula (I): R-L-D, wherein R is a double stranded RNA (dsRNA) comprising a sense stand and an antisense strand, wherein the sense strand and the antisense strand form a duplex; wherein D is a delivery means for delivering the dsRNA into a cell; and wherein L is a linking means for linking the dsRNA to the delivery means, or optionally absent, wherein the sense strand and the antisense strand comprise a pair of nucleic acid sequences selected from the group consisting of:
(a) the sense strand comprises a first nucleic acid sequence of SEQ ID NO: 1, and the antisense strand comprises a second nucleic acid sequence of SEQ ID NO: 2;
(b) the sense strand comprises a first nucleic acid sequence of SEQ ID NO: 3, and the antisense strand comprises a second nucleic acid sequence of SEQ ID NO: 4;
(c) the sense strand comprises a first nucleic acid sequence of SEQ ID NO: 5, and the antisense strand comprises a second nucleic acid sequence of SEQ ID NO: 6;
(d) the sense strand comprises a first nucleic acid sequence of SEQ ID NO: 7, and the antisense strand comprises a second nucleic acid sequence of SEQ ID NO: 8;
(e) the sense strand comprises a first nucleic acid sequence of SEQ ID NO: 9, and the antisense strand comprises a second nucleic acid sequence of SEQ ID NO: 10;
(f) the sense strand comprises a first nucleic acid sequence of SEQ ID NO: 11, and the antisense strand comprises a second nucleic acid sequence of SEQ ID NO: 12;
(g) the sense strand comprises a first nucleic acid sequence of SEQ ID NO: 13, and the antisense strand comprises a second nucleic acid sequence of SEQ ID NO: 14;
(h) the sense strand comprises a first nucleic acid sequence of SEQ ID NO: 15, and the antisense strand comprises a second nucleic acid sequence of SEQ ID NO: 16;
(i) the sense strand comprises a first nucleic acid sequence of SEQ ID NO: 17, and the antisense strand comprises a second nucleic acid sequence of SEQ ID NO: 18;
(j) the sense strand comprises a first nucleic acid sequence of SEQ ID NO: 19, and the antisense strand comprises a second nucleic acid sequence of SEQ ID NO: 20;
(k) the sense strand comprises a first nucleic acid sequence of SEQ ID NO: 21, and the antisense strand comprises a second nucleic acid sequence of SEQ ID NO: 22;
(l) the sense strand comprises a first nucleic acid sequence of SEQ ID NO: 23, and the antisense strand comprises a second nucleic acid sequence of SEQ ID NO: 24;
(m) the sense strand comprises a first nucleic acid sequence of SEQ ID NO: 25, and the antisense strand comprises a second nucleic acid sequence of SEQ ID NO: 26;
(n) the sense strand comprises a first nucleic acid sequence of SEQ ID NO: 27, and the antisense strand comprises a second nucleic acid sequence of SEQ ID NO: 28; and
(o) the sense strand comprises a first nucleic acid sequence of SEQ ID NO: 29, and the antisense strand comprises a second nucleic acid sequence of SEQ ID NO: 30,
   wherein optionally one or more nucleotides of the sense strand and the antisense strand are independently modified nucleotides, and wherein optionally one or more internucleotide linkages of the sense strand and the antisense strand are modified internucleotide linkages.

In some embodiments, provided herein are ATXN2 RNAi agents of Formula (I): R-L-D, wherein R is a dsRNA comprising a sense stand and an antisense strand, wherein the sense strand and the antisense strand form a duplex; wherein D is a delivery moiety; and wherein L is a linker or optionally absent, wherein the sense strand and the antisense strand comprise a pair of nucleic acid sequences selected from the group consisting of:
(a) the sense strand comprises a first nucleic acid sequence of SEQ ID NO: 1, and the antisense strand comprises a second nucleic acid sequence of SEQ ID NO: 2;
(b) the sense strand comprises a first nucleic acid sequence of SEQ ID NO: 3, and the antisense strand comprises a second nucleic acid sequence of SEQ ID NO: 4;
(c) the sense strand comprises a first nucleic acid sequence of SEQ ID NO: 5, and the antisense strand comprises a second nucleic acid sequence of SEQ ID NO: 6;
(d) the sense strand comprises a first nucleic acid sequence of SEQ ID NO: 7, and the antisense strand comprises a second nucleic acid sequence of SEQ ID NO: 8;
(e) the sense strand comprises a first nucleic acid sequence of SEQ ID NO: 9, and the antisense strand comprises a second nucleic acid sequence of SEQ ID NO: 10;
(f) the sense strand comprises a first nucleic acid sequence of SEQ ID NO: 11, and the antisense strand comprises a second nucleic acid sequence of SEQ ID NO: 12;
(g) the sense strand comprises a first nucleic acid sequence of SEQ ID NO: 13, and the antisense strand comprises a second nucleic acid sequence of SEQ ID NO: 14;
(h) the sense strand comprises a first nucleic acid sequence of SEQ ID NO: 15, and the antisense strand comprises a second nucleic acid sequence of SEQ ID NO: 16;
(i) the sense strand comprises a first nucleic acid sequence of SEQ ID NO: 17, and the antisense strand comprises a second nucleic acid sequence of SEQ ID NO: 18;
(j) the sense strand comprises a first nucleic acid sequence of SEQ ID NO: 19, and the antisense strand comprises a second nucleic acid sequence of SEQ ID NO: 20;
(k) the sense strand comprises a first nucleic acid sequence of SEQ ID NO: 21, and the antisense strand comprises a second nucleic acid sequence of SEQ ID NO: 22;
(l) the sense strand comprises a first nucleic acid sequence of SEQ ID NO: 23, and the antisense strand comprises a second nucleic acid sequence of SEQ ID NO: 24;
(m) the sense strand comprises a first nucleic acid sequence of SEQ ID NO: 25, and the antisense strand comprises a second nucleic acid sequence of SEQ ID NO: 26;
(n) the sense strand comprises a first nucleic acid sequence of SEQ ID NO: 27, and the antisense strand comprises a second nucleic acid sequence of SEQ ID NO: 28; and
(o) the sense strand comprises a first nucleic acid sequence of SEQ ID NO: 29, and the antisense strand comprises a second nucleic acid sequence of SEQ ID NO: 30,
   wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand and the antisense strand in RNAi agents of Formula (I) comprise a pair of nucleic acid sequences selected from the group consisting of:
(a) the sense strand comprises a first nucleic acid sequence of SEQ ID NO: 31, and the antisense strand comprises a second nucleic acid sequence of SEQ ID NO: 32;
(b) the sense strand comprises a first nucleic acid sequence of SEQ ID NO: 33, and the antisense strand comprises a second nucleic acid sequence of SEQ ID NO: 34;
(c) the sense strand comprises a first nucleic acid sequence of SEQ ID NO: 35, and the antisense strand comprises a second nucleic acid sequence of SEQ ID NO: 36;
(d) the sense strand comprises a first nucleic acid sequence of SEQ ID NO: 37, and the antisense strand comprises a second nucleic acid sequence of SEQ ID NO: 38;
(e) the sense strand comprises a first nucleic acid sequence of SEQ ID NO: 39, and the antisense strand comprises a second nucleic acid sequence of SEQ ID NO: 40;
(f) the sense strand comprises a first nucleic acid sequence of SEQ ID NO: 41, and the antisense strand comprises a second nucleic acid sequence of SEQ ID NO: 42;
(g) the sense strand comprises a first nucleic acid sequence of SEQ ID NO: 43, and the antisense strand comprises a second nucleic acid sequence of SEQ ID NO: 44;
(h) the sense strand comprises a first nucleic acid sequence of SEQ ID NO: 45, and the antisense strand comprises a second nucleic acid sequence of SEQ ID NO: 46;
(i) the sense strand comprises a first nucleic acid sequence of SEQ ID NO: 47, and the antisense strand comprises a second nucleic acid sequence of SEQ ID NO: 48;
(j) the sense strand comprises a first nucleic acid sequence of SEQ ID NO: 49, and the antisense strand comprises a second nucleic acid sequence of SEQ ID NO: 50;
(k) the sense strand comprises a first nucleic acid sequence of SEQ ID NO: 51, and the antisense strand comprises a second nucleic acid sequence of SEQ ID NO: 52;
(l) the sense strand comprises a first nucleic acid sequence of SEQ ID NO: 53, and the antisense strand comprises a second nucleic acid sequence of SEQ ID NO: 54;
(m) the sense strand comprises a first nucleic acid sequence of SEQ ID NO: 55, and the antisense strand comprises a second nucleic acid sequence of SEQ ID NO: 56;
(n) the sense strand comprises a first nucleic acid sequence of SEQ ID NO: 57, and the antisense strand comprises a second nucleic acid sequence of SEQ ID NO: 58;
(o) the sense strand comprises a first nucleic acid sequence of SEQ ID NO: 59, and the antisense strand comprises a second nucleic acid sequence of SEQ ID NO: 60.

In some embodiments, the sense strand and the antisense strand in RNAi agents of Formula (I) have a pair of nucleic acid sequences selected from the group consisting of:
(a) the sense strand consists of a first nucleic acid sequence of SEQ ID NO: 31, and the antisense strand consists of a second nucleic acid sequence of SEQ ID NO: 32;
(b) the sense strand consists of a first nucleic acid sequence of SEQ ID NO: 33, and the antisense strand consists of a second nucleic acid sequence of SEQ ID NO: 34;
(c) the sense strand consists of a first nucleic acid sequence of SEQ ID NO: 35, and the antisense strand consists of a second nucleic acid sequence of SEQ ID NO: 36;
(d) the sense strand consists of a first nucleic acid sequence of SEQ ID NO: 37, and the antisense strand consists of a second nucleic acid sequence of SEQ ID NO: 38;
(e) the sense strand consists of a first nucleic acid sequence of SEQ ID NO: 39, and the antisense strand consists of a second nucleic acid sequence of SEQ ID NO: 40;
(f) the sense strand consists of a first nucleic acid sequence of SEQ ID NO: 41, and the antisense strand consists of a second nucleic acid sequence of SEQ ID NO: 42;
(g) the sense strand consists of a first nucleic acid sequence of SEQ ID NO: 43, and the antisense strand consists of a second nucleic acid sequence of SEQ ID NO: 44;
(h) the sense strand consists of a first nucleic acid sequence of SEQ ID NO: 45, and the antisense strand consists of a second nucleic acid sequence of SEQ ID NO: 46;
(i) the sense strand consists of a first nucleic acid sequence of SEQ ID NO: 47, and the antisense strand consists of a second nucleic acid sequence of SEQ ID NO: 48;
(j) the sense strand consists of a first nucleic acid sequence of SEQ ID NO: 49, and the antisense strand consists of a second nucleic acid sequence of SEQ ID NO: 50;
(k) the sense strand consists of a first nucleic acid sequence of SEQ ID NO: 51, and the antisense strand consists of a second nucleic acid sequence of SEQ ID NO: 52;
(l) the sense strand consists of a first nucleic acid sequence of SEQ ID NO: 53, and the antisense strand consists of a second nucleic acid sequence of SEQ ID NO: 54;
(m) the sense strand consists of a first nucleic acid sequence of SEQ ID NO: 55, and the antisense strand consists of a second nucleic acid sequence of SEQ ID NO: 56;
(n) the sense strand consists of a first nucleic acid sequence of SEQ ID NO: 57, and the antisense strand consists of a second nucleic acid sequence of SEQ ID NO: 58; and
(o) the sense strand consists of a first nucleic acid sequence of SEQ ID NO: 59, and the antisense strand consists of a second nucleic acid sequence of SEQ ID NO: 60.

In some embodiments, the delivery means or delivery moiety is conjugated to the 3′ end of the sense strand. In some embodiments, the delivery means or delivery moiety is conjugated to a nucleotide of the sense strand. In some embodiments, the delivery means or delivery moiety is palmitic acid, cholesterol, or α-tocopherol. In some embodiments, the linking means or linker is selected from the group consisting of Linker 1, Linker 2, Linker 3, and Linker 4 of Table 5.

The sense strand and antisense strand of ATXN2 RNAi agent can be synthesized using any nucleic acid polymerization methods known in the art, for example, solid-phase synthesis by employing phosphoramidite chemistry methodology (e.g., Current Protocols in Nucleic Acid Chemistry, Beaucage, S. L. et al. (Edrs.), John Wiley & Sons, Inc., New York, NY, USA), H-phosphonate, phosphortriester chemistry, or enzymatic synthesis. Automated commercial synthesizers can be used, for example, MerMade™ 12 from LGC Biosearch Technologies, or other synthesizers from BioAutomation or Applied Biosystems. Phosphorothioate linkages can be introduced using a sulfurizing reagent such as phenylacetyl disulfide or DDTT (((dimethylaminomethylidene)amino)-3H-1,2,4-dithiazaoline-3-thione). It is well known to use similar techniques and commercially available modified amidites and controlled-pore glass (CPG) products to synthesize modified oligonucleotides or conjugated oligonucleotides.

Purification methods can be used to exclude the unwanted impurities from the final oligonucleotide product. Commonly used purification techniques for single stranded oligonucleotides include reverse-phase ion pair high performance liquid chromatography (RP-IP-HPLC), capillary gel electrophoresis (CGE), anion exchange HPLC (AX-HPLC), and size exclusion chromatography (SEC). After purification, oligonucleotides can be analyzed by mass spectrometry and quantified by spectrophotometry at a wavelength of 260 nm. The sense strand and antisense strand can then be annealed to form a duplex.

In another aspect, provided herein are pharmaceutical compositions comprising an ATXN2 RNAi agent described herein and a pharmaceutically acceptable carrier. Also provided herein are pharmaceutical compositions comprising a means for reducing ATXN2 expression in a cell and a pharmaceutically acceptable carrier. Such pharmaceutical compositions can also comprise one or more pharmaceutically acceptable excipient, diluent, or carrier. Pharmaceutical compositions can be prepared by methods well known in the art (e.g., Remington: The Science and Practice of Pharmacy, 23rd edition (2020), A. Loyd et al., Academic Press).

In a further aspect, provided herein are methods of reducing ATXN2 expression in a cell (e.g., a neuron), such methods can include introducing an ATXN2 RNAi agent described herein into the cell; and incubating the cell for a time sufficient for degradation of ATXN2 mRNA, thereby reducing ATXN2 expression in the cell. The ATXN2 RNAi agent can be introduced into the cell (e.g., a neuron) using a method known in the art, e.g., transfection, electroporation, microinjection, or uptake by the cell via natural transport mechanisms.

In another aspect, provided herein are methods of reducing ATXN2 expression in a patient in need thereof, and such method comprises administering to the patient an effective amount of an ATXN2 RNAi agent or a pharmaceutical composition described herein.

In another aspect, provided herein are methods of treating an ATXN2-associated neurological disease in a patient in need thereof, and such method comprises administering to the patient an effective amount of the ATXN2 RNAi agent or a pharmaceutical composition described herein. Abnormal CAG trinucleotide expansion in ATXN2 gene is associated with spinocerebellar ataxia type 2 (SCA2) and amyotrophic lateral sclerosis (ALS). In another aspect, Ataxin-2 is known to be required for the toxicity mediated by abnormal aggregation of TAR DNA binding protein (TDP-43), which is believed to be the driver in 95% of sporadic and familial amyotrophic lateral sclerosis cases. Exemplary ATXN2-associated neurological diseases, includes, but are not limited to, spinocerebellar ataxia type 2 (SCA2), amyotrophic lateral sclerosis (ALS), primary lateral sclerosis (PLS), Parkinson's disease, Alzheimer's disease, frontotemporal lobar degeneration (FTLD), progressive muscular atrophy (PMA), multiple system proteinopathy, Perry disease, and TDP-43 proteinopathy, e.g., neurological disease associated with abnormal TDP-43 aggregation (de Boer et al, 2021, J Neurol. Neurosurg. Psychiatry. 2020 Nov. 11; 92 (1): 86-95).

The ATXN2 RNAi agent can be administered to the patient intrathecally, intracerebroventricularly, or via intracisternal magna injection. In some embodiments, the ATXN2 RNAi agent is administered to the patient intrathecally, e.g., via a catheter or direct injection into the intrathecal space.

RNAi dosage regimens may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In another aspect, provided herein are ATXN2 RNAi agents or pharmaceutical compositions comprising an ATXN2 RNAi agent for use in reducing ATXN2 expression. Also provided herein are ATXN2 RNAi agents or the pharmaceutical composition comprising an ATXN2 RNAi agent for use in a therapy. Also provided herein are ATXN2 RNAi agents or pharmaceutical compositions comprising an ATXN2 RNAi agent for use in the treatment of an ATXN2-associated neurological disease. Also provided herein are uses of ATXN2 RNAi agents in the manufacture of a medicament for the treatment of an ATXN2-associated neurological disease.

As used herein, the terms “a,” “an,” “the,” and similar terms used in the context of the present disclosure (especially in the context of the claims) are to be construed to cover both the singular and plural unless otherwise indicated herein or clearly contradicted by the context.

As used herein, the term “alkyl” means saturated linear or branched-chain monovalent hydrocarbon radical, containing the indicated number of carbon atoms. For example, “C₁-C₂₀ alkyl” means a radical having 1-20 carbon atoms in a linear or branched arrangement.

As used herein, “antisense strand” means a single-stranded oligonucleotide that is complementary to a region of a target sequence. Likewise, and as used herein, “sense strand” means a single-stranded oligonucleotide that is complementary to a region of an antisense strand.

As used herein, “complementary” means a structural relationship between two nucleotides (e.g., on two opposing nucleic acids or on opposing regions of a single nucleic acid strand, e.g., a hairpin) that permits the two nucleotides to form base pairs with one another. For example, a purine nucleotide of one nucleic acid that is complementary to a pyrimidine nucleotide of an opposing nucleic acid may base pair together by forming hydrogen bonds with one another. Complementary nucleotides can base pair in the Watson-Crick manner or in any other manner that allows for the formation of stable duplexes. Likewise, two nucleic acids may have regions of multiple nucleotides that are complementary with each other to form regions of complementarity, as described herein.

As used herein, a “delivery moiety” refers to a chemical moiety that facilitates the entry of an oligonucleotide or RNAi agent into a cell. The delivery moiety can be lipid, cholesterol, vitamin E, carbohydrate, amino sugar, or polypeptide. In some embodiments, the delivery moiety is α-tocopherol, cholesterol or palmitic acid.

As used herein, “duplex,” in reference to nucleic acids or oligonucleotides, means a structure formed through complementary base pairing of two antiparallel sequences of nucleotides (i.e., in opposite directions), whether formed by two separate nucleic acid strands or by a single, folded strand (e.g., via a hairpin).

An “effective amount” refers to an amount necessary (for periods of time and for the means of administration) to achieve the desired therapeutic result. An effective amount of a RNAi agent may vary according to factors such as the disease state, age, sex, and weight of the individual, and the ability of the RNAi agent to elicit a desired response in the individual. An effective amount is also one in which any toxic or detrimental effects of the RNAi agent are outweighed by the therapeutically beneficial effects.

The term “knockdown” or “expression knockdown” refers to reduced mRNA or protein expression of a gene after treatment of a reagent, e.g., a RNAi agent.

As used herein, “modified internucleotide linkage” means an internucleotide linkage having one or more chemical modifications when compared with a reference internucleotide linkage having a phosphodiester bond. A modified internucleotide linkage can be a non-naturally occurring linkage. In some embodiments, the modified internucleotide linkage is phosphorothioate linkage.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide. In some embodiments, the modified nucleotide is a 2′-fluoro modified nucleotide, 2′-O-methyl modified nucleotide, or 2′-O-alkyl modified nucleotide. In some embodiments, the modified nucleotide has a phosphate analog, e.g., 5′-vinylphosphonate. In some embodiments, the modified nucleotide has an abasic moiety or inverted abasic moiety, e.g., a moiety shown in Table 3, e.g., at position 9, 10 or 11.

As used herein, “nucleotide” means an organic compound having a nucleoside (a nucleobase, e.g., adenine, cytosine, guanine, thymine, or uracil, and a pentose sugar, e.g., ribose or 2′-deoxyribose) linked to a phosphate group. A “nucleotide” can serve as a monomeric unit of nucleic acid polymers such as deoxyribonucleic acid (DNA) and ribonucleic acid (RNA).

As used herein, “oligonucleotide” means a polymer of linked nucleotides, each of which can be modified or unmodified. An oligonucleotide is typically less than about 100 nucleotides in length.

As used herein, “overhang” means the unpaired nucleotide or nucleotides that protrude from the duplex structure of a double stranded oligonucleotide. An overhang may include one or more unpaired nucleotides extending from a duplex region at the 5′ terminus or 3′ terminus of a double stranded oligonucleotide. The overhang can be a 3′ or 5′ overhang on the antisense strand or sense strand of a double stranded oligonucleotide.

The term “patient”, as used herein, refers to a human patient.

As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. A 5′ phosphate analog can include a phosphatase-resistant linkage. Examples of phosphate analogs include 5′ methylene phosphonate (5′-MP) and 5′-(E)-vinylphosphonate (5′-VP). In some embodiments, the phosphate analog is 5′-VP.

The term “% sequence identity” or “percentage sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity. Alignment for purposes of determining percent nucleic acid sequence identity can be achieved in various ways that are within the skill in the art, for instance, using publicly available computer software programs, for example, those described in Current Protocols in Molecular Biology (Ausubel et al., eds., 1987, Supp. 30, section 7.7.18, Table 7.7.1), and including BLAST, BLAST-2, ALIGN, Megalign (DNASTAR), Clustal W2.0 or Clustal X2.0 software. Those skilled in the art can determine appropriate parameters for measuring alignment, including any algorithms needed to achieve maximal alignment over the full length of the sequences being compared. Percentage of “sequence identity” can be determined by comparing two optimally aligned sequences over a comparison window, where the fragment of the nucleic acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical nucleotide, nucleoside, or nucleobase occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

As used herein, “RNAi,” “RNAi agent,” “iRNA,” “iRNA agent,” and “RNA interference agent” means an agent that mediates sequence-specific degradation of a target mRNA by RNA interference, e.g., via RNA-induced silencing complex (RISC) pathway. In some embodiments, the RNAi agent has a sense strand and an antisense strand, and the sense strand and the antisense strand form a duplex (e.g., a double stranded RNA). In some embodiments, the sense strand has a delivery moiety conjugated to the 3′ end of the sense strand or a nucleotide of the sense strand.

As used herein, “strand” refers to a single, contiguous sequence of nucleotides linked together through internucleotide linkages (e.g., phosphodiester linkages or phosphorothioate linkages). A strand can have two free ends (e.g., a 5′ end and a 3′ end).

As used herein, “ATXN2” (also known as ATX2, SCA2, TNRC13) refers to a human ATXN2 mRNA transcript. The nucleotide sequence of human ATXN2 mRNA isoform 1 can be found at NM_002973.4:

| (SEQ ID NO: 61) | |
|---|---|
| 1 | AGAGCTCGCC TCCCTCCGCC TCAGACTGTT TTGGTAGCAA CGGCAACGGC GGCGGCGCGT |
| 61 | TTCGGCCCGG CTCCCGGCGG CTCCTTGGTC TCGGCGGGCC TCCCCGCCCC TTCGTCGTCC |
| 121 | TCCTTCTCCC CCTCGCCAGC CCGGGCGCCC CTCCGGCCGC GCCAACCCGC GCCTCCCCGC |
| 181 | TCGGCGCCCG CGCGTCCCCG CCGCGTTCCG GCGTCTCCTT GGCGCGCCCG GCTCCCGGCT |
| 241 | GTCCCCGCCC GGCGTGCGAG CCGGTGTATG GGCCCCTCAC CATGTCGCTG AAGCCCCAGC |
| 301 | AGCAGCAGCA GCAGCAGCAG CAGCAGCAGC AGCAGCAACA GCAGCAGCAG CAGCAGCAGC |
| 361 | AGCAGCCGCC GCCCGCGGCT GCCAATGTCC GCAAGCCCGG CGGCAGCGGC CTTCTAGCGT |
| 421 | CGCCCGCCGC CGCGCCTTCG CCGTCCTCGT CCTCGGTCTC CTCGTCCTCG GCCACGGCTC |
| 481 | CCTCCTCGGT GGTCGCGGCG ACCTCCGGCG GCGGGAGGCC CGGCCTGGGC AGAGGTCGAA |
| 541 | ACAGTAACAA AGGACTGCCT CAGTCTACGA TTTCTTTTGA TGGAATCTAT GCAAATATGA |
| 601 | GGATGGTTCA TATACTTACA TCAGTTGTTG GCTCCAAATG TGAAGTACAA GTGAAAAATG |
| 661 | GAGGTATATA TGAAGGAGTT TTTAAAACTT ACAGTCCGAA GTGTGATTTG GTACTTGATG |
| 721 | CCGCACATGA GAAAAGTACA GAATCCAGTT CGGGGCCGAA ACGTGAAGAA ATAATGGAGA |
| 781 | GTATTTTGTT CAAATGTTCA GACTTTGTTG TGGTACAGTT TAAAGATATG GACTCCAGTT |
| 841 | ATGCAAAAAG AGATGCTTTT ACTGACTCTG CTATCAGTGC TAAAGTGAAT GGCGAACACA |
| 901 | AAGAGAAGGA CCTGGAGCCC TGGGATGCAG GTGAACTCAC AGCCAATGAG GAACTTGAGG |
| 961 | CTTTGGAAAA TGACGTATCT AATGGATGGG ATCCCAATGA TATGTTTCGA TATAATGAAG |
| 1021 | AAAATTATGG TGTAGTGTCT ACGTATGATA GCAGTTTATC TTCGTATACA GTGCCCTTAG |
| 1081 | AAAGAGATAA CTCAGAAGAA TTTTTAAAAC GGGAAGCAAG GGCAAACCAG TTAGCAGAAG |
| 1141 | AAATTGAGTC AAGTGCCCAG TACAAAGCTC GAGTGGCCCT GGAAAATGAT GATAGGAGTG |
| 1201 | AGGAAGAAAA ATACACAGCA GTTCAGAGAA ATTCCAGTGA ACGTGAGGGG CACAGCATAA |
| 1261 | ACACTAGGGA AAATAAATAT ATTCCTCCTG GACAAAGAAA TAGAGAAGTC ATATCCTGGG |
| 1321 | GAAGTGGGAG ACAGAATTCA CCGCGTATGG GCCAGCCTGG ATCGGGCTCC ATGCCATCAA |
| 1381 | GATCCACTTC TCACACTTCA GATTTCAACC CGAATTCTGG TTCAGACCAA AGAGTAGTTA |
| 1441 | ATGGAGGTGT TCCCTGGCCA TCGCCTTGCC CATCTCCTTC CTCTCGCCCA CCTTCTCGCT |
| 1501 | ACCAGTCAGG TCCCAACTCT CTTCCACCTC GGGCAGCCAC CCCTACACGG CCGCCCTCCA |
| 1561 | GGCCCCCCTC GCGGCCATCC AGACCCCCGT CTCACCCCTC TGCTCATGGT TCTCCAGCTC |
| 1621 | CTGTCTCTAC TATGCCTAAA CGCATGTCTT CAGAAGGGCC TCCAAGGATG TCCCCAAAGG |
| 1681 | CCCAGCGACA TCCTCGAAAT CACAGAGTTT CTGCTGGGAG GGGTTCCATA TCCAGTGGCC |
| 1741 | TAGAATTTGT ATCCCACAAC CCACCCAGTG AAGCAGCTAC TCCTCCAGTA GCAAGGACCA |
| 1801 | GTCCCTCGGG GGGAACGTGG TCATCAGTGG TCAGTGGGGT TCCAAGATTA TCCCCTAAAA |
| 1861 | CTCATAGACC CAGGTCTCCC AGACAGAACA GTATTGGAAA TACCCCCAGT GGGCCAGTTC |
| 1921 | TTGCTTCTCC CCAAGCTGGT ATTATTCCAA CTGAAGCTGT TGCCATGCCT ATTCCAGCTG |
| 1981 | CATCTCCTAC GCCTGCTAGT CCTGCATCGA ACAGAGCTGT TACCCCTTCT AGTGAGGCTA |
| 2041 | AAGATTCCAG GCTTCAAGAT CAGAGGCAGA ACTCTCCTGC AGGGAATAAA GAAAATATTA |
| 2101 | AACCCAATGA AACATCACCT AGCTTCTCAA AAGCTGAAAA CAAAGGTATA TCACCAGTTG |
| 2161 | TTTCTGAACA TAGAAAACAG ATTGATGATT TAAAGAAATT TAAGAATGAT TTTAGGTTAC |
| 2221 | AGCCAAGTTC TACTTCTGAA TCTATGGATC AACTACTAAA CAAAAATAGA GAGGGAGAAA |
| 2281 | AATCAAGAGA TTTGATCAAA GACAAAATTG AACCAAGTGC TAAGGATTCT TTCATTGAAA |
| 2341 | ATAGCAGCAG CAACTGTACC AGTGGCAGCA GCAAGCCGAA TAGCCCCAGC ATTTCCCCTT |
| 2401 | CAATACTTAG TAACACGGAG CACAAGAGGG GACCTGAGGT CACTTCCCAA GGGGTTCAGA |
| 2461 | CTTCCAGCCC AGCATGTAAA CAAGAGAAAG ACGATAAGGA AGAGAAGAAA GACGCAGCTG |
| 2521 | AGCAAGTTAG GAAATCAACA TTGAATCCCA ATGCAAAGGA GTTCAACCCA CGTTCCTTCT |
| 2581 | CTCAGCCAAA GCCTTCTACT ACCCCAACTT CACCTCGGCC TCAAGCACAA CCTAGCCCAT |
| 2641 | CTATGGTGGG TCATCAACAG CCAACTCCAG TTTATACTCA GCCTGTTTGT TTTGCACCAA |
| 2701 | ATATGATGTA TCCAGTCCCA GTGAGCCCAG GCGTGCAACC TTTATACCCA ATACCTATGA |
| 2761 | CGCCCATGCC AGTGAATCAA GCCAAGACAT ATAGAGCAGT ACCAAATATG CCCCAACAGC |
| 2821 | GGCAAGACCA GCATCATCAG AGTGCCATGA TGCACCCAGC GTCAGCAGCG GGCCCACCGA |
| 2881 | TTGCAGCCAC CCCACCAGCT TACTCCACGC AATATGTTGC CTACAGTCCT CAGCAGTTCC |
| 2941 | CAAATCAGCC CCTTGTTCAG CATGTGCCAC ATTATCAGTC TCAGCATCCT CATGTCTATA |
| 3001 | GTCCTGTAAT ACAGGGTAAT GCTAGAATGA TGGCACCACC AACACACGCC CAGCCTGGTT |
| 3061 | TAGTATCTTC TTCAGCAACT CAGTACGGGG CTCATGAGCA GACGCATGCG ATGTATGCAT |
| 3121 | GTCCCAAATT ACCATACAAC AAGGAGACAA GCCCTTCTTT CTACTTTGCC ATTTCCACGG |
| 3181 | GCTCCCTTGC TCAGCAGTAT GCGCACCCTA ACGCTACCCT GCACCCACAT ACTCCACACC |
| 3241 | CTCAGCCTTC AGCTACCCCC ACTGGACAGC AGCAAAGCCA ACATGGTGGA AGTCATCCTG |
| 3301 | CACCCAGTCC TGTTCAGCAC CATCAGCACC AGGCCGCCCA GGCTCTCCAT CTGGCCAGTC |
| 3361 | CACAGCAGCA GTCAGCCATT TACCACGCGG GGCTTGCGCC AACTCCACCC TCCATGACAC |
| 3421 | CTGCCTCCAA CACGCAGTCG CCACAGAATA GTTTCCCAGC AGCACAACAG ACTGTCTTTA |
| 3481 | CGATCCATCC TTCTCACGTT CAGCCGGCGT ATACCAACCC ACCCCACATG GCCCACGTAC |
| 3541 | CTCAGGCTCA TGTACAGTCA GGAATGGTTC CTTCTCATCC AACTGCCCAT GCGCCAATGA |
| 3601 | TGCTAATGAC GACACAGCCA CCCGGCGGTC CCCAGGCCGC CCTCGCTCAA AGTGCACTAC |
| 3661 | AGCCCATTCC AGTCTCGACA ACAGCGCATT TCCCCTATAT GACGCACCCT TCAGTACAAG |
| 3721 | CCCACCACCA ACAGCAGTTG TAAGGCTGCC CTGGAGGAAC CGAAAGGCCA AATTCCCTCC |
| 3781 | TCCCTTCTAC TGCTTCTACC AACTGGAAGC ACAGAAAACT AGAATTTCAT TTATTTTGTT |
| 3841 | TTTAAAATAT ATATGTTGAT TTCTTGTAAC ATCCAATAGG AATGCTAACA GTTCACTTGC |
| 3901 | AGTGGAAGAT ACTTGGACCG AGTAGAGGCA TTTAGGAACT TGGGGGCTAT TCCATAATTC |
| 3961 | CATATGCTGT TTCAGAGTCC CGCAGGTACC CCAGCTCTGC TTGCCGAAAC TGGAAGTTAT |
| 4021 | TTATTTTTTA ATAACCCTTG AAAGTCATGA ACACATCAGC TAGCAAAAGA AGTAACAAGA |
| 4081 | GTGATTCTTG CTGCTATTAC TGCTAAAAAA AAAAAAAAAA AAAAATCAAG ACTTGGAACG |
| 4141 | CCCTTTTACT AAACTTGACA AAGTTTCAGT AAATTCTTAC CGTCAAACTG ACGGATTATT |
| 4201 | ATTTATAAAT CAAGTTTGAT GAGGTGATCA CTGTCTACAG TGGTTCAACT TTTAAGTTAA |
| 4261 | GGGAAAAACT TTTACTTTGT AGATAATATA AAATAAAAAC TTAAAAAAAA TTTAAAAAAT |
| 4321 | AAAAAAAGTT TTAAAAACTG A. |

The corresponding amino acid sequence of human ATXN2 protein isoform 1 can be found at NP_002964.4:

| (SEQ ID NO: 62) | |
|---|---|
| 1 | MSLKPQQQQQ QQQQQQQQQQ QQQQQQQQPP PAAANVRKPG GSGLLASPAA APSPSSSSVS |
| 61 | SSSATAPSSV VAATSGGGRP GLGRGRNSNK GLPQSTISFD GIYANMRMVH ILTSVVGSKC |
| 121 | EVQVKNGGIY EGVFKTYSPK CDLVLDAAHE KSTESSSGPK REEIMESILF KCSDFVVVQF |
| 181 | KDMDSSYAKR DAFTDSAISA KVNGEHKEKD LEPWDAGELT ANEELEALEN DVSNGWDPND |
| 241 | MFRYNEENYG VVSTYDSSLS SYTVPLERDN SEEFLKREAR ANQLAEEIES SAQYKARVAL |
| 301 | ENDDRSEEEK YTAVQRNSSE REGHSINTRE NKYIPPGQRN REVISWGSGR QNSPRMGQPG |
| 361 | SGSMPSRSTS HTSDFNPNSG SDQRVVNGGV PWPSPCPSPS SRPPSRYQSG PNSLPPRAAT |
| 421 | PTRPPSRPPS RPSRPPSHPS AHGSPAPVST MPKRMSSEGP PRMSPKAQRH PRNHRVSAGR |
| 481 | GSISSGLEFV SHNPPSEAAT PPVARTSPSG GTWSSVVSGV PRLSPKTHRP RSPRQNSIGN |
| 541 | TPSGPVLASP QAGIIPTEAV AMPIPAASPT PASPASNRAV TPSSEAKDSR LQDQRQNSPA |
| 601 | GNKENIKPNE TSPSFSKAEN KGISPVVSEH RKQIDDLKKF KNDFRLQPSS TSESMDQLLN |
| 661 | KNREGEKSRD LIKDKIEPSA KDSFIENSSS NCTSGSSKPN SPSISPSILS NTEHKRGPEV |
| 721 | TSQGVQTSSP ACKQEKDDKE EKKDAAEQVR KSTLNPNAKE FNPRSFSQPK PSTTPTSPRP |
| 781 | QAQPSPSMVG HQQPTPVYTQ PVCFAPNMMY PVPVSPGVQP LYPIPMTPMP VNQAKTYRAV |
| 841 | PNMPQQRQDQ HHQSAMMHPA SAAGPPIAAT PPAYSTQYVA YSPQQFPNQP LVQHVPHYQS |
| 901 | QHPHVYSPVI QGNARMMAPP THAQPGLVSS SATQYGAHEQ THAMYACPKL PYNKETSPSF |
| 961 | YFAISTGSLA QQYAHPNATL HPHTPHPQPS ATPTGQQQSQ HGGSHPAPSP VQHHQHQAAQ |
| 1021 | ALHLASPQQQ SAIYHAGLAP TPPSMTPASN TQSPQNSFPA AQQTVFTIHP SHVQPAYTNP |
| 1081 | PHMAHVPQAH VQSGMVPSHP TAHAPMMLMT TQPPGGPQAA LAQSALQPIP VSTTAHFPYM |
| 1141 | THPSVQAHHQ QQL. |

The human ATXN2 isoform 2 mRNA sequence can be found at NM_001310121.1 (SEQ ID NO: 63); and the corresponding protein sequence can be found at NP_001297050.1 (SEQ ID NO: 64).

| (SEQ ID NO: 63) | |
|---|---|
| 1 | CCCGAGAAAG CAACCCAGCG CGCCGCCCGC TCCTCACGTG TCCCTCCCGG CCCCGGGGCC |
| 61 | ACCTCACGTT CTGCTTCCGT CTGACCCCTC CGACTTCCGA GGTCGAAACA GTAACAAAGG |
| 121 | ACTGCCTCAG TCTACGATTT CTTTTGATGG AATCTATGCA AATATGAGGA TGGTTCATAT |
| 181 | ACTTACATCA GTTGTTGGCT CCAAATGTGA AGTACAAGTG AAAAATGGAG GTATATATGA |
| 241 | AGGAGTTTTT AAAACTTACA GTCCGAAGTG TGATTTGGTA CTTGATGCCG CACATGAGAA |
| 301 | AAGTACAGAA TCCAGTTCGG GGCCGAAACG TGAAGAAATA ATGGAGAGTA TTTTGTTCAA |
| 361 | ATGTTCAGAC TTTGTTGTGG TACAGTTTAA AGATATGGAC TCCAGTTATG CAAAAAGAGA |
| 421 | TGCTTTTACT GACTCTGCTA TCAGTGCTAA AGTGAATGGC GAACACAAAG AGAAGGACCT |
| 481 | GGAGCCCTGG GATGCAGGTG AACTCACAGC CAATGAGGAA CTTGAGGCTT TGGAAAATGA |
| 541 | CGTATCTAAT GGATGGGATC CCAATGATAT GTTTCGATAT AATGAAGAAA ATTATGGTGT |
| 601 | AGTGTCTACG TATGATAGCA GTTTATCTTC GTATACAGTG CCCTTAGAAA GAGATAACTC |
| 661 | AGAAGAATTT TTAAAACGGG AAGCAAGGGC AAACCAGTTA GCAGAAGAAA TTGAGTCAAG |
| 721 | TGCCCAGTAC AAAGCTCGAG TGGCCCTGGA AAATGATGAT AGGAGTGAGG AAGAAAAATA |
| 781 | CACAGCAGTT CAGAGAAATT CCAGTGAACG TGAGGGGCAC AGCATAAACA CTAGGGAAAA |
| 841 | TAAATATATT CCTCCTGGAC AAAGAAATAG AGAAGTCATA TCCTGGGGAA GTGGGAGACA |
| 901 | GAATTCACCG CGTATGGGCC AGCCTGGATC GGGCTCCATG CCATCAAGAT CCACTTCTCA |
| 961 | CACTTCAGAT TTCAACCCGA ATTCTGGTTC AGACCAAAGA GTAGTTAATG GAGGTGTTCC |
| 1021 | CTGGCCATCG CCTTGCCCAT CTCCTTCCTC TCGCCCACCT TCTCGCTACC AGTCAGGTCC |
| 1081 | CAACTCTCTT CCACCTCGGG CAGCCACCCC TACACGGCCG CCCTCCAGGC CCCCCTCGCG |
| 1141 | GCCATCCAGA CCCCCGTCTC ACCCCTCTGC TCATGGTTCT CCAGCTCCTG TCTCTACTAT |
| 1201 | GCCTAAACGC ATGTCTTCAG AAGGGCCTCC AAGGATGTCC CCAAAGGCCC AGCGACATCC |
| 1261 | TCGAAATCAC AGAGTTTCTG CTGGGAGGGG TTCCATATCC AGTGGCCTAG AATTTGTATC |
| 1321 | CCACAACCCA CCCAGTGAAG CAGCTACTCC TCCAGTAGCA AGGACCAGTC CCTCGGGGGG |
| 1381 | AACGTGGTCA TCAGTGGTCA GTGGGGTTCC AAGATTATCC CCTAAAACTC ATAGACCCAG |
| 1441 | GTCTCCCAGA CAGAACAGTA TTGGAAATAC CCCCAGTGGG CCAGTTCTTG CTTCTCCCCA |
| 1501 | AGCTGGTATT ATTCCAACTG AAGCTGTTGC CATGCCTATT CCAGCTGCAT CTCCTACGCC |
| 1561 | TGCTAGTCCT GCATCGAACA GAGCTGTTAC CCCTTCTAGT GAGGCTAAAG ATTCCAGGCT |
| 1621 | TCAAGATCAG AGGCAGAACT CTCCTGCAGG GAATAAAGAA AATATTAAAC CCAATGAAAC |
| 1681 | ATCACCTAGC TTCTCAAAAG CTGAAAACAA AGGTATATCA CCAGTTGTTT CTGAACATAG |
| 1741 | AAAACAGATT GATGATTTAA AGAAATTTAA GAATGATTTT AGGTTACAGC CAAGTTCTAC |
| 1801 | TTCTGAATCT ATGGATCAAC TACTAAACAA AAATAGAGAG GGAGAAAAAT CAAGAGATTT |
| 1861 | GATCAAAGAC AAAATTGAAC CAAGTGCTAA GGATTCTTTC ATTGAAAATA GCAGCAGCAA |
| 1921 | CTGTACCAGT GGCAGCAGCA AGCCGAATAG CCCCAGCATT TCCCCTTCAA TACTTAGTAA |
| 1981 | CACGGAGCAC AAGAGGGGAC CTGAGGTCAC TTCCCAAGGG GTTCAGACTT CCAGCCCAGC |
| 2041 | ATGTAAACAA GAGAAAGACG ATAAGGAAGA GAAGAAAGAC GCAGCTGAGC AAGTTAGGAA |
| 2101 | ATCAACATTG AATCCCAATG CAAAGGAGTT CAACCCACGT TCCTTCTCTC AGCCAAAGCC |
| 2161 | TTCTACTACC CCAACTTCAC CTCGGCCTCA AGCACAACCT AGCCCATCTA TGGTGGGTCA |
| 2221 | TCAACAGCCA ACTCCAGTTT ATACTCAGCC TGTTTGTTTT GCACCAAATA TGATGTATCC |
| 2281 | AGTCCCAGTG AGCCCAGGCG TGCAACCTTT ATACCCAATA CCTATGACGC CCATGCCAGT |
| 2341 | GAATCAAGCC AAGACATATA GAGCAGTACC AAATATGCCC CAACAGCGGC AAGACCAGCA |
| 2401 | TCATCAGAGT GCCATGATGC ACCCAGCGTC AGCAGCGGGC CCACCGATTG CAGCCACCCC |
| 2461 | ACCAGCTTAC TCCACGCAAT ATGTTGCCTA CAGTCCTCAG CAGTTCCCAA ATCAGCCCCT |
| 2521 | TGTTCAGCAT GTGCCACATT ATCAGTCTCA GCATCCTCAT GTCTATAGTC CTGTAATACA |
| 2581 | GGGTAATGCT AGAATGATGG CACCACCAAC ACACGCCCAG CCTGGTTTAG TATCTTCTTC |
| 2641 | AGCAACTCAG TACGGGGCTC ATGAGCAGAC GCATGCGATG TATGCATGTC CCAAATTACC |
| 2701 | ATACAACAAG GAGACAAGCC CTTCTTTCTA CTTTGCCATT TCCACGGGCT CCCTTGCTCA |
| 2761 | GCAGTATGCG CACCCTAACG CTACCCTGCA CCCACATACT CCACACCCTC AGCCTTCAGC |
| 2821 | TACCCCCACT GGACAGCAGC AAAGCCAACA TGGTGGAAGT CATCCTGCAC CCAGTCCTGT |
| 2881 | TCAGCACCAT CAGCACCAGG CCGCCCAGGC TCTCCATCTG GCCAGTCCAC AGCAGCAGTC |
| 2941 | AGCCATTTAC CACGCGGGGC TTGCGCCAAC TCCACCCTCC ATGACACCTG CCTCCAACAC |
| 3001 | GCAGTCGCCA CAGAATAGTT TCCCAGCAGC ACAACAGACT GTCTTTACGA TCCATCCTTC |
| 3061 | TCACGTTCAG CCGGCGTATA CCAACCCACC CCACATGGCC CACGTACCTC AGTGCGCCAG |
| 3121 | TGAGGCTCTG GCAAGGTGTG GGCTAGAGAT GCGACTCAGT TGGATCTATC TCTCAGAAGG |
| 3181 | CTACCTTGCT CATGTACAGT CAGGAATGGT TCCTTCTCAT CCAACTGCCC ATGCGCCAAT |
| 3241 | GATGCTAATG ACGACACAGC CACCCGGCGG TCCCCAGGCC GCCCTCGCTC AAAGTGCACT |
| 3301 | ACAGCCCATT CCAGTCTCGA CAACAGCGCA TTTCCCCTAT ATGACGCACC CTTCAGTACA |
| 3361 | AGCCCACCAC CAACAGCAGT TGTAAGGCTG CCCTGGAGGA ACCGAAAGGC CAAATTCCCT |
| 3421 | CCTCCCTTCT ACTGCTTCTA CCAACTGGAA GCACAGAAAA CTAGAATTTC ATTTATTTTG |
| 3481 | TTTTTAAAAT ATATATGTTG ATTTCTTGTA ACATCCAATA GGAATGCTAA CAGTTCACTT |
| 3541 | GCAGTGGAAG ATACTTGGAC CGAGTAGAGG CATTTAGGAA CTTGGGGGCT ATTCCATAAT |
| 3601 | TCCATATGCT GTTTCAGAGT CCCGCAGGTA CCCCAGCTCT GCTTGCCGAA ACTGGAAGTT |
| 3661 | ATTTATTTTT TAATAACCCT TGAAAGTCAT GAACACATCA GCTAGCAAAA GAAGTAACAA |
| 3721 | GAGTGATTCT TGCTGCTATT ACTGCTAAAA AAAAAAAAAA AAAAAAATCA AGACTTGGAA |
| 3781 | CGCCCTTTTA CTAAACTTGA CAAAGTTTCA GTAAATTCTT ACCGTCAAAC TGACGGATTA |
| 3841 | TTATTTATAA ATCAAGTTTG ATGAGGTGAT CACTGTCTAC AGTGGTTCAA CTTTTAAGTT |
| 3901 | AAGGGAAAAA CTTTTACTTT GTAGATAATA TAAAATAAAA ACTTAAAAAA AATTTAAAAA |
| 3961 | ATAAAAAAAG TTTTAAAAAC TGAAAAAAAA AAA |
| (SEQ ID NO: 64) | |
| 1 | MRMVHILTSV VGSKCEVQVK NGGIYEGVFK TYSPKCDLVL DAAHEKSTES SSGPKREEIM |
| 61 | ESILFKCSDF VVVQFKDMDS SYAKRDAFTD SAISAKVNGE HKEKDLEPWD AGELTANEEL |
| 121 | EALENDVSNG WDPNDMFRYN EENYGVVSTY DSSLSSYTVP LERDNSEEFL KREARANQLA |
| 181 | EEIESSAQYK ARVALENDDR SEEEKYTAVQ RNSSEREGHS INTRENKYIP PGQRNREVIS |
| 241 | WGSGRQNSPR MGQPGSGSMP SRSTSHTSDF NPNSGSDQRV VNGGVPWPSP CPSPSSRPPS |
| 301 | RYQSGPNSLP PRAATPTRPP SRPPSRPSRP PSHPSAHGSP APVSTMPKRM SSEGPPRMSP |
| 361 | KAQRHPRNHR VSAGRGSISS GLEFVSHNPP SEAATPPVAR TSPSGGTWSS VVSGVPRLSP |
| 421 | KTHRPRSPRQ NSIGNTPSGP VLASPQAGII PTEAVAMPIP AASPTPASPA SNRAVTPSSE |
| 481 | AKDSRLQDQR QNSPAGNKEN IKPNETSPSF SKAENKGISP VVSEHRKQID DLKKFKNDFR |
| 541 | LQPSSTSESM DQLLNKNREG EKSRDLIKDK IEPSAKDSFI ENSSSNCTSG SSKPNSPSIS |
| 601 | PSILSNTEHK RGPEVTSQGV QTSSPACKQE KDDKEEKKDA AEQVRKSTLN PNAKEFNPRS |
| 661 | FSQPKPSTTP TSPRPQAQPS PSMVGHQQPT PVYTQPVCFA PNMMYPVPVS PGVQPLYPIP |
| 721 | MTPMPVNQAK TYRAVPNMPQ QRQDQHHQSA MMHPASAAGP PIAATPPAYS TQYVAYSPQQ |
| 781 | FPNQPLVQHV PHYQSQHPHV YSPVIQGNAR MMAPPTHAQP GLVSSSATQY GAHEQTHAMY |
| 841 | ACPKLPYNKE TSPSFYFAIS TGSLAQQYAH PNATLHPHTP HPQPSATPTG QQQSQHGGSH |
| 901 | PAPSPVQHHQ HQAAQALHLA SPQQQSAIYH AGLAPTPPSM TPASNTQSPQ NSFPAAQQTV |
| 961 | FTIHPSHVQP AYTNPPHMAH VPQCASEALA RCGLEMRLSW IYLSEGYLAH VQSGMVPSHP |
| 1021 | TAHAPMMLMT TQPPGGPQAA LAQSALQPIP VSTTAHFPYM THPSVQAHHQ QQL |

The human ATXN2 isoform 3 mRNA sequence can be found at NM_001310123.1 (SEQ ID NO: 65); and the corresponding protein sequence can be found at NP_001297052.1 (SEQ ID NO: 66).

| (SEQ ID NO: 65) | |
|---|---|
| 1 | CCCGAGAAAG CAACCCAGCG CGCCGCCCGC TCCTCACGTG TCCCTCCCGG CCCCGGGGCC |
| 61 | ACCTCACGTT CTGCTTCCGT CTGACCCCTC CGACTTCCGA TTTCTTTTGA TGGAATCTAT |
| 121 | GCAAATATGA GGATGGTTCA TATACTTACA TCAGTTGTTT GTGATTTGGT ACTTGATGCC |
| 181 | GCACATGAGA AAAGTACAGA ATCCAGTTCG GGGCCGAAAC GTGAAGAAAT AATGGAGAGT |
| 241 | ATTTTGTTCA AATGTTCAGA CTTTGTTGTG GTACAGTTTA AAGATATGGA CTCCAGTTAT |
| 301 | GCAAAAAGAG ATGCTTTTAC TGACTCTGCT ATCAGTGCTA AAGTGAATGG CGAACACAAA |
| 361 | GAGAAGGACC TGGAGCCCTG GGATGCAGGT GAACTCACAG CCAATGAGGA ACTTGAGGCT |
| 421 | TTGGAAAATG ACGTATCTAA TGGATGGGAT CCCAATGATA TGTTTCGATA TAATGAAGAA |
| 481 | AATTATGGTG TAGTGTCTAC GTATGATAGC AGTTTATCTT CGTATACAGT GCCCTTAGAA |
| 541 | AGAGATAACT CAGAAGAATT TTTAAAACGG GAAGCAAGGG CAAACCAGTT AGCAGAAGAA |
| 601 | ATTGAGTCAA GTGCCCAGTA CAAAGCTCGA GTGGCCCTGG AAAATGATGA TAGGAGTGAG |
| 661 | GAAGAAAAAT ACACAGCAGT TCAGAGAAAT TCCAGTGAAC GTGAGGGGCA CAGCATAAAC |
| 721 | ACTAGGGAAA ATAAATATAT TCCTCCTGGA CAAAGAAATA GAGAAGTCAT ATCCTGGGGA |
| 781 | AGTGGGAGAC AGAATTCACC GCGTATGGGC CAGCCTGGAT CGGGCTCCAT GCCATCAAGA |
| 841 | TCCACTTCTC ACACTTCAGA TTTCAACCCG AATTCTGGTT CAGACCAAAG AGTAGTTAAT |
| 901 | GGAGGTGTTC CCTGGCCATC GCCTTGCCCA TCTCCTTCCT CTCGCCCACC TTCTCGCTAC |
| 961 | CAGTCAGGTC CCAACTCTCT TCCACCTCGG GCAGCCACCC CTACACGGCC GCCCTCCAGG |
| 1021 | CCCCCCTCGC GGCCATCCAG ACCCCCGTCT CACCCCTCTG CTCATGGTTC TCCAGCTCCT |
| 1081 | GTCTCTACTA TGCCTAAACG CATGTCTTCA GAAGGGCCTC CAAGGATGTC CCCAAAGGCC |
| 1141 | CAGCGACATC CTCGAAATCA CAGAGTTTCT GCTGGGAGGG GTTCCATATC CAGTGGCCTA |
| 1201 | GAATTTGTAT CCCACAACCC ACCCAGTGAA GCAGCTACTC CTCCAGTAGC AAGGACCAGT |
| 1261 | CCCTCGGGGG GAACGTGGTC ATCAGTGGTC AGTGGGGTTC CAAGATTATC CCCTAAAACT |
| 1321 | CATAGACCCA GGTCTCCCAG ACAGAACAGT ATTGGAAATA CCCCCAGTGG GCCAGTTCTT |
| 1381 | GCTTCTCCCC AAGCTGGTAT TATTCCAACT GAAGCTGTTG CCATGCCTAT TCCAGCTGCA |
| 1441 | TCTCCTACGC CTGCTAGTCC TGCATCGAAC AGAGCTGTTA CCCCTTCTAG TGAGGCTAAA |
| 1501 | GATTCCAGGC TTCAAGATCA GAGGCAGAAC TCTCCTGCAG GGAATAAAGA AAATATTAAA |
| 1561 | CCCAATGAAA CATCACCTAG CTTCTCAAAA GCTGAAAACA AAGGTATATC ACCAGTTGTT |
| 1621 | TCTGAACATA GAAAACAGAT TGATGATTTA AAGAAATTTA AGAATGATTT TAGGTTACAG |
| 1681 | CCAAGTTCTA CTTCTGAATC TATGGATCAA CTACTAAACA AAAATAGAGA GGGAGAAAAA |
| 1741 | TCAAGAGATT TGATCAAAGA CAAAATTGAA CCAAGTGCTA AGGATTCTTT CATTGAAAAT |
| 1801 | AGCAGCAGCA ACTGTACCAG TGGCAGCAGC AAGCCGAATA GCCCCAGCAT TTCCCCTTCA |
| 1861 | ATACTTAGTA ACACGGAGCA CAAGAGGGGA CCTGAGGTCA CTTCCCAAGG GGTTCAGACT |
| 1921 | TCCAGCCCAG CATGTAAACA AGAGAAAGAC GATAAGGAAG AGAAGAAAGA CGCAGCTGAG |
| 1981 | CAAGTTAGGA AATCAACATT GAATCCCAAT GCAAAGGAGT TCAACCCACG TTCCTTCTCT |
| 2041 | CAGCCAAAGC CTTCTACTAC CCCAACTTCA CCTCGGCCTC AAGCACAACC TAGCCCATCT |
| 2101 | ATGGTGGGTC ATCAACAGCC AACTCCAGTT TATACTCAGC CTGTTTGTTT TGCACCAAAT |
| 2161 | ATGATGTATC CAGTCCCAGT GAGCCCAGGC GTGCAACCTT TATACCCAAT ACCTATGACG |
| 2221 | CCCATGCCAG TGAATCAAGC CAAGACATAT AGAGCAGTAC CAAATATGCC CCAACAGCGG |
| 2281 | CAAGACCAGC ATCATCAGAG TGCCATGATG CACCCAGCGT CAGCAGCGGG CCCACCGATT |
| 2341 | GCAGCCACCC CACCAGCTTA CTCCACGCAA TATGTTGCCT ACAGTCCTCA GCAGTTCCCA |
| 2401 | AATCAGCCCC TTGTTCAGCA TGTGCCACAT TATCAGTCTC AGCATCCTCA TGTCTATAGT |
| 2461 | CCTGTAATAC AGGGTAATGC TAGAATGATG GCACCACCAA CACACGCCCA GCCTGGTTTA |
| 2521 | GTATCTTCTT CAGCAACTCA GTACGGGGCT CATGAGCAGA CGCATGCGAT GTATGTTTCC |
| 2581 | ACGGGCTCCC TTGCTCAGCA GTATGCGCAC CCTAACGCTA CCCTGCACCC ACATACTCCA |
| 2641 | CACCCTCAGC CTTCAGCTAC CCCCACTGGA CAGCAGCAAA GCCAACATGG TGGAAGTCAT |
| 2701 | CCTGCACCCA GTCCTGTTCA GCACCATCAG CACCAGGCCG CCCAGGCTCT CCATCTGGCC |
| 2761 | AGTCCACAGC AGCAGTCAGC CATTTACCAC GCGGGGCTTG CGCCAACTCC ACCCTCCATG |
| 2821 | ACACCTGCCT CCAACACGCA GTCGCCACAG AATAGTTTCC CAGCAGCACA ACAGACTGTC |
| 2881 | TTTACGATCC ATCCTTCTCA CGTTCAGCCG GCGTATACCA ACCCACCCCA CATGGCCCAC |
| 2941 | GTACCTCAGG CTCATGTACA GTCAGGAATG GTTCCTTCTC ATCCAACTGC CCATGCGCCA |
| 3001 | ATGATGCTAA TGACGACACA GCCACCCGGC GGTCCCCAGG CCGCCCTCGC TCAAAGTGCA |
| 3061 | CTACAGCCCA TTCCAGTCTC GACAACAGCG CATTTCCCCT ATATGACGCA CCCTTCAGTA |
| 3121 | CAAGCCCACC ACCAACAGCA GTTGTAAGGC TGCCCTGGAG GAACCGAAAG GCCAAATTCC |
| 3181 | CTCCTCCCTT CTACTGCTTC TACCAACTGG AAGCACAGAA AACTAGAATT TCATTTATTT |
| 3241 | TGTTTTTAAA ATATATATGT TGATTTCTTG TAACATCCAA TAGGAATGCT AACAGTTCAC |
| 3301 | TTGCAGTGGA AGATACTTGG ACCGAGTAGA GGCATTTAGG AACTTGGGGG CTATTCCATA |
| 3361 | ATTCCATATG CTGTTTCAGA GTCCCGCAGG TACCCCAGCT CTGCTTGCCG AAACTGGAAG |
| 3421 | TTATTTATTT TTTAATAACC CTTGAAAGTC ATGAACACAT CAGCTAGCAA AAGAAGTAAC |
| 3481 | AAGAGTGATT CTTGCTGCTA TTACTGCTAA AAAAAAAAAA AAAAAAAAAT CAAGACTTGG |
| 3541 | AACGCCCTTT TACTAAACTT GACAAAGTTT CAGTAAATTC TTACCGTCAA ACTGACGGAT |
| 3601 | TATTATTTAT AAATCAAGTT TGATGAGGTG ATCACTGTCT ACAGTGGTTC AACTTTTAAG |
| 3661 | TTAAGGGAAA AACTTTTACT TTGTAGATAA TATAAAATAA AAACTTAAAA AAAATTTAAA |
| 3721 | AAATAAAAAA AGTTTTAAAA ACTGAAAAAA AAAAA |
| (SEQ ID NO: 66) | |
| 1 | MRMVHILTSV VCDLVLDAAH EKSTESSSGP KREEIMESIL FKCSDFVVVQ FKDMDSSYAK |
| 61 | RDAFTDSAIS AKVNGEHKEK DLEPWDAGEL TANEELEALE NDVSNGWDPN DMFRYNEENY |
| 121 | GVVSTYDSSL SSYTVPLERD NSEEFLKREA RANQLAEEIE SSAQYKARVA LENDDRSEEE |
| 181 | KYTAVQRNSS EREGHSINTR ENKYIPPGQR NREVISWGSG RQNSPRMGQP GSGSMPSRST |
| 241 | SHTSDFNPNS GSDQRVVNGG VPWPSPCPSP SSRPPSRYQS GPNSLPPRAA TPTRPPSRPP |
| 301 | SRPSRPPSHP SAHGSPAPVS TMPKRMSSEG PPRMSPKAQR HPRNHRVSAG RGSISSGLEF |
| 361 | VSHNPPSEAA TPPVARTSPS GGTWSSVVSG VPRLSPKTHR PRSPRQNSIG NTPSGPVLAS |
| 421 | PQAGIIPTEA VAMPIPAASP TPASPASNRA VTPSSEAKDS RLQDQRQNSP AGNKENIKPN |
| 481 | ETSPSFSKAE NKGISPVVSE HRKQIDDLKK FKNDFRLQPS STSESMDQLL NKNREGEKSR |
| 541 | DLIKDKIEPS AKDSFIENSS SNCTSGSSKP NSPSISPSIL SNTEHKRGPE VTSQGVQTSS |
| 601 | PACKQEKDDK EEKKDAAEQV RKSTLNPNAK EFNPRSFSQP KPSTTPTSPR PQAQPSPSMV |
| 661 | GHQQPTPVYT QPVCFAPNMM YPVPVSPGVQ PLYPIPMTPM PVNQAKTYRA VPNMPQQRQD |
| 721 | QHHQSAMMHP ASAAGPPIAA TPPAYSTQYV AYSPQQFPNQ PLVQHVPHYQ SQHPHVYSPV |
| 781 | IQGNARMMAP PTHAQPGLVS SSATQYGAHE QTHAMYVSTG SLAQQYAHPN ATLHPHTPHP |
| 841 | QPSATPTGQQ QSQHGGSHPA PSPVQHHQHQ AAQALHLASP QQQSAIYHAG LAPTPPSMTP |
| 901 | ASNTQSPQNS FPAAQQTVFT IHPSHVQPAY TNPPHMAHVP QAHVQSGMVP SHPTAHAPMM |
| 961 | LMTTQPPGGP QAALAQSALQ PIPVSTTAHF PYMTHPSVQA HHQQQL |

The human ATXN2 isoform 4 mRNA sequence can be found at NM_001372574.1 (SEQ ID NO: 67); the corresponding protein sequence can be found at NP_001359503.1 (SEQ ID NO: 68).

| (SEQ ID NO: 67) | |
|---|---|
| 1 | AGAGCTCGCC TCCCTCCGCC TCAGACTGTT TTGGTAGCAA CGGCAACGGC GGCGGCGCGT |
| 61 | TTCGGCCCGG CTCCCGGCGG CTCCTTGGTC TCGGCGGGCC TCCCCGCCCC TTCGTCGTCC |
| 121 | TCCTTCTCCC CCTCGCCAGC CCGGGCGCCC CTCCGGCCGC GCCAACCCGC GCCTCCCCGC |
| 181 | TCGGCGCCCG CGCGTCCCCG CCGCGTTCCG GCGTCTCCTT GGCGCGCCCG GCTCCCGGCT |
| 241 | GTCCCCGCCC GGCGTGCGAG CCGGTGTATG GGCCCCTCAC CATGTCGCTG AAGCCCCAGC |
| 301 | AGCAGCAGCA GCAGCAGCAG CAGCAGCAGC AGCAGCAACA GCAGCAGCAG CAGCAGCAGC |
| 361 | AGCAGCCGCC GCCCGCGGCT GCCAATGTCC GCAAGCCCGG CGGCAGCGGC CTTCTAGCGT |
| 421 | CGCCCGCCGC CGCGCCTTCG CCGTCCTCGT CCTCGGTCTC CTCGTCCTCG GCCACGGCTC |
| 481 | CCTCCTCGGT GGTCGCGGCG ACCTCCGGCG GCGGGAGGCC CGGCCTGGGC AGAGGTCGAA |
| 541 | ACAGTAACAA AGGACTGCCT CAGTCTACGA TTTCTTTTGA TGGAATCTAT GCAAATATGA |
| 601 | GGATGGTTCA TATACTTACA TCAGTTGTTG GCTCCAAATG TGAAGTACAA GTGAAAAATG |
| 661 | GAGGTATATA TGAAGGAGTT TTTAAAACTT ACAGTCCGAA GTGTGATTTG GTACTTGATG |
| 721 | CCGCACATGA GAAAAGTACA GAATCCAGTT CGGGGCCGAA ACGTGAAGAA ATAATGGAGA |
| 781 | GTATTTTGTT CAAATGTTCA GACTTTGTTG TGGTACAGTT TAAAGATATG GACTCCAGTT |
| 841 | ATGCAAAAAG AGATGCTTTT ACTGACTCTG CTATCAGTGC TAAAGTGAAT GGCGAACACA |
| 901 | AAGAGAAGGA CCTGGAGCCC TGGGATGCAG GTGAACTCAC AGCCAATGAG GAACTTGAGG |
| 961 | CTTTGGAAAA TGACGTATCT AATGGATGGG ATCCCAATGA TATGTTTCGA TATAATGAAG |
| 1021 | AAAATTATGG TGTAGTGTCT ACGTATGATA GCAGTTTATC TTCGTATACA GTGCCCTTAG |
| 1081 | AAAGAGATAA CTCAGAAGAA TTTTTAAAAC GGGAAGCAAG GGCAAACCAG TTAGCAGAAG |
| 1141 | AAATTGAGTC AAGTGCCCAG TACAAAGCTC GAGTGGCCCT GGAAAATGAT GATAGGAGTG |
| 1201 | AGGAAGAAAA ATACACAGCA GTTCAGAGAA ATTCCAGTGA ACGTGAGGGG CACAGCATAA |
| 1261 | ACACTAGGGA AAATAAATAT ATTCCTCCTG GACAAAGAAA TAGAGAAGTC ATATCCTGGG |
| 1321 | GAAGTGGGAG ACAGAATTCA CCGCGTATGG GCCAGCCTGG ATCGGGCTCC ATGCCATCAA |
| 1381 | GATCCACTTC TCACACTTCA GATTTCAACC CGAATTCTGG TTCAGACCAA AGAGTAGTTA |
| 1441 | ATGGAGGTGT TCCCTGGCCA TCGCCTTGCC CATCTCCTTC CTCTCGCCCA CCTTCTCGCT |
| 1501 | ACCAGTCAGG TCCCAACTCT CTTCCACCTC GGGCAGCCAC CCCTACACGG CCGCCCTCCA |
| 1561 | GGCCCCCCTC GCGGCCATCC AGACCCCCGT CTCACCCCTC TGCTCATGGT TCTCCAGCTC |
| 1621 | CTGTCTCTAC TATGCCTAAA CGCATGTCTT CAGAAGGGCC TCCAAGGATG TCCCCAAAGG |
| 1681 | CCCAGCGACA TCCTCGAAAT CACAGAGTTT CTGCTGGGAG GGGTTCCATA TCCAGTGGCC |
| 1741 | TAGAATTTGT ATCCCACAAC CCACCCAGTG AAGCAGCTAC TCCTCCAGTA GCAAGGACCA |
| 1801 | GTCCCTCGGG GGGAACGTGG TCATCAGTGG TCAGTGGGGT TCCAAGATTA TCCCCTAAAA |
| 1861 | CTCATAGACC CAGGTCTCCC AGACAGAACA GTATTGGAAA TACCCCCAGT GGGCCAGTTC |
| 1921 | TTGCTTCTCC CCAAGCTGGT ATTATTCCAA CTGAAGCTGT TGCCATGCCT ATTCCAGCTG |
| 1981 | CATCTCCTAC GCCTGCTAGT CCTGCATCGA ACAGAGCTGT TACCCCTTCT AGTGAGGCTA |
| 2041 | AAGATTCCAG GCTTCAAGAT CAGAGGCAGA ACTCTCCTGC AGGGAATAAA GAAAATATTA |
| 2101 | AACCCAATGA AACATCACCT AGCTTCTCAA AAGCTGAAAA CAAAGGTATA TCACCAGTTG |
| 2161 | TTTCTGAACA TAGAAAACAG ATTGATGATT TAAAGAAATT TAAGAATGAT TTTAGGTTAC |
| 2221 | AGCCAAGTTC TACTTCTGAA TCTATGGATC AACTACTAAA CAAAAATAGA GAGGGAGAAA |
| 2281 | AATCAAGAGA TTTGATCAAA GACAAAATTG AACCAAGTGC TAAGGATTCT TTCATTGAAA |
| 2341 | ATAGCAGCAG CAACTGTACC AGTGGCAGCA GCAAGCCGAA TAGCCCCAGC ATTTCCCCTT |
| 2401 | CAATACTTAG TAACACGGAG CACAAGAGGG GACCTGAGGT CACTTCCCAA GGGGTTCAGA |
| 2461 | CTTCCAGCCC AGCATGTAAA CAAGAGAAAG ACGATAAGGA AGAGAAGAAA GACGCAGCTG |
| 2521 | AGCAAGTTAG GAAATCAACA TTGAATCCCA ATGCAAAGGA GTTCAACCCA CGTTCCTTCT |
| 2581 | CTCAGCCAAA GCCTTCTACT ACCCCAACTT CACCTCGGCC TCAAGCACAA CCTAGCCCAT |
| 2641 | CTATGGTGGG TCATCAACAG CCAACTCCAG TTTATACTCA GCCTGTTTGT TTTGCACCAA |
| 2701 | ATATGATGTA TCCAGTCCCA GTGAGCCCAG GCGTGCAACC TTTATACCCA ATACCTATGA |
| 2761 | CGCCCATGCC AGTGAATCAA GCCAAGACAT ATAGAGCAGG TAAAGTACCA AATATGCCCC |
| 2821 | AACAGCGGCA AGACCAGCAT CATCAGAGTG CCATGATGCA CCCAGCGTCA GCAGCGGGCC |
| 2881 | CACCGATTGC AGCCACCCCA CCAGCTTACT CCACGCAATA TGTTGCCTAC AGTCCTCAGC |
| 2941 | AGTTCCCAAA TCAGCCCCTT GTTCAGCATG TGCCACATTA TCAGTCTCAG CATCCTCATG |
| 3001 | TCTATAGTCC TGTAATACAG GGTAATGCTA GAATGATGGC ACCACCAACA CACGCCCAGC |
| 3061 | CTGGTTTAGT ATCTTCTTCA GCAACTCAGT ACGGGGCTCA TGAGCAGACG CATGCGATGT |
| 3121 | ATGCATGTCC CAAATTACCA TACAACAAGG AGACAAGCCC TTCTTTCTAC TTTGCCATTT |
| 3181 | CCACGGGCTC CCTTGCTCAG CAGTATGCGC ACCCTAACGC TACCCTGCAC CCACATACTC |
| 3241 | CACACCCTCA GCCTTCAGCT ACCCCCACTG GACAGCAGCA AAGCCAACAT GGTGGAAGTC |
| 3301 | ATCCTGCACC CAGTCCTGTT CAGCACCATC AGCACCAGGC CGCCCAGGCT CTCCATCTGG |
| 3361 | CCAGTCCACA GCAGCAGTCA GCCATTTACC ACGCGGGGCT TGCGCCAACT CCACCCTCCA |
| 3421 | TGACACCTGC CTCCAACACG CAGTCGCCAC AGAATAGTTT CCCAGCAGCA CAACAGACTG |
| 3481 | TCTTTACGAT CCATCCTTCT CACGTTCAGC CGGCGTATAC CAACCCACCC CACATGGCCC |
| 3541 | ACGTACCTCA GGCTCATGTA CAGTCAGGAA TGGTTCCTTC TCATCCAACT GCCCATGCGC |
| 3601 | CAATGATGCT AATGACGACA CAGCCACCCG GCGGTCCCCA GGCCGCCCTC GCTCAAAGTG |
| 3661 | CACTACAGCC CATTCCAGTC TCGACAACAG CGCATTTCCC CTATATGACG CACCCTTCAG |
| 3721 | TACAAGCCCA CCACCAACAG CAGTTGTAAG GCTGCCCTGG AGGAACCGAA AGGCCAAATT |
| 3781 | CCCTCCTCCC TTCTACTGCT TCTACCAACT GGAAGCACAG AAAACTAGAA TTTCATTTAT |
| 3841 | TTTGTTTTTA AAATATATAT GTTGATTTCT TGTAACATCC AATAGGAATG CTAACAGTTC |
| 3901 | ACTTGCAGTG GAAGATACTT GGACCGAGTA GAGGCATTTA GGAACTTGGG GGCTATTCCA |
| 3961 | TAATTCCATA TGCTGTTTCA GAGTCCCGCA GGTACCCCAG CTCTGCTTGC CGAAACTGGA |
| 4021 | AGTTATTTAT TTTTTAATAA CCCTTGAAAG TCATGAACAC ATCAGCTAGC AAAAGAAGTA |
| 4081 | ACAAGAGTGA TTCTTGCTGC TATTACTGCT AAAAAAAAAA AAAAAAAAAA ATCAAGACTT |
| 4141 | GGAACGCCCT TTTACTAAAC TTGACAAAGT TTCAGTAAAT TCTTACCGTC AAACTGACGG |
| 4201 | ATTATTATTT ATAAATCAAG TTTGATGAGG TGATCACTGT CTACAGTGGT TCAACTTTTA |
| 4261 | AGTTAAGGGA AAAACTTTTA CTTTGTAGAT AATATAAAAT AAAAACTTAA AAAAAATTTA |
| 4321 | AAAAATAAAA AAAGTTTTAA AAACTGA |
| (SEQ ID NO: 68) | |
| 1 | MSLKPQQQQQ QQQQQQQQQQ QQQQQQQQPP PAAANVRKPG GSGLLASPAA APSPSSSSVS |
| 61 | SSSATAPSSV VAATSGGGRP GLGRGRNSNK GLPQSTISFD GIYANMRMVH ILTSVVGSKC |
| 121 | EVQVKNGGIY EGVFKTYSPK CDLVLDAAHE KSTESSSGPK REEIMESILF KCSDFVVVQF |
| 181 | KDMDSSYAKR DAFTDSAISA KVNGEHKEKD LEPWDAGELT ANEELEALEN DVSNGWDPND |
| 241 | MFRYNEENYG VVSTYDSSLS SYTVPLERDN SEEFLKREAR ANQLAEEIES SAQYKARVAL |
| 301 | ENDDRSEEEK YTAVQRNSSE REGHSINTRE NKYIPPGQRN REVISWGSGR QNSPRMGQPG |
| 361 | SGSMPSRSTS HTSDFNPNSG SDQRVVNGGV PWPSPCPSPS SRPPSRYQSG PNSLPPRAAT |
| 421 | PTRPPSRPPS RPSRPPSHPS AHGSPAPVST MPKRMSSEGP PRMSPKAQRH PRNHRVSAGR |
| 481 | GSISSGLEFV SHNPPSEAAT PPVARTSPSG GTWSSVVSGV PRLSPKTHRP RSPRQNSIGN |
| 541 | TPSGPVLASP QAGIIPTEAV AMPIPAASPT PASPASNRAV TPSSEAKDSR LQDQRQNSPA |
| 601 | GNKENIKPNE TSPSFSKAEN KGISPVVSEH RKQIDDLKKF KNDFRLQPSS TSESMDQLLN |
| 661 | KNREGEKSRD LIKDKIEPSA KDSFIENSSS NCTSGSSKPN SPSISPSILS NTEHKRGPEV |
| 721 | TSQGVQTSSP ACKQEKDDKE EKKDAAEQVR KSTLNPNAKE FNPRSFSQPK PSTTPTSPRP |
| 781 | QAQPSPSMVG HQQPTPVYTQ PVCFAPNMMY PVPVSPGVQP LYPIPMTPMP VNQAKTYRAG |
| 841 | KVPNMPQQRQ DQHHQSAMMH PASAAGPPIA ATPPAYSTQY VAYSPQQFPN QPLVQHVPHY |
| 901 | QSQHPHVYSP VIQGNARMMA PPTHAQPGLV SSSATQYGAH EQTHAMYACP KLPYNKETSP |
| 961 | SFYFAISTGS LAQQYAHPNA TLHPHTPHPQ PSATPTGQQQ SQHGGSHPAP SPVQHHQHQA |
| 1021 | AQALHLASPQ QQSAIYHAGL APTPPSMTPA SNTQSPQNSF PAAQQTVFTI HPSHVQPAYT |
| 1081 | NPPHMAHVPQ AHVQSGMVPS HPTAHAPMML MTTQPPGGPQ AALAQSALQP IPVSTTAHFP |
| 1141 | YMTHPSVQAH HQQQL |

Another human ATXN2 isoform (that includes UTRs) mRNA sequence (SEQ ID NO: 69) and the corresponding protein sequence (SEQ ID NO: 70) can be found at ENST00000608853.5.

(SEQ ID NO: 69)
ATGTCGCTGAAGCCCCAGCAGCAGCAGCAGCAGCAGCAGCAGCAGCAGCAGCAGCAACAGCAGCAGC
AGCAGCAGCAGCAGCAGCCGCCGCCCGCGGCTGCCAATGTCCGCAAGCCCGGCGGCAGCGGCCTTCT
AGCGTCGCCCGCCGCCGCGCCTTCGCCGTCCTCGTCCTCGGTCTCCTCGTCCTCGGCCACGGCTCCCTC
CTCGGTGGTCGCGGCGACCTCCGGCGGGGGAGGCCCGGCCTGGGCAGAGGTCGAAACAGTAACAAA
GGACTGCCTCAGTCTACGATTTCTTTTGATGGAATCTATGCAAATATGAGGATGGTTCATATACTTACA
TCAGTTGTTGGCTCCAAATGTGAAGTACAAGTGAAAAATGGAGGTATATATGAAGGAGTTTTTAAAAC
TTACAGTCCGAAGTGTGATTTGGTACTTGATGCCGCACATGAGAAAAGTACAGAATCCAGTTCGGGGC
CGAAACGTGAAGAAATAATGGAGAGTATTTTGTTCAAATGTTCAGACTTTGTTGTGGTACAGTTTAAA
GATATGGACTCCAGTTATGCAAAAAGAGATGCTTTTACTGACTCTGCTATCAGTGCTAAAGTGAATGG
CGAACACAAAGAGAAGGACCTGGAGCCCTGGGATGCAGGTGAACTCACAGCCAATGAGGAACTTGAG
GCTTTGGAAAATGACGTATCTAATGGATGGGATCCCAATGATATGTTTCGATATAATGAAGAAAATTA
TGGTGTAGTGTCTACGTATGATAGCAGTTTATCTTCGTATACAGTGCCCTTAGAAAGAGATAACTCAGA
AGAATTTTTAAAACGGGAAGCAAGGGCAAACCAGTTAGCAGAAGAAATTGAGTCAAGTGCCCAGTAC
AAAGCTCGAGTGGCCCTGGAAAATGATGATAGGAGTGAGGAAGAAAAATACACAGCAGTTCAGAGAA
ATTCCAGTGAACGTGAGGGGCACAGCATAAACACTAGGGAAAATAAATATATTCCTCCTGGACAAAG
AAATAGAGAAGTCATATCCTGGGGAAGTGGGAGACAGAATTCACCGCGTATGGGCCAGCCTGGATCG
GGCTCCATGCCATCAAGATCCACTTCTCACACTTCAGATTTCAACCCGAATTCTGGTTCAGACCAAAGA
GTAGTTAATGGAGGTGTTCCCTGGCCATCGCCTTGCCCATCTCCTTCCTCTCGCCCACCTTCTCGCTACC
AGTCAGGTCCCAACTCTCTTCCACCTCGGGCAGCCACCCCTACACGGCCGCCCTCCAGGCCCCCCTCGC
GGCCATCCAGACCCCCGTCTCACCCCTCTGCTCATGGTTCTCCAGCTCCTGTCTCTACTATGCCTAAAC
GCATGTCTTCAGAAGGGCCTCCAAGGATGTCCCCAAAGGCCCAGCGACATCCTCGAAATCACAGAGTT
TCTGCTGGGAGGGGTTCCATATCCAGTGGCCTAGAATTTGTATCCCACAACCCACCCAGTGAAGCAGC
TACTCCTCCAGTAGCAAGGACCAGTCCCTCGGGGGGAACGTGGTCATCAGTGGTCAGTGGGGTTCCAA
GATTATCCCCTAAAACTCATAGACCCAGGTCTCCCAGACAGAACAGTATTGGAAATACCCCCAGTGGG
CCAGTTCTTGCTTCTCCCCAAGCTGGTATTATTCCAACTGAAGCTGTTGCCATGCCTATTCCAGCTGCAT
CTCCTACGCCTGCTAGTCCTGCATCGAACAGAGCTGTTACCCCTTCTAGTGAGGCTAAAGATTCCAGGC
TTCAAGATCAGAGGCAGAACTCTCCTGCAGGGAATAAAGAAAATATTAAACCCAATGAAACATCACC
TAGCTTCTCAAAAGCTGAAAACAAAGGTATATCACCAGTTGTTTCTGAACATAGAAAACAGATTGATG
ATTTAAAGAAATTTAAGAATGATTTTAGGTTACAGCCAAGTTCTACTTCTGAATCTATGGATCAACTAC
TAAACAAAAATAGAGAGGGAGAAAAATCAAGAGATTTGATCAAAGACAAAATTGAACCAAGTGCTAA
GGATTCTTTCATTGAAAATAGCAGCAGCAACTGTACCAGTGGCAGCAGCAAGCCGAATAGCCCCAGCA
TTTCCCCTTCAATACTTAGTAACACGGAGCACAAGAGGGGACCTGAGGTCACTTCCCAAGGGGTTCAG
ACTTCCAGCCCAGCATGTAAACAAGAGAAAGACGATAAGGAAGAGAAGAAAGACGCAGCTGAGCAA
GTTAGGAAATCAACATTGAATCCCAATGCAAAGGAGTTCAACCCACGTTCCTTCTCTCAGCCAAAGCC
TTCTACTACCCCAACTTCACCTCGGCCTCAAGCACAACCTAGCCCATCTATGGTGGGTCATCAACAGCC
AACTCCAGTTTATACTCAGCCTGTTTGTTTTGCACCAAATATGATGTATCCAGTCCCAGTGAGCCCAGG
CGTGCAACCTTTATACCCAATACCTATGACGCCCATGCCAGTGAATCAAGCCAAGACATATAGAGCAG
TACCAAATATGCCCCAACAGCGGCAAGACCAGCATCATCAGAGTGCCATGATGCACCCAGCGTCAGC
AGCGGGCCCACCGATTGCAGCCACCCCACCAGCTTACTCCACGCAATATGTTGCCTACAGTCCTCAGC
AGTTCCCAAATCAGCCCCTTGTTCAGCATGTGCCACATTATCAGTCTCAGCATCCTCATGTCTATAGTC
CTGTAATACAGGGTAATGCTAGAATGATGGCACCACCAACACACGCCCAGCCTGGTTTAGTATCTTCT
TCAGCAACTCAGTACGGGGCTCATGAGCAGACGCATGCGATGTATGCATGTCCCAAATTACCATACAA
CAAGGAGACAAGCCCTTCTTTCTACTTTGCCATTTCCACGGGCTCCCTTGCTCAGCAGTATGCGCACCC
TAACGCTACCCTGCACCCACATACTCCACACCCTCAGCCTTCAGCTACCCCCACTGGACAGCAGCAAA
GCCAACATGGTGGAAGTCATCCTGCACCCAGTCCTGTTCAGCACCATCAGCACCAGGCCGCCCAGGCT
CTCCATCTGGCCAGTCCACAGCAGCAGTCAGCCATTTACCACGCGGGGCTTGCGCCAACTCCACCCTC
CATGACACCTGCCTCCAACACGCAGTCGCCACAGAATAGTTTCCCAGCAGCACAACAGACTGTCTTTA
CGATCCATCCTTCTCACGTTCAGCCGGCGTATACCAACCCACCCCACATGGCCCACGTACCTCAGGCTC
ATGTACAGTCAGGAATGGTTCCTTCTCATCCAACTGCCCATGCGCCAATGATGCTAATGACGACACAG
CCACCCGGCGGTCCCCAGGCCGCCCTCGCTCAAAGTGCACTACAGCCCATTCCAGTCTCGACAACAGC
GCATTTCCCCTATATGACGCACCCTTCAGTACAAGCCCACCACCAACAGCAGTTGTAA
(SEQ ID NO: 70)
MSLKPQQQQQQQQQQQQQQQQQQQQQQQQPPPAAANVRKPGGSGLLASPAAAPSPSSSSVSSSSATAPSSV
VAATSGGGRPGLGRGRNSNKGLPQSTISFDGIYANMRMVHILTSVVGSKCEVQVKNGGIYEGVFKTYSPKC
DLVLDAAHEKSTESSSGPKREEIMESILFKCSDFVVVQFKDMDSSYAKRDAFTDSAISAKVNGEHKEKDLEP
WDAGELTANEELEALENDVSNGWDPNDMFRYNEENYGVVSTYDSSLSSYTVPLERDNSEEFLKREARAN
QLAEEIESSAQYKARVALENDDRSEEEKYTAVQRNSSEREGHSINTRENKYIPPGQRNREVISWGSGRQNSP
RMGQPGSGSMPSRSTSHTSDFNPNSGSDQRVVNGGVPWPSPCPSPSSRPPSRYQSGPNSLPPRAATPTRPPSR
PPSRPSRPPSHPSAHGSPAPVSTMPKRMSSEGPPRMSPKAQRHPRNHRVSAGRGSISSGLEFVSHNPPSEAAT
PPVARTSPSGGTWSSVVSGVPRLSPKTHRPRSPRQNSIGNTPSGPVLASPQAGIIPTEAVAMPIPAASPTPASP
ASNRAVTPSSEAKDSRLQDQRQNSPAGNKENIKPNETSPSFSKAENKGISPVVSEHRKQIDDLKKFKNDFRL
QPSSTSESMDQLLNKNREGEKSRDLIKDKIEPSAKDSFIENSSSNCTSGSSKPNSPSISPSILSNTEHKRGPEV
TSQGVQTSSPACKQEKDDKEEKKDAAEQVRKSTLNPNAKEFNPRSFSQPKPSTTPTSPRPQAQPSPSMVGHQ
QPTPVYTQPVCFAPNMMYPVPVSPGVQPLYPIPMTPMPVNQAKTYRAVPNMPQQRQDQHHQSAMMHPAS
AAGPPIAATPPAYSTQYVAYSPQQFPNQPLVQHVPHYQSQHPHVYSPVIQGNARMMAPPTHAQPGLVSSSA
TQYGAHEQTHAMYACPKLPYNKETSPSFYFAISTGSLAQQYAHPNATLHPHTPHPQPSATPTGQQQSQHGG
SHPAPSPVQHHQHQAAQALHLASPQQQSAIYHAGLAPTPPSMTPASNTQSPQNSFPAAQQTVFTIHPSHVQP
AYTNPPHMAHVPQAHVQSGMVPSHPTAHAPMMLMTTQPPGGPQAALAQSALQPIPVSTTAHFPYMTHPS
VQAHHQQQL.

As used herein, “ATXN2-associated neurological disease” means a neurological disease associated with abnormal ATXN2 expression, activity, or function, CAG repeat or polyglutamine expansion in ATXN2 gene, or abnormal TDP-43 aggregation.

As used herein, “subject” means a mammal, including cat, dog, mouse, rat, chimpanzee, ape, monkey, and human. Preferably the subject is a human.

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

### EXAMPLES

### Example 1. Synthesis of Linker-Delivery Moiety Pairs

Certain abbreviations are defined as follows: “ACN” refers to acetonitrile; “AEX” refers to anion exchange; “C/D” refers to cleavage and deprotection; “CPG” refers to controlled pore glass; “DCM” refers to dichloromethane; “DEA” refers to diethylamine; “DIEA” refers to N, N-diisopropylethylamine; “DMAP” refers to 4-dimethylaminopyridine; “DMF” refers to dimethylformamide; “DMSO” refers to dimethyl sulfoxide; “DMTCl” refers to 4,4′-dimethoxytrityl chloride; “ES/MS” refers to electrospray mass spectrometry; “EtOAc” refers to ethyl acetate; “EtOH” refers to ethanol and ethyl alcohol; “HBTU” refers to 3-[bis(dimethylamino)methyliumyl]-3H-benzotriazol-1-oxide hexafluorophosphate; “HOBt” refers to 1-hydroxybenzotriazole; “IP-RP” refers to ion-pair reverse phase; “LCAA CPG” refers to long chain alkylamine controlled pore glass; “LC/MS” refers to liquid chromatography-mass spectrometry; “MeOH” refers to methanol and methyl alcohol; “MPA” refers to mobile phase A; “MPB” refers to mobile phase B; “MWCO” refers to molecular weight cut-off; “NMR” refers to nuclear magnetic resonance; “PBS” phosphate-buffered saline; “PEG” refers to polyethylene glycol; “PVDF” refers to polyvinylidene fluoride; “RP” refers to reverse phase; “RPM” refers to revolutions per minute; “siRNA” refers to small interfering ribonucleic acid; “TEA” refers to triethylamine; “THF” refers to tetrahydrofuran; “TLC” refers to thin line chromatography; “TMP” refers to 2,2,6,6-tetramethylpiperidine; “UPLC” refers to ultra-performance liquid chromatography; and “UV” refers to ultraviolet.

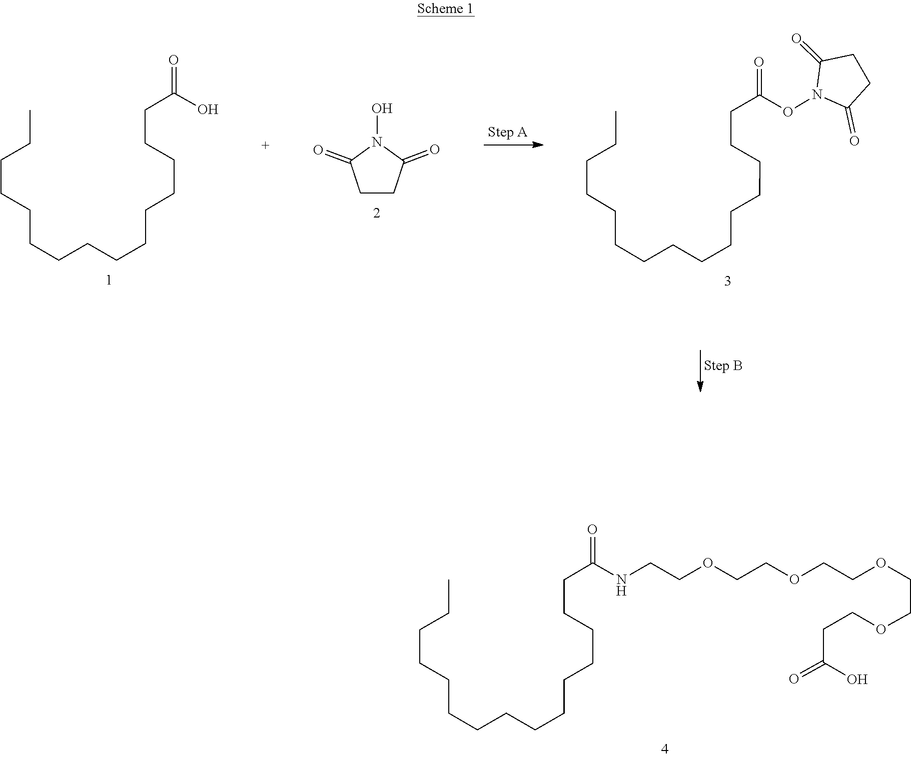 [SMILES: CCCCCCCCCCCCCCCC(=O)O ; O=C1CCC(=O)N1O ; CCCCCCCCCCCCCCCC(=O)ON1C(=O)CCC1=O ; CCCCCCCCCCCCCCCC(=O)NCCOCCOCCOCCOCCC(=O)O]

Scheme 1, step A depicts the coupling of compounds (1) and (2) using an appropriate base such as DMAP in a suitable solvent such as DCM to give compound (3). Step B shows the coupling of compound (3) with 1-amino-3,6,9,12-tetraoxapentadecan-15-oic acid in the presence of a base such as potassium carbonate and in a solvent system such as water and THF to give compound (4).

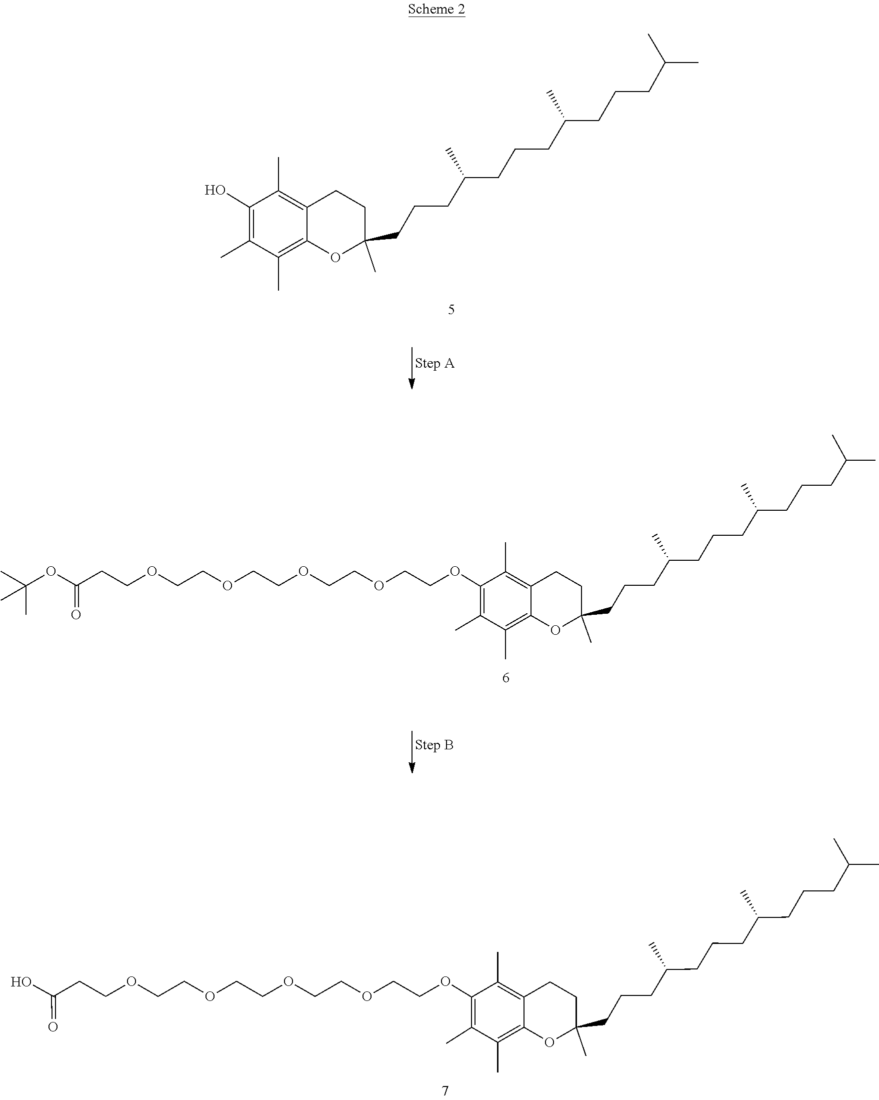 [SMILES: Cc1c(C)c2c(c(C)c1O)CC[C@@](C)(CCC[C@H](C)CCC[C@H](C)CCCC(C)C)O2 ; Cc1c(C)c2c(c(C)c1OCCOCCOCCOCCOCCC(=O)OC(C)(C)C)CC[C@@](C)(CCC[C@H](C)CCC[C@H](C)CCCC(C)C)O2 ; Cc1c(C)c2c(c(C)c1OCCOCCOCCOCCOCCC(=O)O)CC[C@@](C)(CCC[C@H](C)CCC[C@H](C)CCCC(C)C)O2]

Scheme 2, step A depicts a Mitsunobu reaction between compound (5) and tert-butyl 1-hydroxy-3,6,9,12-tetraoxapentadecan-15-oate using triphenyl phosphene and diisopropyl azodicarboxylate in a solvent such as THF to give compound (6). Step B shows the acidic deprotection of compound (6) using an acid such as HCl in a solvent such as 1,4-dioxane to give compound (7).

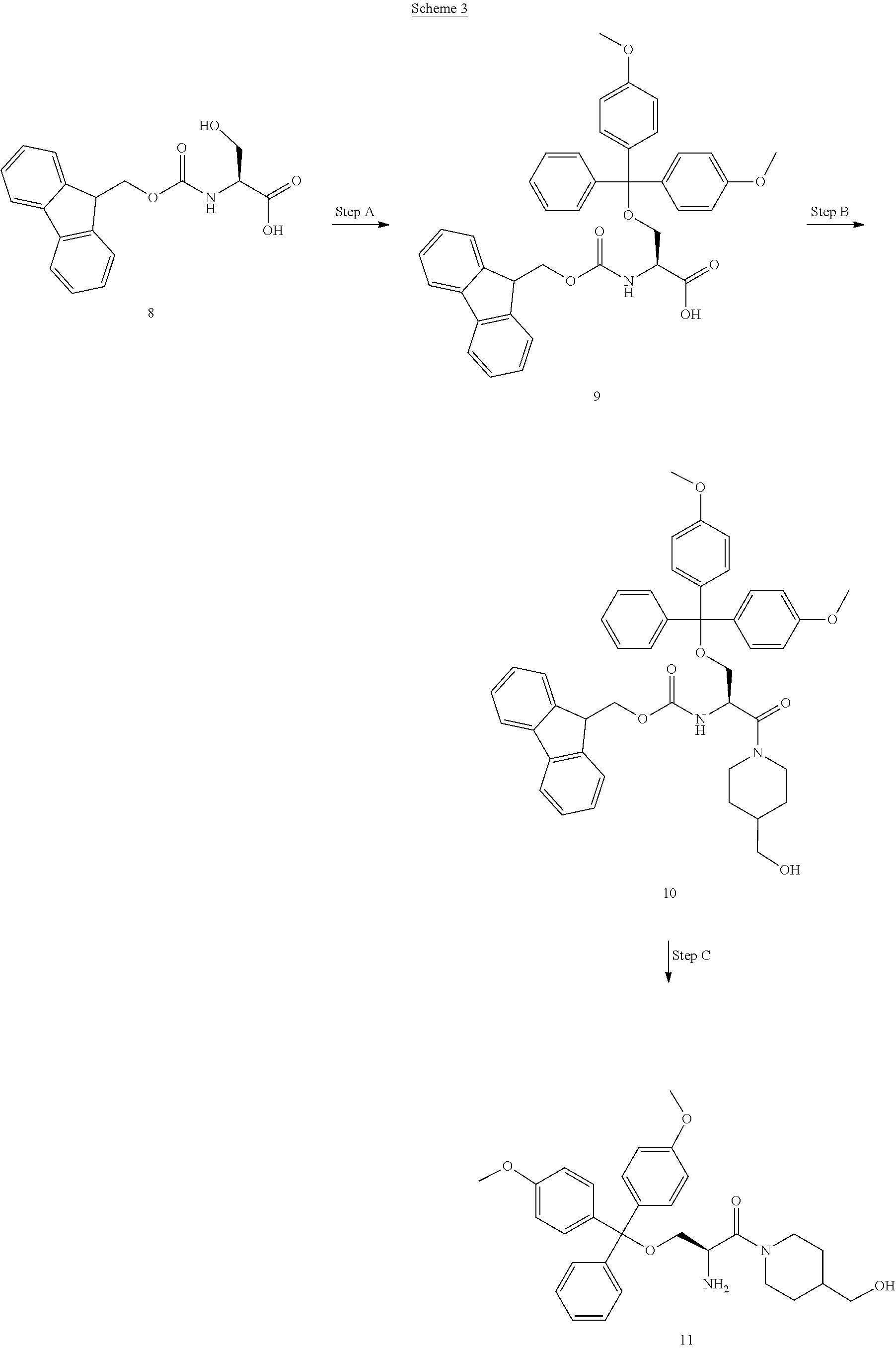 [SMILES: O=C(N[C@@H](CO)C(=O)O)OCC1c2ccccc2-c2ccccc21 ; COc1ccc(C(OC[C@H](NC(=O)OCC2c3ccccc3-c3ccccc32)C(=O)O)(c2ccccc2)c2ccc(OC)cc2)cc1 ; COc1ccc(C(OC[C@H](NC(=O)OCC2c3ccccc3-c3ccccc32)C(=O)N2CCC(CO)CC2)(c2ccccc2)c2ccc(OC)cc2)cc1 ; COc1ccc(C(OC[C@H](N)C(=O)N2CCC(CO)CC2)(c2ccccc2)c2ccc(OC)cc2)cc1]

Scheme 3, step A depicts the protection of compound (8) using DMTCl with a suitable base such as DIEA in a solvent such as DCM to give compound (9). Step B shows an amide coupling between compound (9) and piperidin-4-yl methanol using HBTU and HOBt with TMP in a solvent such as DCM to give compound (10). The deprotection of compound (10) with 20% piperidine in DMF to give compound (11) is shown in step C.

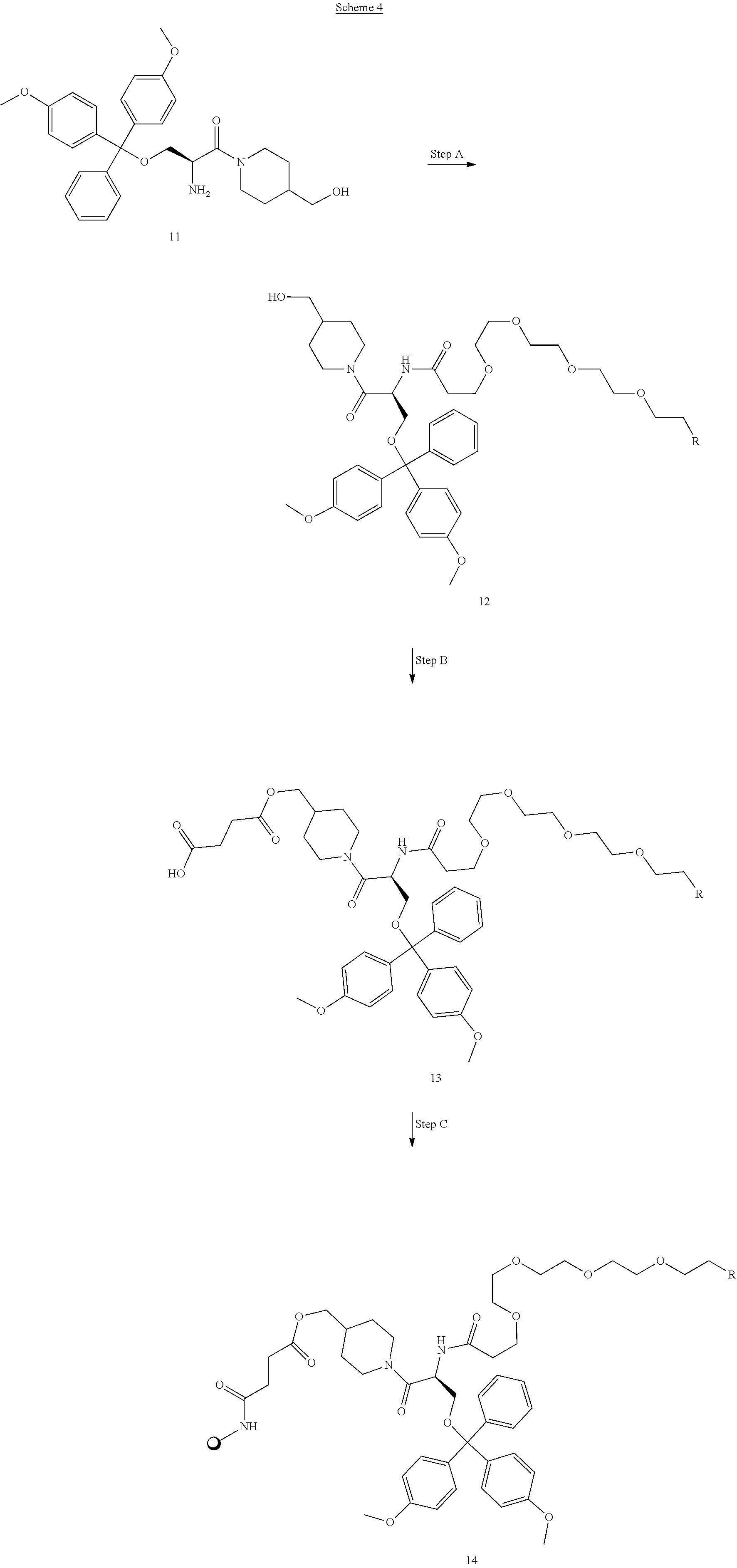

Scheme 4, step A depicts an amide coupling between compound (11) and either compound (4) or compound (7) using standard coupling reagents such as HBTU and HOBt with a base such as DIEA in a solvent such as DMF to give compound (12). One skilled in the art will recognize the variety of conditions which could be used to perform this amide coupling. Step B shows the coupling of compound (12) to succinic anhydride using a base such as TEA with catalytic DMAP in a solvent such as DCM to give compound (13). Step C shows the amide coupling of compound (13) to amino LCAA CPG using HBTU with a base such as DIEA in a solvent such as ACN followed by a multistep work up to give compound (14).

### Preparation 1

### 2,5-Dioxopyrrolidin-1-yl palmitate

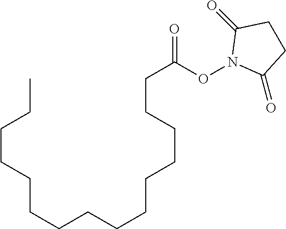 [SMILES: CCCCCCCCCCCCCCCC(=O)ON1C(=O)CCC1=O]

Added palmitic acid (2.00 g, 7.80 mmol) to a solution of 1-(3-dimethylaminopropyl)-3-ethylcarbodiimide hydrochloride (1.79 g, 9.36 mmol) and DMAP (0.19 g, 1.56 mmol) in DCM (31 mL). Stirred the mixture at ambient temperature for 5 minutes then added N-hydroxysuccinimide (0.99 g, 8.58 mmol) and stirred at ambient temperature for 18 hours. Concentrated in vacuo and purified the resulting crude material via silica gel flash chromatography eluting with a gradient of 0-80% EtOAc in hexanes to give the title compound as a white solid (2.65 g, 96%). 1H NMR (DMSO-d₆) δ 2.81 (s, 4H), 2.66 (t, 2H), 1.62 (m, 2H), 1.25 (br s, 24H), 0.87 (t, 3H).

### Preparation 2

### 3-[2-[2-[2-[2-(Hexadecanoylamino) ethoxy]ethoxy]ethoxy]ethoxy]propanoic acid

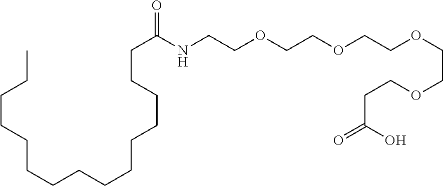 [SMILES: CCCCCCCCCCCCCCCC(=O)NCCOCCOCCOCCOCCC(=O)O]

Added 1-amino-3,6,9,12-tetraoxapentadecan-15-oic acid (0.14 g, 0.53 mmol) to a solution of potassium carbonate (0.14 g, 1.00 mmol) in THF (1 mL) and water (2 mL). Added 2,5-dioxopyrrolidin-1-yl palmitate (0.18 g, 0.51 mmol) and stirred the reaction at ambient temperature for 18 hours. Quenched the reaction with water (30 mL) and adjusted the pH to ˜3 with IN aqueous HCl. A precipitate formed and was collected by vacuum filtration to give the title compound as a white solid (0.19 g, 74%). ES/MS m z 504 (M+H).

### Preparation 3

### tert-Butyl 3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propanoate

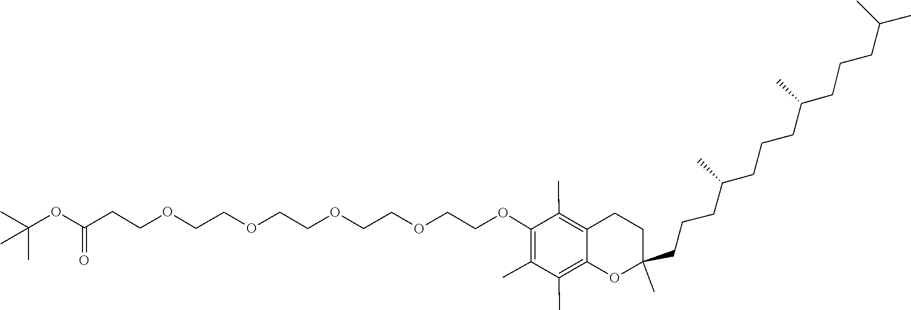 [SMILES: Cc1c(C)c2c(c(C)c1OCCOCCOCCOCCOCCC(=O)OC(C)(C)C)CC[C@@](C)(CCC[C@H](C)CCC[C@H](C)CCCC(C)C)O2]

Combined (2R)-2,5,7,8-tetramethyl-2-[(4R,8R)-4,8,12-trimethyltridecyl]chroman-6-ol (3.00 g, 6.90 mmol), tert-butyl 1-hydroxy-3,6,9,12-tetraoxapentadecan-15-oate (2.50 g, 7.60 mmol), and triphenylphosphine (2.00 g, 7.60 mmol) in THF (28.0 mL) and added diisopropyl azodicarboxylate (1.50 mL, 7.60 mmol) dropwise over 5 minutes. Heated the mixture at 60° C. for 16 hours. Cooled the mixture to ambient temperature, added silica gel, and concentrated in vacuo to give an off-white solid. Purified the mixture via silica gel flash chromatography, eluting with 0-40% EtOAc/hexanes, to give the title compound as an oil (3.33 g, 66%). 1H NMR (CDCl₃): 3.84 (s, 4H), 3.77-3.71 (m, 13H), 2.59 (t, J=6.8 Hz, 2H), 2.52 (t, J=6.6 Hz, 2H), 2.20-2.20 (m, 3H), 2.15-2.12 (m, 3H), 2.10 (s, 3H), 1.87-1.73 (m, 2H), 1.58-1.51 (m, 4H), 1.47 (s, 9H), 1.35-1.27 (m, 21H), 0.90-0.86 (m, 12H).

### Preparation 4

### 3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propanoic acid

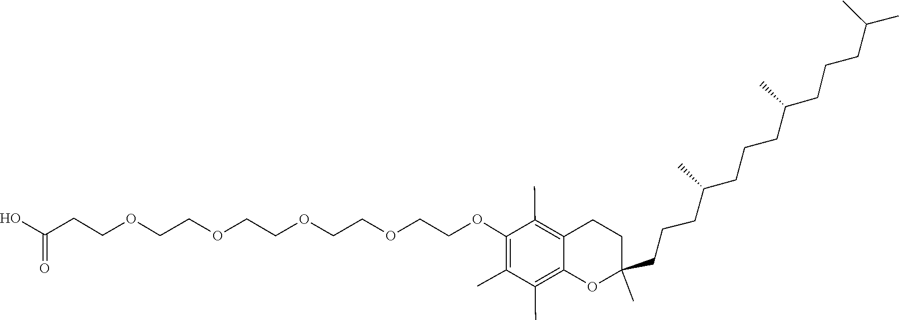 [SMILES: Cc1c(C)c2c(c(C)c1OCCOCCOCCOCCOCCC(=O)O)CC[C@@](C)(CCC[C@H](C)CCC[C@H](C)CCCC(C)C)O2]

Dissolved tert-butyl 3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propanoate (3.33 g, 4.53 mmol) in 4M HCl in dioxane (22.6 mL, 90.6 mmol) and stirred for 16 hours at ambient temperature. Removed the solvent under reduced pressure to give the title compound as an off-white solid (3.08 g, 100%). ES/MS m/z 678.0 (M−H).

### Preparation 5

### (2S)-3-[Bis (4-methoxyphenyl)-phenyl-methoxy]-2-(9H-fluoren-9ylmethoxycarbonylamino) propanoic acid

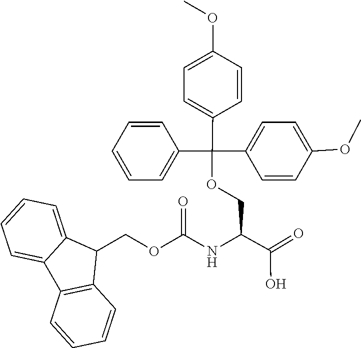 [SMILES: COc1ccc(C(OC[C@H](NC(=O)OCC2c3ccccc3-c3ccccc32)C(=O)O)(c2ccccc2)c2ccc(OC)cc2)cc1]

Added DIEA (64 mL, 0.366 mol) to a stirring solution of (2S)-2-(9H-fluoren-9-ylmethoxycarbonylamino)-3-hydroxy-propanoic acid (40 g, 0.122 mol) in dry DCM (400 mL) at 0° C. under an inert atmosphere. To this mixture, slowly added a solution of DMTCl (49.6 g, 0.146 mol) in DCM (200 mL). Allowed to warm to ambient temperature and stirred for 16 hours. After this time, diluted the reaction mixture with water and extracted with DCM. Dried organics over anhydrous sodium sulphate, filtered, and concentrated in vacuo. Washed the crude residue with 10% EtOAc/hexane and dried under vacuum to give the crude title compound as a pale brown solid (62 g, crude). TLC: 5% MeOH/CH₂Cl₂ (Rf. 0.5) UV, 254 nM.

### Preparation 6

### 9H-Fluoren-9-ylmethyl N-[(1S)-1-[bis (4-methoxyphenyl)-phenyl-methoxy]methyl]-2-[4-(hydroxymethyl)-1-piperidyl]-2-oxo-ethyl]carbamate

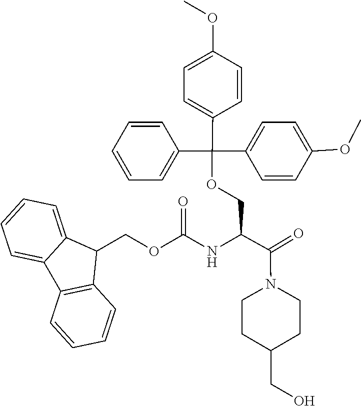 [SMILES: COc1ccc(C(OC[C@H](NC(=O)OCC2c3ccccc3-c3ccccc32)C(=O)N2CCC(CO)CC2)(c2ccccc2)c2ccc(OC)cc2)cc1]

Slowly added HBTU (78.3 g, 0.206 mol), HOBt (27.9 g, 0.206 mol), and piperidin-4-yl methanol (15.4 g, 0.134 mol) followed by TMP (15 mL, 0.113 mol) to a stirring solution of (2S)-3-[bis (4-methoxyphenyl)-phenyl-methoxy]-2-(9H-fluoren-9-ylmethoxycarbonylamino) propanoic acid (62 g, 0.103 mol) in DCM (750 mL) at 0° C. under inert atmosphere. Allowed the resulting reaction mixture to reach ambient temperature and stirred for 4 hours. After this time, diluted the mixture with water and extracted with DCM. Dried the organics over anhydrous sodium sulphate, filtered, and concentrated in vacuo. Purified the resulting residue via silica gel flash chromatography eluting with 20-40% EtOAc/hexane and 1% MeOH/DCM to give the title compound (40 g, 52% over two steps). 1H NMR (DMSO-d₆) δ 7.88 (br d, J=7.5 Hz, 2H), 7.79-7.59 (m, 3H), 7.45-7.12 (m, 13H), 6.92-6.76 (m, 4H), 4.79-4.44 (m, 2H), 4.32 (br d, J=11.4 Hz, 2H), 4.20 (br s, 2H), 3.71 (s, 6H), 3.21 (br s, 4H), 2.99-2.79 (m, 1H), 2.69 (br s, 2H), 1.81-1.43 (m, 3H), 1.08-0.73 (m, 2H).

### Preparation 7

### (2S)-2-Amino-3-[bis (4-methoxyphenyl)-phenyl-methoxy]-1-[4-(hydroxymethyl)-1-piperidyl]propan-1-one

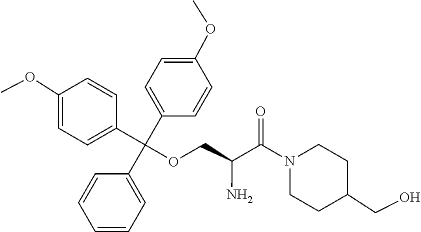 [SMILES: COc1ccc(C(OC[C@H](N)C(=O)N2CCC(CO)CC2)(c2ccccc2)c2ccc(OC)cc2)cc1]

Slowly added a solution of 20% piperidine in DMF (400 mL) to 9H-fluoren-9-ylmethyl N-[(1S)-1-[[bis (4-methoxyphenyl)-phenyl-methoxy]methyl]-2-[4-(hydroxymethyl)-1-piperidyl]-2-oxo-ethyl]carbamate (40 g, 0.055 mol) at 0° C. under inert atmosphere. Allowed the mixture to warm to ambient temperature and stirred for 1 hour. After this time, diluted the mixture with water and extracted with EtOAc. Dried organics over anhydrous sodium sulphate, filtered, and concentrated in vacuo. Purified the resulting residue via silica gel flash chromatography eluting with 1-8% MeOH/DCM to give the title compound as an off-white solid (13 g, 47%). ES/MS m/z 1009.5 (2M+H).

### Preparation 8

### N-[2-[2-[2-[2-[3-[(1S)-1-[Bis (4-methoxyphenyl)-phenyl-methoxy]methyl]-2-[4-(hydroxymethyl)-1-piperidyl]-2-oxo-ethyl]amino]-3-oxo-propoxy]ethoxy]ethoxy]ethoxy]ethyl]hexadecanamide

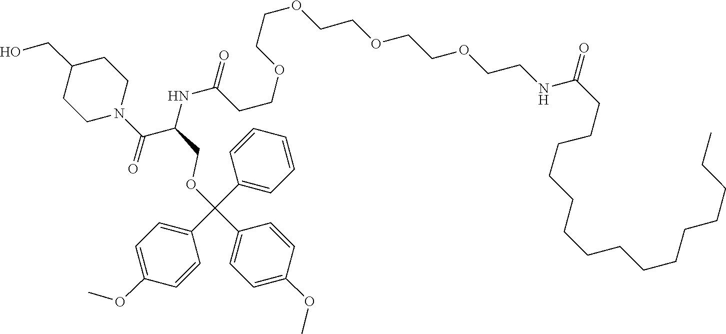 [SMILES: CCCCCCCCCCCCCCCC(=O)NCCOCCOCCOCCOCCC(=O)N[C@@H](COC(c1ccccc1)(c1ccc(OC)cc1)c1ccc(OC)cc1)C(=O)N1CCC(CO)CC1]

Combined 3-[2-[2-[2-[2-(hexadecanoylamino) ethoxy]ethoxy]ethoxy]ethoxy]propanoic acid (496 mg, 0.984 mmol), HOBt (146 mg, 1.08 mmol), HBTU (410 mg, 1.08 mmol), and DIEA (1.03 mL, 5.90 mmol) in DMF (9.84 mL) and stirred at ambient temperature for 10 minutes. Added (2S)-2-amino-3-[bis (4-methoxyphenyl)-phenyl-methoxy]-1-[4-(hydroxymethyl)-1-piperidyl]propan-1-one (546 mg, 1.08 mmol) to the mixture and stirred at ambient temperature for 16 hours. Partitioned the mixture between EtOAc and saturated aqueous sodium chloride solution. Separated the layers and washed the organics with saturated aqueous sodium chloride solution. Dried the organics over sodium sulfate, filtered, and concentrated in vacuo. Purified the resulting residue by silica gel flash chromatography eluting with 0-10% MeOH/DCM to give the title compound as an oil (327 mg, 34%). 1H NMR (DMSO-d₆) 8.21 (d, J=8.5 Hz, 1H), 7.80 (t, J=5.6 Hz, 1H), 7.37-7.28 (m, 4H), 7.23-7.20 (m, 5H), 6.88 (d, J=8.3 Hz, 4H), 5.06-5.02 (m, 1H), 4.51-4.49 (m, 1H), 4.45-4.40 (m, 1H), 3.97-3.93 (m, 1H), 3.74 (s, 5H), 3.63-3.56 (m, 2H), 3.49-3.48 (m, 4H), 3.47-3.45 (m, 7H), 3.40-3.35 (m, 2H), 3.30 (s, 1H), 3.23-3.13 (m, 7H), 2.41-2.33 (m, 2H), 2.04 (t, J=7.4 Hz, 2H), 1.74-1.69 (m, 3H), 1.51-1.44 (m, 2H), 1.26-1.24 (m, 24H), 1.00-0.97 (m, 1H), 0.88-0.82 (m, 5H).

### Preparation 9

### 4-[1-[(2S)-3-[Bis (4-methoxyphenyl)-phenyl-methoxy]-2-[3-[2-[2-[2-[2-(hexadecanoylamino) ethoxy]ethoxy]ethoxy]ethoxy]propanoylamino]propanoyl]-4-piperidyl]methoxy]-4-oxo-butanoic acid

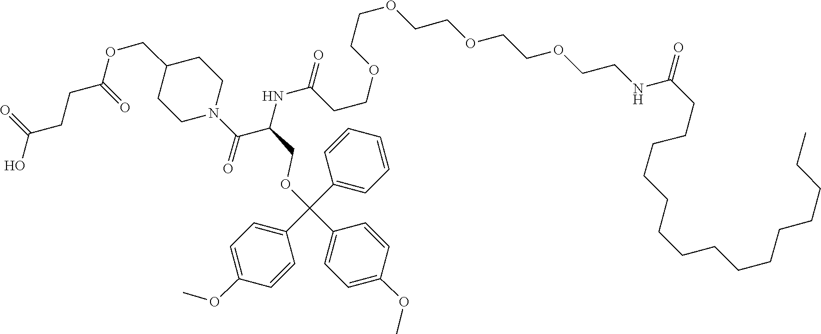 [SMILES: CCCCCCCCCCCCCCCC(=O)NCCOCCOCCOCCOCCC(=O)N[C@@H](COC(c1ccccc1)(c1ccc(OC)cc1)c1ccc(OC)cc1)C(=O)N1CCC(COC(=O)CCC(=O)O)CC1]

Combined N-[2-[2-[2-[2-[3-[(1S)-1-[[bis (4-methoxyphenyl)-phenyl-methoxy]methyl]-2-[4-(hydroxymethyl)-1-piperidyl]-2-oxo-ethyl]amino]-3-oxo-propoxy]ethoxy]ethoxy]ethoxy]ethyl]hexadecanamide (320 mg, 0.323 mmol), DMAP (120 mg, 0.969 mmol), TEA (225 μL, 1.62 mmol), and succinic anhydride (64.7 mg, 0.646 mmol) in DCM (6.46 mL) and stirred the mixture for 16 hours at ambient temperature. Purified the mixture directly via silica gel flash chromatography, eluting with 0% to 40% MeOH/DCM, to give the title compound as a colorless oil (279 mg, 79%). 1H NMR (DMSO-d₆) 12.65-12.64 (m, 1H), 8.24-8.19 (m, 1H), 7.80 (t, J=5.6 Hz, 1H), 7.37-7.28 (m, 4H), 7.24-7.20 (m, 5H), 6.88 (d, J=8.6 Hz, 4H), 5.05-5.01 (m, 1H), 4.44-4.40 (m, 1H), 3.97-3.95 (m, 3H), 3.74 (s, 6H), 3.61-3.56 (m, 2H), 3.49-3.45 (m, 11H), 3.38 (t, J=5.9 Hz, 3H), 3.22-3.14 (m, 6H), 2.48-2.31 (m, 7H), 2.04 (t, J=7.4 Hz, 2H), 1.90-1.87 (m, 5H), 1.24 (s, 23H), 0.98-0.96 (m, 1H), 0.87-0.82 (m, 4H).

### Preparation 10

### 4-[1-[(2S)-3-[Bis (4-methoxyphenyl)-phenyl-methoxy]-2-[3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propanoylamino]propanoyl]-4-piperidyl]methoxy]-4-oxo-butanoic acid

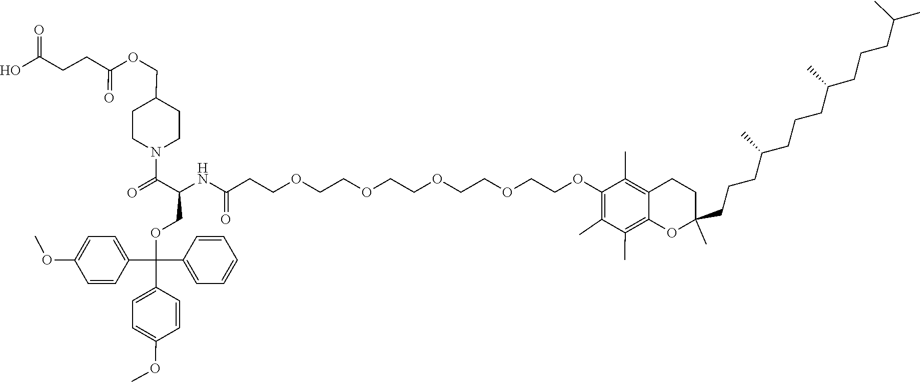 [SMILES: COc1ccc(C(OC[C@H](NC(=O)CCOCCOCCOCCOCCOc2c(C)c(C)c3c(c2C)CC[C@@](C)(CCC[C@H](C)CCC[C@H](C)CCCC(C)C)O3)C(=O)N2CCC(COC(=O)CCC(=O)O)CC2)(c2ccccc2)c2ccc(OC)cc2)cc1]

Combined 3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propanoic acid (1.20 g, 1.80 mmol,), HOBt (260 mg, 1.90 mmol), HBTU (740 mg, 1.90 mmol), and DIEA (1.80 mL, 11.0 mmol) in DMF (18.0 mL) and stirred at ambient temperature for 10 minutes. Added (2S)-2-amino-3-[bis (4-methoxyphenyl)-phenyl-methoxy]-1-[4-(hydroxymethyl)-1-piperidyl]propan-1-one (980 mg, 1.90 mmol) to the mixture and stirred at ambient temperature for 16 hours. Partitioned the mixture between EtOAc and saturated aqueous sodium chloride solution. Separated the layers and washed the organics with saturated aqueous sodium chloride solution. Dried the organic layer over sodium sulfate, filtered, and concentrated in vacuo. Purified the resulting residue by silica gel flash chromatography, eluting with 0-10% MeOH/DCM, to give N-[(1S)-1-[[bis (4-methoxyphenyl)-phenyl-methoxy]methyl]-2-[4-(hydroxymethyl)-1-piperidyl]-2-oxo-ethyl]-3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propenamide as a yellow oil.

Combined N-[(1S)-1-[bis (4-methoxyphenyl)-phenyl-methoxy]methyl]-2-[4-(hydroxymethyl)-1-piperidyl]-2-oxo-ethyl]-3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propenamide (1.45 g, 1.24 mmol), DMAP (456 mg, 3.73 mmol), TEA (867 μL, 6.22 mmol), and succinic anhydride (249 mg, 2.49 mmol) in DCM (24.9 mL) and stirred for 16 hours at ambient temperature. Concentrated in vacuo and purified the resulting residue via silica gel flash chromatography, eluting with 0-40% MeOH/DCM, to give the title compound as an oil (1.36 g, 60%). ES/MS m/z 1264.4 (M−H).

### Preparation 11

### [4-[1-[(2S)-3-[Bis (4-methoxyphenyl)-phenyl-methoxy]-2-[3-[2-[2-[2-[2-(hexadecanoylamino) ethoxy]ethoxy]ethoxy]ethoxy]propanoylamino]propanoyl]-4-piperidyl]methoxy]-4-oxo-butanoyl]amino] on CPG

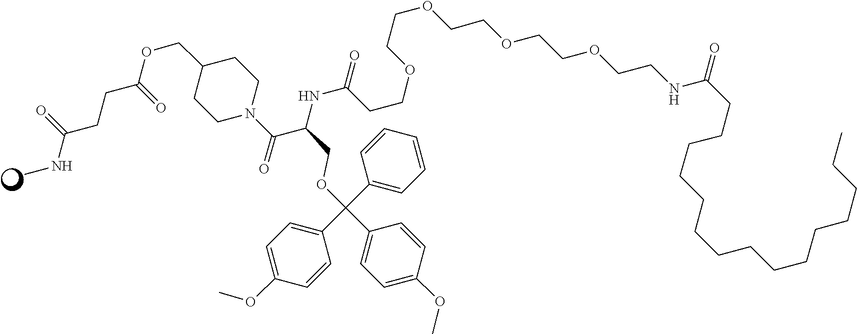 [SMILES: CCCCCCCCCCCCCCCC(=O)NCCOCCOCCOCCOCCC(=O)N[C@@H](COC(c1ccccc1)(c1ccc(OC)cc1)c1ccc(OC)cc1)C(=O)N1CCC(COC(=O)CCC(=O)NC)CC1]

Dissolved 4-[1-[(2S)-3-[bis (4-methoxyphenyl)-phenyl-methoxy]-2-[3-[2-[2-[2-[2-(hexadecanoylamino) ethoxy]ethoxy]ethoxy]ethoxy]propanoylamino]propanoyl]-4-piperidyl]methoxy]-4-oxo-butanoic acid (270 mg, 0.248 mmol) in ACN (12.5 mL) and transferred the solution to a fritted glass dropping funnel. Added DIEA (150 μL, 0.860 mmol) and HBTU (190 mg, 0.500 mmol) to the solution and shook the mixture at ambient temperature for 10 minutes. Added native amino LCAA CPG 500 Å (1.92 g, 129 μmol/g,) to the solution and shook the mixture at 500 RPM for 16 hours at ambient temperature. Drained the CPG and dried under nitrogen for 5 minutes. Washed the CPG with DCM (50 mL), 10% MeOH/DCM (50 mL), and then diethyl ether (50 mL). Dried the CPG for 30 minutes under nitrogen and then resuspended in pyridine (15 mL). Added acetic anhydride (3.30 mL, 35.0 mmol) and TEA (0.50 mL) and shook the mixture at 500 RPM for 2 hours at ambient temperature. Drained the CPG and dried for 5 minutes under nitrogen. Washed the CPG with DCM (50 mL), 10% MeOH/DCM (50 mL), and then diethyl ether (50 mL). Dried the CPG for 45 minutes under nitrogen and determined the ligand loading at 505 nm to give the title compound (1.92 g, 75.5 μmol/g).

### Preparation 12

### [4-[1-[(2S)-3-[Bis (4-methoxyphenyl)-phenyl-methoxy]-2-[3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propanoylamino]propanoyl]-4-piperidyl]methoxy]-4-oxo-butanoyl]amino] on CPG

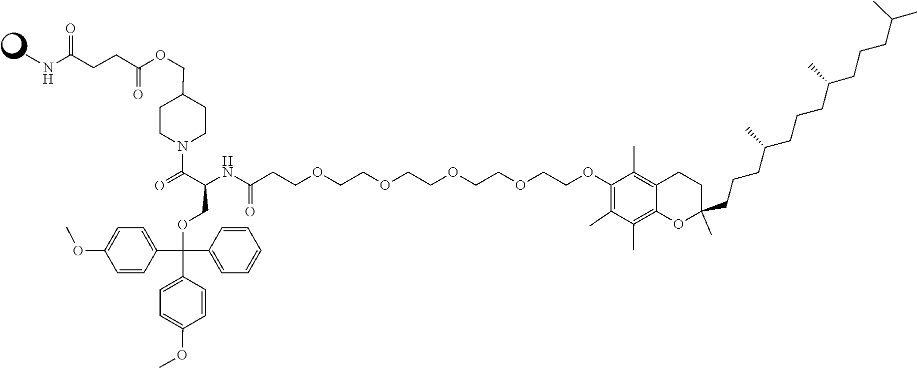 [SMILES: CNC(=O)CCC(=O)OCC1CCN(C(=O)[C@H](COC(c2ccccc2)(c2ccc(OC)cc2)c2ccc(OC)cc2)NC(=O)CCOCCOCCOCCOCCOc2c(C)c(C)c3c(c2C)CC[C@@](C)(CCC[C@H](C)CCC[C@H](C)CCCC(C)C)O3)CC1]

Prepared the title compound from 4-[1-[(2S)-3-[bis (4-methoxyphenyl)-phenyl-methoxy]-2-[3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propanoylamino]propanoyl]-4-piperidyl]methoxy]-4-oxo-butanoic acid in a manner essentially analogous to Preparation 11. Determined the ligand loading at 505 nm to give the title compound (4.01 g, 66.9 μmol/g).

### Preparation 13

### 3-[[(2R,3R,4R,5R)-2-[Bis (4-methoxyphenyl)-phenyl-methoxy]methyl]-4-hexadecoxy-5-(2-hydroxy-4-oxo-pyrimidin-1-yl)THF-3-yl]oxy-(diisopropylamino)phosphanyl]oxypropanenitrile

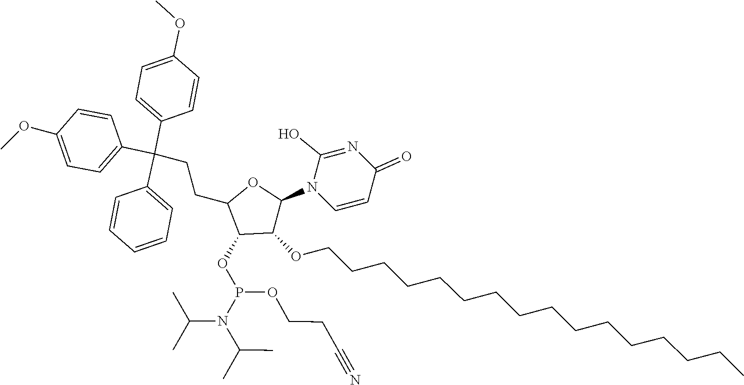 [SMILES: CCCCCCCCCCCCCCCCO[C@@H]1[C@H](OP(OCCC#N)N(C(C)C)C(C)C)C(CCC(c2ccccc2)(c2ccc(OC)cc2)c2ccc(OC)cc2)O[C@H]1n1ccc(=O)nc1O]

Prepared the title compound according to the protocols described in WO2019217459. ¹H NMR (CD₃CN) δ 7.86-7.73 (m, 1H), 7.51-7.43 (m, 2H), 7.40-7.23 (m, 7H), 6.95-6.87 (m, 4H), 5.90-5.84 (m, 1H), 5.29-5.21 (m, 1H), 4.54-4.40 (m, 1H), 4.21-4.13 (m, 1H), 4.10-3.56 (m, 13H), 3.50-3.34 (m, 2H), 2.75-2.62 (m, 1H), 2.55 (t, J=6.0 Hz, 1H), 1.66-1.51 (m, 2H), 1.40-1.14 (m, 35H), 1.08 (d, J=6.8 Hz, 3H), 0.91 (t, J=6.8 Hz, 3H). ³¹P NMR (CD₃CN): 149.6, 149.2.

### Preparation 14

### N-[9-[(2R,3R,4R,5R)-5-[Bis (4-methoxyphenyl)-phenyl-methoxy]methyl]-4-[2-cyanoethoxy-(diisopropylamino)phosphanyl]oxy-3-hexadecoxy-tetrahydrofuran-2-yl]purin-6-yl]benzamide

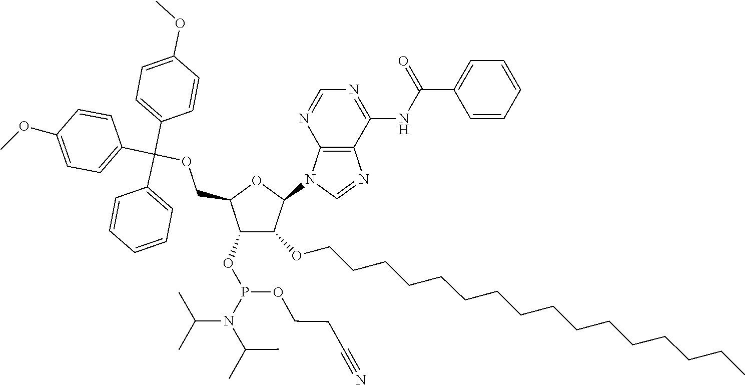 [SMILES: CCCCCCCCCCCCCCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1n1cnc2c(NC(=O)c3ccccc3)ncnc21]

Prepared the title compound according to the protocols described in WO2019217459. ¹H-NMR (CD₃CN) δ 9.37 (s, 1H), 8.57 (d, J=9.4 Hz, 1H), 8.27 (d, J=10.3 Hz, 1H), 7.99 (d, J=7.6 Hz, 2H), 7.61 (d, J=7.4 Hz, 1H), 7.52 (t, J=7.6 Hz, 2H), 7.42 (t, J=7.3 Hz, 2H), 7.34-7.16 (m, 7H), 6.85-6.77 (m, 4H), 6.11 (dd, J=5.0, 2.5 Hz, 1H), 4.80 (m, 1H), 4.69 (m, 1H), 4.32 (m, 1H), 3.97-3.78 (m, 1H), 3.74 (d, J=3.1 Hz, 7H), 3.64 (m, 4H), 3.56-3.40 (m, 2H), 3.33 (m, 1H), 2.73-2.59 (m, 1H), 2.50 (t, J=6.0 Hz, 1H), 1.52-1.45 (m, 2H), 1.33-1.12 (m, 37H), 1.09 (d, J=6.8 Hz, 3H), 0.87 (t, J=6.8 Hz, 3H). ³¹P NMR (CD₃CN) δ 151.19, 150.78.

### Preparation 15

### N-[1-[(2R,3R,4R,5R)-5-[[Bis (4-methoxyphenyl)-phenyl-methoxy]methyl]-4-[2-cyanoethoxy-(diisopropylamino)phosphanyl]oxy-3-hexadecoxy-tetrahydrofuran-2-yl]-2-oxo-pyrimidin-4-yl]acetamide

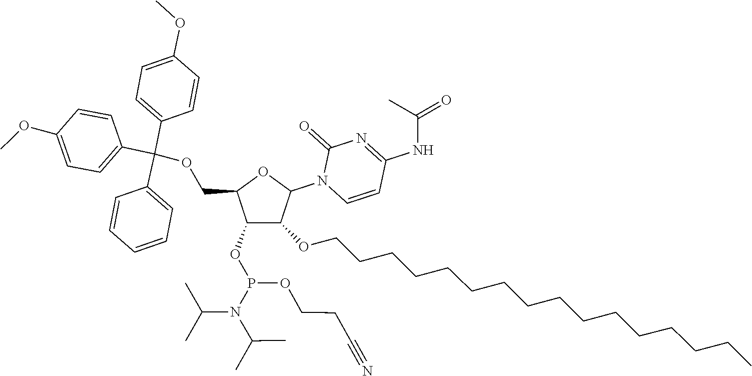 [SMILES: CCCCCCCCCCCCCCCCO[C@H]1C(n2ccc(NC(C)=O)nc2=O)O[C@H](COC(c2ccccc2)(c2ccc(OC)cc2)c2ccc(OC)cc2)[C@H]1OP(OCCC#N)N(C(C)C)C(C)C]

Prepared the title compound according to the protocols described in WO2019217459. ¹H-NMR (CD₃CN) δ 9.15 (s, 1H), 8.46 (dd, J=7.5 Hz, 1H), 7.95 (d, J=7.6 Hz, 2H), 7.63 (t, J=7.5 Hz, 1H), 7.57-7.41 (m, 5H), 7.41-7.31 (m, 6H), 7.28 (m, 1H), 7.04 (d, J=15.8 Hz, 1H), 6.90 (t, J=7.9 Hz, 4H), 5.90 (d, J=7.8 Hz, 1H), 4.51 (m, 1H), 4.20 (dd, J=10.6, 8.1 Hz, 1H), 4.04 (dd, J=31.3, 4.6 Hz, 1H), 3.91-3.81 (m, 2H), 3.79 (d, J=3.1 Hz, 6H), 3.74 (m, 2H), 3.69-3.41 (m, 6H), 2.67-2.59 (m, 1H), 2.54-2.48 (m, 1H), 1.58 (m, 2H), 1.36 (m, 2H), 1.25 (d, J=4.7 Hz, 26H), 1.21-1.09 (m, 10H), 1.04 (d, J=6.8 Hz, 3H), 0.87 (t, J=6.8 Hz, 3H). ³¹P NMR (CD₃CN) δ 151.10, 150.19.

### Preparation 16

### N-[9-[(2R,3R,4R,5R)-5-[[Bis (4-methoxyphenyl)-phenyl-methoxy]methyl]-4-[2-cyanoethoxy-(diisopropylamino)phosphanyl]oxy-3-hexadecoxy-tetrahydrofuran-2-yl]-6-oxo-1H-purin-2-yl]-2-methyl-propanamide

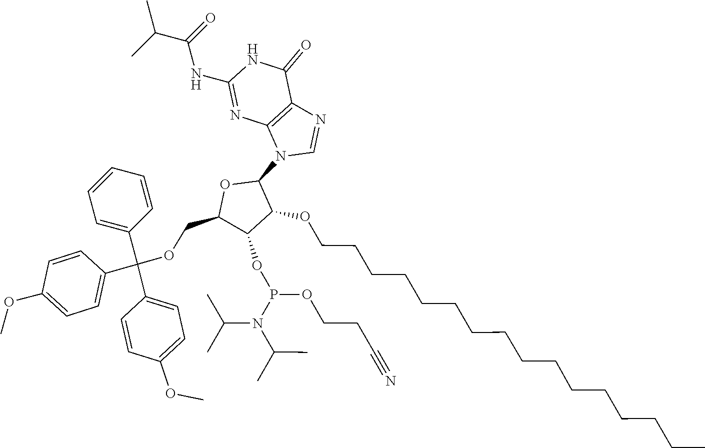 [SMILES: CCCCCCCCCCCCCCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1n1cnc2c(=O)[nH]c(NC(=O)C(C)C)nc21]

Prepared the title compound according to the protocols described in WO2019217459.

¹H-NMR (CDCl₃) δ 12.01-11.96 (m, 1H), 7.82-7.78 (m, 1H), 7.59-7.53 (m, 1H), 7.47-7.42 (m, 1H), 7.41-7.37 (m, 2H), 7.34-7.29 (m, 2H), 7.27-7.22 (m, 3H), 6.85-6.80 (m, 4H), 5.99-5.82 (m, 1H), 4.40-4.36 (m, 1H), 4.17-4.11 (m, 1H), 3.80-3.77 (m, 6H), 3.76-3.68 (m, 6H), 3.22-3.17 (m, 1H), 2.84-2.79 (m, 1H), 1.60-1.54 (m, 4H), 1.35-1.30 (m, 6H), 1.27 (s, 19H), 1.24-1.15 (m, 13H), 1.06-1.03 (m, 5H), 0.93-0.88 (m, 6H), 0.74-0.70 (m, 1H). ³¹P NMR (CDCl₃) δ 150.20, 149.92.

### Example 2. Synthesis of ATXN2 RNAi Agents

Single strands (sense and antisense) of the RNA duplexes were synthesized on solid support via a MerMade™ 12 (LGC Biosearch Technologies). The sequences of the sense and antisense strands were shown in Table 2. The oligonucleotides were synthesized via phosphoramidite chemistry at either 5, 10, 25 or 50 μmol scales.

For the sense strands, the types of solid supports were universal CPG: (3′-Piperidinol-PEG-Palmitate) and (3′-Piperidinol-PEG-Tocopherol) were synthesized in house (see Example 1) while the Universal UnyLinker (Chemgenes, Catalog No. AT273-27) and 3′Teg-Tocopherol (LGC Biosearch Technologies, Catalog No. BG7-1190) were purchased commercially. For all the antisense strands, commercially available standard support mA was utilized. Standard reagents were used in the oligo synthesis (Table 7), where 0.1M xanthane hydride in pyridine was used as the sulfurization reagent and 20% DEA in ACN was used as an auxiliary wash post synthesis. All monomers (Table 8) were made at 0.1M in ACN and contained a molecular sieves trap bag.

The oligonucleotides were cleaved and deprotected (C/D) at 45° C. for 20 hours. The sense strands were C/D from the CPG using ammonia hydroxide (28-30%, cold), whereas 3% DEA in ammonia hydroxide (28-30%, cold) was used for the antisense strands. C/D was determined complete by IP-RP LCMS when the resulting mass data confirmed the identity of sequence. Dependent on scale, the CPG was filtered via 0.45 um PVDF syringeless filter, 0.22 um PVDF Steriflip® vacuum filtration or 0.22 um PVDF Stericup® Quick release. The CPG was back washed/rinsed with either 30% ACN/RNAse free water or 30% EtOH/RNAse free water then filtered through the same filtering device and combined with the first filtrate. This was repeated twice. The material was then divided evenly into 50 mL falcon tubes to remove organics via Genevac™. After concentration, the crude oligonucleotides were diluted back to synthesized scale with RNAse free water and filtered either by 0.45 μm PVDF syringeless filter, 0.22 μm PVDF Steriflip® vacuum filtration or 0.22 μm PVDF Stericup® Quick release.

The crude oligonucleotides were purified via AKTA™ Pure purification system using either anion-exchange (AEX) or reverse phase (RP) a source 15Q-RP column. For AEX, an ES Industry Source™ 15Q column maintaining column temperature at 65° C. with MPA: 20 mM NaH₂PO₄, 15% ACN, pH 7.4 and MPB: 20 mM NaH₂PO₄, 1M NaBr, 15% ACN, pH 7.4. For RP, a Source™ 15Q-RP column with MPA: 50 mM NaOAc with 10% ACN and MPB: 50 mM NaOAc with 80% ACN. In all cases, fractions which contained a mass purity greater than 85% without impurities >5% where combined.

The purified oligonucleotides were desalted using 15 mL 3K MWCO centrifugal spin tubes at 3500×g for ˜30 min. The oligonucleotides were rinsed with RNAse free water until the eluent conductivity reached <100 usemi/cm. After desalting was complete, 2-3 mL of RNAse free water was added then aspirated 10×, the retainment was transferred to a 50 mL falcon tube, this was repeated until complete transfer of oligo by measuring concentration of compound on filter via nanodrop. The final oligonucleotide was then nano filtered 2× via 15 mL 100K MWCO centrifugal spin tubes at 3500×g for 2 min. The final desalted oligonucleotides were analyzed for concentration (nano drop at A260), characterized by IP-RP LCMS for mass purity and UPLC for UV-purity.

For the preparation of duplexes, equimolar amounts of sense and antisense strand were combined and heated at 65° C. for 10 minutes then slowly cooled to ambient temperature over 40 minutes. Integrity of the duplex was confirmed by UPLC analysis and characterized by LCMS using IP-RP. All duplexes were nano filtered then endotoxin levels measured via Charles River Endosafe® Cartridge Device to give the final compounds of conjugated RNAi (Table 9). For in vivo analysis, the appropriate amount of duplex was lyophilized then reconstituted in 1×PBS for rodent studies and a CSF for non-human primate studies.

TABLE 7
Oligonucleotide Synthesis Reagents
Reagents

Activator Solution (0.5M ETT in ACN)
Cap A (Acetic Anhydride, Pyridine in THF, 1:1:8)
Cap B (1-Methylimidazole in THF, 16:84)
Oxidation Solution (0.02M Iodine in THF/Pyridine/Water,
70:20:10)
Deblock Solution, 3% TCA in DCM (w/v)
Acetonitrile (Anhydrosolv, Water max. 10 ppm)
Xanthane Hydride (0.1M in Pyridine)
Diethylamine (20% in Acetonitrile)

TABLE 8
Phosphoramidites

| Phosphoramidite | Abbreviation | Supplier | Catalog # | CAS |
|---|---|---|---|---|
| DMT-2′-F-A(Bz)-CE | fA | Hongene | PD1-001 | 136834-22-5 |
| Phosphoamidite | | | | |
| DMT-2′-F-C(Ac)-CE | fC | Hongene | PD3-001 | 159414-99-0 |
| Phosphoamidite | | | | |
| DMT-2′-F-G(iBu)-CE | fG | Hongene | PD2-002 | 144089-97-4 |
| Phosphoamidite | | | | |
| DMT-2′-F-U-CE | fU | Hongene | PD5-001 | 146954-75-8 |
| Phosphoamidite | | | | |
| DMT-2′-O-Me-A(Bz)- | mA | Hongene | PR1-001 | 110782-31-5 |
| CE Phosphoamidite | | | | |
| DMT-2′-O-Me-G(iBu)- | mG | Hongene | PR2-002 | 150780-67-9 |
| CE Phosphoamidite | | | | |
| DMT-2′-O-Me-U-CE | mU | Hongene | PR5-001 | 110764-79-9 |
| Phosphoamidite | | | | |
| 5′bis(POM) vinyl | POM-VPmU | Hongene | PR5-032 | BVPMUP23B2A1 |
| phosphate-2′-Ome- | | | | |
| U3′CE | | | | |
| phosphoroamidite | | | | |
| Reverse Abasic | iAb | Chemgenes | ANP-1422 | 401813-16-9 |
| phosphoroamidite | | | | |
| Abasic | Ab | Chemgenes | ANP-7058 | 129821-76-7 |
| phosphoroamidite | | | | |
| Uhd | Uhd | Lilly | | |
| Chd | Chd | Lilly | | |
| Ahd | Ahd | Lilly | | |
| Ghd | Ghd | Lilly | | |

TABLE 9
Conjugated ATXN2 RNAi Agents

| Conjugated | | | MW | MW |
|---|---|---|---|---|
| RNAi | | | Cal. | Obs. |
| Agent No. | Strand | LDP* | (g/mol) | (g/mol) |
| 1 | S: SEQ ID NO: 31 | 8 | 7880.8 | 7880.8 |
| | AS: SEQ ID NO: 32 | | 7578.9 | 7579.2 |
| 2 | S: SEQ ID NO: 33 | 8 | 7706.6 | 7706.5 |
| | AS: SEQ ID NO: 34 | | 7767.1 | 7766.5 |
| 3 | S: SEQ ID NO: 35 | 8 | 7701.6 | 7702.4 |
| | AS: SEQ ID NO: 36 | | 7735.1 | 7734.5 |
| 4 | S: SEQ ID NO: 37 | 8 | 7904.8 | 7905.6 |
| | AS: SEQ ID NO: 38 | | 7539.8 | 7539.2 |
| 5 | S: SEQ ID NO: 39 | 8 | 7612.5 | 7613.7 |
| | AS: SEQ ID NO: 40 | | 7908.2 | 7908.7 |
| 6 | S: SEQ ID NO: 41 | 8 | 7629.5 | 7630.4 |
| | AS: SEQ ID NO: 42 | | 7891.2 | 7891.2 |
| 7 | S: SEQ ID NO: 43 | 8 | 7722.6 | 7723.1 |
| | AS: SEQ ID NO: 44 | | 7767.1 | 7766.5 |
| 8 | S: SEQ ID NO: 45 | 8 | 7620.5 | 7621.6 |
| | AS: SEQ ID NO: 46 | | 7870.2 | 7870.4 |
| 9 | S: SEQ ID NO: 47 | 8 | 7572.5 | 7572.7 |
| | AS: SEQ ID NO: 48 | | 7931.3 | 7931.3 |
| 10 | S: SEQ ID NO: 49 | 8 | 7651.5 | 7651.3 |
| | AS: SEQ ID NO: 50 | | 7907.2 | 7906.5 |
| 11 | S: SEQ ID NO: 51 | 8 | 7658.6 | 7658.5 |
| | AS: SEQ ID NO: 52 | | 7862.2 | 7862.5 |
| 12 | S: SEQ ID NO: 53 | 8 | 7884.7 | 7885.7 |
| | AS: SEQ ID NO: 54 | | 7552.0 | 7552.9 |
| 13 | S: SEQ ID NO: 55 | 8 | 7659.6 | 7659.3 |
| | AS: SEQ ID NO: 56 | | 7885.2 | 7885.8 |
| 14 | S: SEQ ID NO: 57 | 8 | 7594.6 | 7594.5 |
| | AS: SEQ ID NO: 58 | | 7940.4 | 7940.4 |
| 15 | S: SEQ ID NO: 59 | 8 | 7816.8 | 7816.9 |
| | AS: SEQ ID NO: 60 | | 7657.9 | 7658.2 |

“S” means the sense strand; “AS” means the antisense strand.
*LDP is linked to the 3′ end of the sense strand.

### Example 3. Characterization of ATXN2 RNAi Agents

Selected ATXN2 RNAi agents were tested in vitro for ATXN2 inhibition in cultured cells, including SH-SY5Y cells, mouse primary cortical neurons, and/or human induced pluripotent stem cells (hiPSC).

### Materials and Methods

SH-SY5Y Cell Culture and RNAi Treatment and Analysis: SH-SY5Y cells (ATCC CRL-2266) were derived from the SK-N-SH neuroblastoma cell line (Ross, R. A., et al., 1983. J Natl Cancer Inst 71, 741-747). The base medium was composed of a 1:1 mixture of ATCC-formulated Eagle's Minimum Essential Medium, (Cat No. 30-2003), and F12 Medium. The complete growth medium was supplemented with 10% fetal bovine serum, 1× amino acids, 1× sodium bicarbonate, and 1× penicillin-streptomycin (Gibco) and cells incubated at 37° C. in a humidified atmosphere of 5% CO₂. On Day One, SH-SY5Y cells were plated in 96 well fibronectin coated tissue culture plates and allowed to attach overnight. On Day Two, complete media was removed and replaced with RNAi agent in serum free media. Cells were incubated with RNAi agent for 72 hours before analysis of gene expression. Analysis of changes in gene expression in RNAi treated SH-SY5Y cells was measured using Cells-to-CT Kits following the manufacturer's protocol (ThermoFisher A35377). Predesigned gene expression assays (supplied as 20× mixtures) were selected from Applied Bio-systems (Foster City, CA, USA). The efficiencies of these assays (ThermoFisher Hs00240906_m1 ATXN2 and ThermoFisher Hs99999905_m1 GAPDH) were characterized with a dilution series of cDNA. RT-QPCR was performed in MicroAmp Optical 384-well reaction plates using QuantStudio 7 Flex system. The delta-delta CT method of normalizing to the housekeeping gene GAPDH was used to determine relative amounts of gene expression. GraphPad Prism v9.0 was used to determine IC50 with a four parameter logistic fit.

Mouse Primary Cortical Neuron (MCN) Culture and RNAi Treatment and Analysis: Mouse primary cortical neurons were isolated from wild type C57BL6 mouse embryos at E18. Cells were plated in poly-D-lysine coated 96-well plates at a density of 40k cells/well and cultured in NbActiv1 (BrainBits, LLC) containing 1% Antibiotic/Antimycotic (Corning) for 7 days at 37° C. in a tissue culture incubator in a humidified chamber with 5% CO₂. On Day 7, half of the medium was removed from each well and 2× concentration of RNAi in culture media with 2% FBS was added for treatment as CRC and incubated with cells for additional 7, 14 or 21 days. Half media change was done every 7 days with fresh culture media. At the end of RNAi treatment, RT-qPCR was performed to quantify ATXN2 mRNA levels using TaqMan Fast Advanced Cell-to-CT kit. Specifically, cells were lysed, cDNA was generated on Mastercycler X50a (Eppendorf), and qPCR was carried out on QuantStudio 7 Flex Real-Time PCR System (Applied Biosystems). Alpha-synuclein (ThermoFisher, Mm00447333_m1) gene expression levels were normalized by β-actin (ThermoFisher, Mm02619580_g1) using respective probes.

Human Induced Pluripotent Stem Cell-derived Neuron (hiPSC Neuron) Culture and RNAi Treatment and Analysis: Doxycycline-inducible Neurogenin2 (NGN2) human induced Pluripotent Stem Cells (hiPSC) were developed by Bioneer for Eli Lilly. The hiPSC were doxycycline-induced for three days (DIV3) to initiate neuronal differentiation and plated on 96-well PDL and laminin coated plates at 30k/well and grown in Neuronal Differentiation Media (NDM) consisting of DMEM/F12 (Life Technologies 11330-057), Neurobasal media (Gibco 15240062), antibiotics, supplements, growth factors and doxycycline in an incubator (37° C./5% CO₂). Cells were half-fed every seven days, and on DIV21, RNAi agent was serially diluted in NDM, and cells were treated with RNAi by aspirating 75 μL and adding 75 μL of 2×RNAi concentration for a final of 1×RNAi according to dilutions. Cells were half-fed every seven days after treatment by removing half of media and adding back fresh NDM. Cell lysates were harvested at DIV35 (14 days later) or DIV42 (21 days later) and RT-qPCR was performed using TaqMan Fast Advanced Cells-to-CT Kit (ThermoFisher, A35377) and to determine mRNA knock down using ATXN2 probe as the gene of interest (ThermoFisher, Hs00240907_m1) and ACTb probe as the housekeeping gene (ThermoFisher, Hs99999903_m1).

### Results

Table 10 summarizes the percentage knockdown of ATXN2 mRNA and IC50 of the SARM RNAi agents in human SH-SY5Y cells, mouse primary cortical neurons (MCN) and hiPSC neurons. The tested ATXN2 RNAi agents achieved ATXN2 mRNA knockdown in human SH-SY5Y cells.

TABLE 10
In vitro activities of selected ATXN2 RNAi agents

| | | | | | | hiPSC |
|---|---|---|---|---|---|---|
| | | | | | | Neurons, |
| | | SHSY5Y, 3 d | | MCN, 7 d | hiPSC | 21 d |
| | SHSY5Y, | % KD | | % KD | Neurons, | % KD |
| | 3 d | (knockdown) | | (knockdown) | 21 d | (knockdown) |
| | IC50 | of ATXN2 at | MCN, 7 d | of ATXN2 at | IC50 | of ATXN2 at |
| RNAi Agent | (nM) | 1 uM | IC50 (nM) | 1 uM | (nM) | 1 uM |
| Conjugated | 29.34 | 70.66 | 18.2 | 83 | 17.93 | 67.5 |
| RNAi Agent | | | | | | |
| NO. 1 | | | | | | |
| Conjugated | 32.26 | 67.74 | 11.4 | 72 | 62.43 | 75.8 |
| RNAi Agent | | | | | | |
| NO. 2 | | | | | | |
| Conjugated | 32.42 | 67.58 | 47.4 | 81 | 25.2 | 62.5 |
| RNAi Agent | | | | | | |
| NO. 3 | | | | | | |
| Conjugated | 34.53 | 65.47 | 167.9 | 76 | 50.68 | 72.6 |
| RNAi Agent | | | | | | |
| NO. 4 | | | | | | |
| Conjugated | 34.67 | 65.33 | 11.8 | 68 | 45.53 | 80.5 |
| RNAi Agent | | | | | | |
| NO. 5 | | | | | | |
| Conjugated | 34.80 | 65.20 | 15.2 | 63 | 51.68 | 68.2 |
| RNAi Agent | | | | | | |
| NO. 6 | | | | | | |
| Conjugated | 35.64 | 64.36 | 12.5 | 70 | 20.35 | 83.4 |
| RNAi Agent | | | | | | |
| NO. 7 | | | | | | |
| Conjugated | 37.20 | 62.80 | 18.6 | 61 | 137.9 | 51 |
| RNAi Agent | | | | | | |
| NO. 8 | | | | | | |
| Conjugated | 37.72 | 62.28 | 12.4 | 67 | 8.72 | 44.4 |
| RNAi Agent | | | | | | |
| NO. 9 | | | | | | |
| Conjugated | 37.76 | 62.24 | 17.3 | 71 | 80.86 | 66.7 |
| RNAi Agent | | | | | | |
| NO. 10 | | | | | | |
| Conjugated | 39.34 | 60.66 | 61.5 | 73 | 43.9 | 57.4 |
| RNAi Agent | | | | | | |
| NO. 11 | | | | | | |
| Conjugated | 39.59 | 60.41 | 14.3 | 78 | 97.62 | 56.7 |
| RNAi Agent | | | | | | |
| No. 12 | | | | | | |
| Conjugated | 39.59 | 60.41 | 126.4 | 70 | 35.61 | 63.1 |
| RNAi Agent | | | | | | |
| No. 13 | | | | | | |
| Conjugated | 39.60 | 60.40 | 108.2 | 7 | 1134 | 31.1 |
| RNAi Agent | | | | | | |
| No. 14 | | | | | | |
| Conjugated | 42.28 | 57.72 | 57.3 | 47 | 61.67 | 65.4 |
| RNAi Agent | | | | | | |
| No. 15 | | | | | | |

### Example 4. In Vivo Characterization of Selected RNAi Agents in Mice

The efficacy of the RNAi agents was studied in wildtype C56BL/6N mice. Six mice received intracerebroventricular (ICV) injection of 30 μg of the RNAi agent or PBS (phosphate buffered saline), and were sacrificed on Day 21 after the injection. Mouse ATXN2 mRNA expression in spinal cord and brain were measured and analyzed by quantitative PCR (qPCR). The results are shown in Table 11.

TABLE 11
The Percentage Knockdown (KD) of ATXN2 mRNA in Mice

| | | | Mouse ICV, 8 d | |
|---|---|---|---|---|
| Conjugated | Mouse ICV, 8 d | Mouse ICV, 8 d | % KD | Mouse ICV, 8 d |
| RNAi | % KD | % KD | Lumbar Spinal | % KD |
| Agent No. | Frontal Cortex | Brain stem | Cord | Cerebellum |
| 1 | 8 | 20 | 42 | 29 |
| 2 | 22 | 32 | 26 | 25 |
| 3 | 7 | 27 | 20 | 17 |
| 4 | 0 | 11 | 18 | 0 |
| 5 | 18 | 12 | 13 | 25 |
| 6 | 0 | 26 | 14 | 0 |
| 7 | 26 | 35 | 36 | 17 |
| 8 | 4 | 16 | 1 | 0 |
| 9 | 9 | 28 | 26 | 17 |
| 10 | 33 | 34 | 35 | 28 |
| 11 | 31 | 45 | 56 | 53 |
| 12 | 9 | 19 | 13 | 17 |
| 13 | 0 | 18 | 18 | 27 |
| 14 | 0 | 0 | 2 | 6 |
| 15 | 0 | 6 | 16 | 18 |

1. An ATXN2 RNAi agent comprising a sense strand and an antisense strand,
wherein the sense strand and the antisense strand form a duplex,
wherein the sense strand and the antisense strand comprise a pair of nucleic acid sequences selected from the group consisting of:
(a) the sense strand comprises a first nucleic acid sequence having at least 95% sequence identity to SEQ ID NO: 1, and the antisense strand comprises a second nucleic acid sequence having at least 95% sequence identity to SEQ ID NO: 2;
(b) the sense strand comprises a first nucleic acid sequence having at least 95% sequence identity to SEQ ID NO: 3, and the antisense strand comprises a second nucleic acid sequence having at least 95% sequence identity to SEQ ID NO: 4;
(c) the sense strand comprises a first nucleic acid sequence having at least 95% sequence identity to SEQ ID NO: 5, and the antisense strand comprises a second nucleic acid sequence having at least 95% sequence identity to SEQ ID NO: 6;
(d) the sense strand comprises a first nucleic acid sequence having at least 95% sequence identity to SEQ ID NO: 7, and the antisense strand comprises a second nucleic acid sequence having at least 95% sequence identity to SEQ ID NO: 8;
(e) the sense strand comprises a first nucleic acid sequence having at least 95% sequence identity to SEQ ID NO: 9, and the antisense strand comprises a second nucleic acid sequence having at least 95% sequence identity to SEQ ID NO: 10;
(f) the sense strand comprises a first nucleic acid sequence having at least 95% sequence identity to SEQ ID NO: 11, and the antisense strand comprises a second nucleic acid sequence having at least 95% sequence identity to SEQ ID NO: 12;
(g) the sense strand comprises a first nucleic acid sequence having at least 95% sequence identity to SEQ ID NO: 13, and the antisense strand comprises a second nucleic acid sequence having at least 95% sequence identity to SEQ ID NO: 14;
(h) the sense strand comprises a first nucleic acid sequence having at least 95% sequence identity to SEQ ID NO: 15, and the antisense strand comprises a second nucleic acid sequence having at least 95% sequence identity to SEQ ID NO: 16;
(i) the sense strand comprises a first nucleic acid sequence having at least 95% sequence identity to SEQ ID NO: 17, and the antisense strand comprises a second nucleic acid sequence having at least 95% sequence identity to SEQ ID NO: 18;
(j) the sense strand comprises a first nucleic acid sequence having at least 95% sequence identity to SEQ ID NO: 19, and the antisense strand comprises a second nucleic acid sequence having at least 95% sequence identity to SEQ ID NO: 20;
(k) the sense strand comprises a first nucleic acid sequence having at least 95% sequence identity to SEQ ID NO: 21, and the antisense strand comprises a second nucleic acid sequence having at least 95% sequence identity to SEQ ID NO: 22;
(l) the sense strand comprises a first nucleic acid sequence having at least 95% sequence identity to SEQ ID NO: 23, and the antisense strand comprises a second nucleic acid sequence having at least 95% sequence identity to SEQ ID NO: 24;
(m) the sense strand comprises a first nucleic acid sequence having at least 95% sequence identity to SEQ ID NO: 25, and the antisense strand comprises a second nucleic acid sequence having at least 95% sequence identity to SEQ ID NO: 26;
(n) the sense strand comprises a first nucleic acid sequence having at least 95% sequence identity to SEQ ID NO: 27, and the antisense strand comprises a second nucleic acid sequence having at least 95% sequence identity to SEQ ID NO: 28; and
(o) the sense strand comprises a first nucleic acid sequence having at least 95% sequence identity to SEQ ID NO: 29, and the antisense strand comprises a second nucleic acid sequence having at least 95% sequence identity to SEQ ID NO: 30;
wherein optionally one or more nucleotides of the sense strand and the antisense strand are independently modified nucleotides, and
wherein optionally one or more internucleotide linkages of the sense strand and the antisense strand are modified internucleotide linkages.

2. The ATXN2 RNAi agent of claim 1, wherein the sense strand and the antisense strand comprise a pair of nucleic acid sequences selected from the group consisting of:
(a) the sense strand comprises a first nucleic acid sequence of SEQ ID NO: 1, and the antisense strand comprises a second nucleic acid sequence of SEQ ID NO: 2;
(b) the sense strand comprises a first nucleic acid sequence of SEQ ID NO: 3, and the antisense strand comprises a second nucleic acid sequence of SEQ ID NO: 4;
(c) the sense strand comprises a first nucleic acid sequence of SEQ ID NO: 5, and the antisense strand comprises a second nucleic acid sequence of SEQ ID NO: 6;
(d) the sense strand comprises a first nucleic acid sequence of SEQ ID NO: 7, and the antisense strand comprises a second nucleic acid sequence of SEQ ID NO: 8;
(e) the sense strand comprises a first nucleic acid sequence of SEQ ID NO: 9, and the antisense strand comprises a second nucleic acid sequence of SEQ ID NO: 10;
(f) the sense strand comprises a first nucleic acid sequence of SEQ ID NO: 11, and the antisense strand comprises a second nucleic acid sequence of SEQ ID NO: 12;
(g) the sense strand comprises a first nucleic acid sequence of SEQ ID NO: 13, and the antisense strand comprises a second nucleic acid sequence of SEQ ID NO: 14;
(h) the sense strand comprises a first nucleic acid sequence of SEQ ID NO: 15, and the antisense strand comprises a second nucleic acid sequence of SEQ ID NO: 16;
(i) the sense strand comprises a first nucleic acid sequence of SEQ ID NO: 17, and the antisense strand comprises a second nucleic acid sequence of SEQ ID NO: 18;
(j) the sense strand comprises a first nucleic acid sequence of SEQ ID NO: 19, and the antisense strand comprises a second nucleic acid sequence of SEQ ID NO: 20;
(k) the sense strand comprises a first nucleic acid sequence of SEQ ID NO: 21, and the antisense strand comprises a second nucleic acid sequence of SEQ ID NO: 22;
(l) the sense strand comprises a first nucleic acid sequence of SEQ ID NO: 23, and the antisense strand comprises a second nucleic acid sequence of SEQ ID NO: 24;
(m) the sense strand comprises a first nucleic acid sequence of SEQ ID NO: 25, and the antisense strand comprises a second nucleic acid sequence of SEQ ID NO: 26;
(n) the sense strand comprises a first nucleic acid sequence of SEQ ID NO: 27, and the antisense strand comprises a second nucleic acid sequence of SEQ ID NO: 28; and
(o) the sense strand comprises a first nucleic acid sequence of SEQ ID NO: 29, and the antisense strand comprises a second nucleic acid sequence of SEQ ID NO: 30.

3. The ATXN2 RNAi agent of claim 2, wherein one or more nucleotides of the sense strand are modified nucleotides.

4. The ATXN2 RNAi agent of claim 2, wherein each nucleotide of the sense strand is a modified nucleotide.

5. The ATXN2 RNAi agent of claim 2, wherein one or more nucleotides of the antisense strand are modified nucleotides.

6. The ATXN2 RNAi agent of claim 2, wherein each nucleotide of the antisense strand is a modified nucleotide.

7. The ATXN2 RNAi agent of claim 2, wherein the modified nucleotide is a 2′-fluoro modified nucleotide, 2′-O-methyl modified nucleotide or 2′-O-alkyl modified nucleotide.

8. The ATXN2 RNAi agent of claim 7, wherein the sense strand has four 2′-fluoro modified nucleotides at positions 7, 9, 10, and 11 from the 5′ end of the sense strand.

9. The ATXN2 RNAi agent of claim 8, wherein nucleotides at positions other than positions 7, 9, 10, and 11 of the sense strand are 2′-O-methyl modified nucleotides.

10. The ATXN2 RNAi agent of claim 2, wherein the antisense strand has four 2′-fluoro modified nucleotides at positions 2, 6, 14, and 16 from the 5′ end of the antisense strand.

11. The ATXN2 RNAi agent of claim 10, wherein nucleotides at positions other than positions 2, 6, 14 and 16 of the antisense strand are 2′-O-methyl modified nucleotides.

12. The ATXN2 RNAi agent of claim 2, wherein the sense strand and the antisense strand have one or more modified internucleotide linkages.

13. The ATXN2 RNAi agent of claim 12, wherein the modified internucleotide linkage is phosphorothioate linkage.

14. The ATXN2 RNAi agent of claim 13, wherein the sense strand has four or five phosphorothioate linkages.

15. The ATXN2 RNAi agent of claim 13, wherein the antisense strand has four or five phosphorothioate linkages.

16. The ATXN2 RNAi agent of claim 2, wherein the first nucleotide from the 5′ end of the antisense strand is a modified nucleotide that has a phosphate analog.

17. The ATXN2 RNAi agent of claim 16, wherein the phosphate analog is 5′-vinylphosphonate.

18. The ATXN2 RNAi agent of claim 2, wherein the sense strand has an abasic moiety or inverted abasic moiety.

19. An ATXN2 RNAi agent comprising a sense strand and an antisense strand,
wherein the sense strand and the antisense strand form a duplex,
wherein the sense strand and the antisense strand comprise a pair of nucleic acid sequences selected from the group consisting of:
(a) the sense strand comprises a first nucleic acid sequence of SEQ ID NO: 31, and the antisense strand comprises a second nucleic acid sequence of SEQ ID NO: 32;
(b) the sense strand comprises a first nucleic acid sequence of SEQ ID NO: 33, and the antisense strand comprises a second nucleic acid sequence of SEQ ID NO: 34;
(c) the sense strand comprises a first nucleic acid sequence of SEQ ID NO: 35, and the antisense strand comprises a second nucleic acid sequence of SEQ ID NO: 36;
(d) the sense strand comprises a first nucleic acid sequence of SEQ ID NO: 37, and the antisense strand comprises a second nucleic acid sequence of SEQ ID NO: 38;
(e) the sense strand comprises a first nucleic acid sequence of SEQ ID NO: 39, and the antisense strand comprises a second nucleic acid sequence of SEQ ID NO: 40;
(f) the sense strand comprises a first nucleic acid sequence of SEQ ID NO: 41, and the antisense strand comprises a second nucleic acid sequence of SEQ ID NO: 42;
(g) the sense strand comprises a first nucleic acid sequence of SEQ ID NO: 43, and the antisense strand comprises a second nucleic acid sequence of SEQ ID NO: 44;
(h) the sense strand comprises a first nucleic acid sequence of SEQ ID NO: 45, and the antisense strand comprises a second nucleic acid sequence of SEQ ID NO: 46;
(i) the sense strand comprises a first nucleic acid sequence of SEQ ID NO: 47, and the antisense strand comprises a second nucleic acid sequence of SEQ ID NO: 48;
(j) the sense strand comprises a first nucleic acid sequence of SEQ ID NO: 49, and the antisense strand comprises a second nucleic acid sequence of SEQ ID NO: 50;
(k) the sense strand comprises a first nucleic acid sequence of SEQ ID NO: 51, and the antisense strand comprises a second nucleic acid sequence of SEQ ID NO: 52;
(l) the sense strand comprises a first nucleic acid sequence of SEQ ID NO: 53, and the antisense strand comprises a second nucleic acid sequence of SEQ ID NO: 54;
(m) the sense strand comprises a first nucleic acid sequence of SEQ ID NO: 55, and the antisense strand comprises a second nucleic acid sequence of SEQ ID NO: 56;
(n) the sense strand comprises a first nucleic acid sequence of SEQ ID NO: 57, and the antisense strand comprises a second nucleic acid sequence of SEQ ID NO: 58;
(o) the sense strand comprises a first nucleic acid sequence of SEQ ID NO: 59, and the antisense strand comprises a second nucleic acid sequence of SEQ ID NO: 60.

20. The ATXN2 RNAi agent of claim 19, wherein the sense strand and the antisense strand have a pair of nucleic acid sequences selected from the group consisting of:
(a) the sense strand consists of a first nucleic acid sequence of SEQ ID NO: 31, and the antisense strand consists of a second nucleic acid sequence of SEQ ID NO: 32;
(b) the sense strand consists of a first nucleic acid sequence of SEQ ID NO: 33, and the antisense strand consists of a second nucleic acid sequence of SEQ ID NO: 34;
(c) the sense strand consists of a first nucleic acid sequence of SEQ ID NO: 35, and the antisense strand consists of a second nucleic acid sequence of SEQ ID NO: 36;
(d) the sense strand consists of a first nucleic acid sequence of SEQ ID NO: 37, and the antisense strand consists of a second nucleic acid sequence of SEQ ID NO: 38;
(e) the sense strand consists of a first nucleic acid sequence of SEQ ID NO: 39, and the antisense strand consists of a second nucleic acid sequence of SEQ ID NO: 40;
(f) the sense strand consists of a first nucleic acid sequence of SEQ ID NO: 41, and the antisense strand consists of a second nucleic acid sequence of SEQ ID NO: 42;
(g) the sense strand consists of a first nucleic acid sequence of SEQ ID NO: 43, and the antisense strand consists of a second nucleic acid sequence of SEQ ID NO: 44;
(h) the sense strand consists of a first nucleic acid sequence of SEQ ID NO: 45, and the antisense strand consists of a second nucleic acid sequence of SEQ ID NO: 46;
(i) the sense strand consists of a first nucleic acid sequence of SEQ ID NO: 47, and the antisense strand consists of a second nucleic acid sequence of SEQ ID NO: 48;
(j) the sense strand consists of a first nucleic acid sequence of SEQ ID NO: 49, and the antisense strand consists of a second nucleic acid sequence of SEQ ID NO: 50;
(k) the sense strand consists of a first nucleic acid sequence of SEQ ID NO: 51, and the antisense strand consists of a second nucleic acid sequence of SEQ ID NO: 52;
(l) the sense strand consists of a first nucleic acid sequence of SEQ ID NO: 53, and the antisense strand consists of a second nucleic acid sequence of SEQ ID NO: 54;
(m) the sense strand consists of a first nucleic acid sequence of SEQ ID NO: 55, and the antisense strand consists of a second nucleic acid sequence of SEQ ID NO: 56;
(n) the sense strand consists of a first nucleic acid sequence of SEQ ID NO: 57, and the antisense strand consists of a second nucleic acid sequence of SEQ ID NO: 58; and
(o) the sense strand consists of a first nucleic acid sequence of SEQ ID NO: 59, and the antisense strand consists of a second nucleic acid sequence of SEQ ID NO: 60.

21. The ATXN2 RNAi agent of claim 2, wherein the sense strand has a delivery moiety conjugated to the 3′ end of the sense strand.

22. The ATXN2 RNAi agent of claim 2, wherein the sense strand has a delivery moiety conjugated to a nucleotide of the sense strand.

23. The ATXN2 RNAi agent of claim 19, wherein the sense strand has a delivery moiety conjugated to the 3′ end or a nucleotide of the sense strand.

24. The ATXN2 RNAi agent of claim 21, wherein the delivery moiety is conjugated to the 3′ end of the sense stand via a linker selected from the group consisting of:

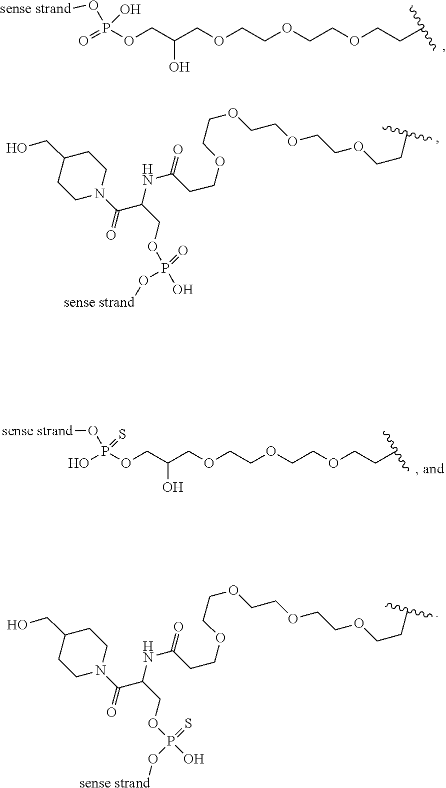

25. An ATXN2 RNAi agent of Formula (I): R-L-D,
wherein R is a double stranded RNA (dsRNA) comprising a sense stand and an antisense strand, wherein the sense strand and the antisense strand form a duplex;
wherein D is a delivery moiety; and
wherein L is a linker, or optionally absent,
wherein the sense strand and the antisense strand comprise a pair of nucleic acid sequences selected from the group consisting of:
(a) the sense strand comprises a first nucleic acid sequence of SEQ ID NO: 1, and the antisense strand comprises a second nucleic acid sequence of SEQ ID NO: 2;
(b) the sense strand comprises a first nucleic acid sequence of SEQ ID NO: 3, and the antisense strand comprises a second nucleic acid sequence of SEQ ID NO: 4;
(c) the sense strand comprises a first nucleic acid sequence of SEQ ID NO: 5, and the antisense strand comprises a second nucleic acid sequence of SEQ ID NO: 6;
(d) the sense strand comprises a first nucleic acid sequence of SEQ ID NO: 7, and the antisense strand comprises a second nucleic acid sequence of SEQ ID NO: 8;
(e) the sense strand comprises a first nucleic acid sequence of SEQ ID NO: 9, and the antisense strand comprises a second nucleic acid sequence of SEQ ID NO: 10;
(f) the sense strand comprises a first nucleic acid sequence of SEQ ID NO: 11, and the antisense strand comprises a second nucleic acid sequence of SEQ ID NO: 12;
(g) the sense strand comprises a first nucleic acid sequence of SEQ ID NO: 13, and the antisense strand comprises a second nucleic acid sequence of SEQ ID NO: 14;
(h) the sense strand comprises a first nucleic acid sequence of SEQ ID NO: 15, and the antisense strand comprises a second nucleic acid sequence of SEQ ID NO: 16;
(i) the sense strand comprises a first nucleic acid sequence of SEQ ID NO: 17, and the antisense strand comprises a second nucleic acid sequence of SEQ ID NO: 18;
(j) the sense strand comprises a first nucleic acid sequence of SEQ ID NO: 19, and the antisense strand comprises a second nucleic acid sequence of SEQ ID NO: 20;
(k) the sense strand comprises a first nucleic acid sequence of SEQ ID NO: 21, and the antisense strand comprises a second nucleic acid sequence of SEQ ID NO: 22;
(l) the sense strand comprises a first nucleic acid sequence of SEQ ID NO: 23, and the antisense strand comprises a second nucleic acid sequence of SEQ ID NO: 24;
(m) the sense strand comprises a first nucleic acid sequence of SEQ ID NO: 25, and the antisense strand comprises a second nucleic acid sequence of SEQ ID NO: 26;
(n) the sense strand comprises a first nucleic acid sequence of SEQ ID NO: 27, and the antisense strand comprises a second nucleic acid sequence of SEQ ID NO: 28; and
(o) the sense strand comprises a first nucleic acid sequence of SEQ ID NO: 29, and the antisense strand comprises a second nucleic acid sequence of SEQ ID NO: 30, wherein optionally one or more nucleotides of the sense strand and the antisense strand are independently modified nucleotides, and
wherein optionally one or more internucleotide linkages of the sense strand and the antisense strand are modified internucleotide linkages.

26. An ATXN2 RNAi agent of Formula (I): R-L-D,
wherein R is a double stranded RNA (dsRNA) comprising a sense stand and an antisense strand, wherein the sense strand and the antisense strand form a duplex;
wherein D is a delivery means for delivering the dsRNA into a cell; and
wherein L is a linking means for linking the dsRNA to the delivery means, or optionally absent,
wherein the sense strand and the antisense strand comprise a pair of nucleic acid sequences selected from the group consisting of:
(a) the sense strand comprises a first nucleic acid sequence of SEQ ID NO: 1, and the antisense strand comprises a second nucleic acid sequence of SEQ ID NO: 2;
(b) the sense strand comprises a first nucleic acid sequence of SEQ ID NO: 3, and the antisense strand comprises a second nucleic acid sequence of SEQ ID NO: 4;
(c) the sense strand comprises a first nucleic acid sequence of SEQ ID NO: 5, and the antisense strand comprises a second nucleic acid sequence of SEQ ID NO: 6;
(d) the sense strand comprises a first nucleic acid sequence of SEQ ID NO: 7, and the antisense strand comprises a second nucleic acid sequence of SEQ ID NO: 8;
(e) the sense strand comprises a first nucleic acid sequence of SEQ ID NO: 9, and the antisense strand comprises a second nucleic acid sequence of SEQ ID NO: 10;
(f) the sense strand comprises a first nucleic acid sequence of SEQ ID NO: 11, and the antisense strand comprises a second nucleic acid sequence of SEQ ID NO: 12;
(g) the sense strand comprises a first nucleic acid sequence of SEQ ID NO: 13, and the antisense strand comprises a second nucleic acid sequence of SEQ ID NO: 14;
(h) the sense strand comprises a first nucleic acid sequence of SEQ ID NO: 15, and the antisense strand comprises a second nucleic acid sequence of SEQ ID NO: 16;
(i) the sense strand comprises a first nucleic acid sequence of SEQ ID NO: 17, and the antisense strand comprises a second nucleic acid sequence of SEQ ID NO: 18;
(j) the sense strand comprises a first nucleic acid sequence of SEQ ID NO: 19, and the antisense strand comprises a second nucleic acid sequence of SEQ ID NO: 20;
(k) the sense strand comprises a first nucleic acid sequence of SEQ ID NO: 21, and the antisense strand comprises a second nucleic acid sequence of SEQ ID NO: 22;
(l) the sense strand comprises a first nucleic acid sequence of SEQ ID NO: 23, and the antisense strand comprises a second nucleic acid sequence of SEQ ID NO: 24;
(m) the sense strand comprises a first nucleic acid sequence of SEQ ID NO: 25, and the antisense strand comprises a second nucleic acid sequence of SEQ ID NO: 26;
(n) the sense strand comprises a first nucleic acid sequence of SEQ ID NO: 27, and the antisense strand comprises a second nucleic acid sequence of SEQ ID NO: 28; and
(o) the sense strand comprises a first nucleic acid sequence of SEQ ID NO: 29, and the antisense strand comprises a second nucleic acid sequence of SEQ ID NO: 30,
wherein optionally one or more nucleotides of the sense strand and the antisense strand are independently modified nucleotides, and
wherein optionally one or more internucleotide linkages of the sense strand and the antisense strand are modified internucleotide linkages.

27. The ATXN2 RNAi agent of claim 25, wherein the sense strand and the antisense strand comprise a pair of nucleic acid sequences selected from the group consisting of:
(a) the sense strand comprises a first nucleic acid sequence of SEQ ID NO: 31, and the antisense strand comprises a second nucleic acid sequence of SEQ ID NO: 32;
(b) the sense strand comprises a first nucleic acid sequence of SEQ ID NO: 33, and the antisense strand comprises a second nucleic acid sequence of SEQ ID NO: 34;
(c) the sense strand comprises a first nucleic acid sequence of SEQ ID NO: 35, and the antisense strand comprises a second nucleic acid sequence of SEQ ID NO: 36;
(d) the sense strand comprises a first nucleic acid sequence of SEQ ID NO: 37, and the antisense strand comprises a second nucleic acid sequence of SEQ ID NO: 38;
(e) the sense strand comprises a first nucleic acid sequence of SEQ ID NO: 39, and the antisense strand comprises a second nucleic acid sequence of SEQ ID NO: 40;
(f) the sense strand comprises a first nucleic acid sequence of SEQ ID NO: 41, and the antisense strand comprises a second nucleic acid sequence of SEQ ID NO: 42;
(g) the sense strand comprises a first nucleic acid sequence of SEQ ID NO: 43, and the antisense strand comprises a second nucleic acid sequence of SEQ ID NO: 44;
(h) the sense strand comprises a first nucleic acid sequence of SEQ ID NO: 45, and the antisense strand comprises a second nucleic acid sequence of SEQ ID NO: 46;
(i) the sense strand comprises a first nucleic acid sequence of SEQ ID NO: 47, and the antisense strand comprises a second nucleic acid sequence of SEQ ID NO: 48;
(j) the sense strand comprises a first nucleic acid sequence of SEQ ID NO: 49, and the antisense strand comprises a second nucleic acid sequence of SEQ ID NO: 50;
(k) the sense strand comprises a first nucleic acid sequence of SEQ ID NO: 51, and the antisense strand comprises a second nucleic acid sequence of SEQ ID NO: 52;
(l) the sense strand comprises a first nucleic acid sequence of SEQ ID NO: 53, and the antisense strand comprises a second nucleic acid sequence of SEQ ID NO: 54;
(m) the sense strand comprises a first nucleic acid sequence of SEQ ID NO: 55, and the antisense strand comprises a second nucleic acid sequence of SEQ ID NO: 56;
(n) the sense strand comprises a first nucleic acid sequence of SEQ ID NO: 57, and the antisense strand comprises a second nucleic acid sequence of SEQ ID NO: 58; and
(o) the sense strand comprises a first nucleic acid sequence of SEQ ID NO: 59, and the antisense strand comprises a second nucleic acid sequence of SEQ ID NO: 60.

28. The ATXN2 RNAi agent of claim 25,
wherein the sense strand and the antisense strand have a pair of nucleic acid sequences selected from the group consisting of:
(a) the sense strand consists of a first nucleic acid sequence of SEQ ID NO: 31, and the antisense strand consists of a second nucleic acid sequence of SEQ ID NO: 32;
(b) the sense strand consists of a first nucleic acid sequence of SEQ ID NO: 33, and the antisense strand consists of a second nucleic acid sequence of SEQ ID NO: 34;
(c) the sense strand consists of a first nucleic acid sequence of SEQ ID NO: 35, and the antisense strand consists of a second nucleic acid sequence of SEQ ID NO: 36;
(d) the sense strand consists of a first nucleic acid sequence of SEQ ID NO: 37, and the antisense strand consists of a second nucleic acid sequence of SEQ ID NO: 38;
(e) the sense strand consists of a first nucleic acid sequence of SEQ ID NO: 39, and the antisense strand consists of a second nucleic acid sequence of SEQ ID NO: 40;
(f) the sense strand consists of a first nucleic acid sequence of SEQ ID NO: 41, and the antisense strand consists of a second nucleic acid sequence of SEQ ID NO: 42;
(g) the sense strand consists of a first nucleic acid sequence of SEQ ID NO: 43, and the antisense strand consists of a second nucleic acid sequence of SEQ ID NO: 44;
(h) the sense strand consists of a first nucleic acid sequence of SEQ ID NO: 45, and the antisense strand consists of a second nucleic acid sequence of SEQ ID NO: 46;
(i) the sense strand consists of a first nucleic acid sequence of SEQ ID NO: 47, and the antisense strand consists of a second nucleic acid sequence of SEQ ID NO: 48;
(j) the sense strand consists of a first nucleic acid sequence of SEQ ID NO: 49, and the antisense strand consists of a second nucleic acid sequence of SEQ ID NO: 50;
(k) the sense strand consists of a first nucleic acid sequence of SEQ ID NO: 51, and the antisense strand consists of a second nucleic acid sequence of SEQ ID NO: 52;
(l) the sense strand consists of a first nucleic acid sequence of SEQ ID NO: 53, and the antisense strand consists of a second nucleic acid sequence of SEQ ID NO: 54;
(m) the sense strand consists of a first nucleic acid sequence of SEQ ID NO: 55, and the antisense strand consists of a second nucleic acid sequence of SEQ ID NO: 56;
(n) the sense strand consists of a first nucleic acid sequence of SEQ ID NO: 57, and the antisense strand consists of a second nucleic acid sequence of SEQ ID NO: 58; and
(o) the sense strand consists of a first nucleic acid sequence of SEQ ID NO: 59, and the antisense strand consists of a second nucleic acid sequence of SEQ ID NO: 60.

29. The ATXN2 RNAi agent of claim 25, wherein the delivery moiety is conjugated to the 3′ end of the sense strand.

30. The ATXN2 RNAi agent of claim 25, wherein the delivery moiety is conjugated to a nucleotide of the sense strand.

31. A pharmaceutical composition comprising the ATXN2 RNAi agent of claim 2 and a pharmaceutically acceptable carrier.

32. A pharmaceutical composition comprising a means for reducing ATXN2 expression in a cell and a pharmaceutically acceptable carrier.

33. A method of reducing ATXN2 expression in a patient in need thereof, the method comprising administering to the patient an effective amount of the ATXN2 RNAi agent of claim 2.

34. A method of treating an ATXN2-associated neurological disease in a patient in need thereof, the method comprising administering to the patient an effective amount of the ATXN2 RNAi agent of claim 2.

35. The method of claim 34, wherein the ATXN2-associated neurological disease is selected from spinocerebellar ataxia type 2 (SCA2), amyotrophic lateral sclerosis (ALS), primary lateral sclerosis (PLS), Parkinson's disease, Alzheimer's disease, frontotemporal lobar degeneration (FTLD), progressive muscular atrophy (PMA), multiple system proteinopathy, Perry disease, or TDP-43 proteinopathy.

36. (canceled)

37. The method of claim 34, wherein the ATXN2 RNAi agent is administered to the patient intrathecally.

38. A method of reducing ATXN2 expression in a cell, the method comprising:
introducing the ATXN2 RNAi agent of claim 2 into the cell; and
incubating the cell for a time sufficient for degradation of ATXN2 mRNA, thereby reducing ATXN2 expression in the cell.

39.-46. (canceled)

47. A method of treating an ATXN2-associated neurological disease in a patient in need thereof, the method comprising administering to the patient an effective amount of the ATXN2 RNAi agent of claim 19.

48. The method of claim 47, wherein the ATXN2-associated neurological disease is selected from spinocerebellar ataxia type 2 (SCA2), amyotrophic lateral sclerosis (ALS), primary lateral sclerosis (PLS), Parkinson's disease, Alzheimer's disease, frontotemporal lobar degeneration (FTLD), progressive muscular atrophy (PMA), multiple system proteinopathy, Perry disease, or TDP-43 proteinopathy.

49. A method of treating an ATXN2-associated neurological disease in a patient in need thereof, the method comprising administering to the patient an effective amount of the ATXN2 RNAi agent of claim 25.

50. The method of claim 49, wherein the ATXN2-associated neurological disease is selected from spinocerebellar ataxia type 2 (SCA2), amyotrophic lateral sclerosis (ALS), primary lateral sclerosis (PLS), Parkinson's disease, Alzheimer's disease, frontotemporal lobar degeneration (FTLD), progressive muscular atrophy (PMA), multiple system proteinopathy, Perry disease, or TDP-43 proteinopathy.
